(19) United States
(12) Patent Application Publication
(10) Pub. No.: US 2016/0166280 A1
(43) Pub. Date: Jun. 16, 2016

(54) PUNCTURE APPARATUS AND PUNCTURING METHOD

(71) Applicant: TERUMO KABUSHIKI KAISHA, Tokyo (JP)

(72) Inventors: Yuusuke TAKAHASHI, Hadano-shi (JP); Masakatsu KAWAURA, Sunnyvale, CA (US)

(73) Assignee: TERUMO KABUSHIKI KAISHA, Tokyo (JP)

(21) Appl. No.: 15/050,981

(22) Filed: Feb. 23, 2016

Related U.S. Application Data
(63) Continuation of application No. PCT/JP2014/074259, filed on Sep. 12, 2014.

(30) Foreign Application Priority Data
Sep. 21, 2013 (JP) 2013-196274

(51) Int. Cl. A61B 17/34; A61F 2/00

(52) U.S. Cl. CPC A61B 17/3468; A61F 2/0045; A61F 2002/0072; A61B 2017/3492

(57) ABSTRACT

A puncture apparatus is disclosed, which includes a first puncture needle which is curved, the first puncture needle provided in a rotationally movable manner; and a second puncture needle which is curved, the second puncture needle provided in a rotationally movable manner. When the first puncture needle and the second puncture needle are both rotationally moved toward their distal sides, a distal portion of the first puncture needle and a distal portion of the second puncture needle engage each other.

### CROSS-REFERENCES TO RELATED APPLICATIONS

This application is a continuation of International Application No. PCT/JP2014/074259 filed on Sep. 12, 2014, and claims priority to Japanese Application No. 2013-196274 filed on Sep. 21, 2013, the entire content of both of which is incorporated herein by reference.

### TECHNICAL FIELD

The present disclosure generally relates to a puncture apparatus and a puncturing method.

### BACKGROUND DISCUSSION

In a patient suffering from urinary incontinence, particularly stress urinary incontinence, urine leakage can occur due to an abdominal pressure exerted during a normal exercise or by laughing, coughing, sneezing or the like. This can be attributable, for example, to loosening of the pelvic floor muscle, which is a muscle for supporting the urethra, caused by childbirth or the like.

For treatment of urinary incontinence, surgical treatment can be effective, in which there is used, for example, a belt-shaped living body tissue-supporting indwelling article called “sling.” The sling is placed indwelling in the body and the urethra is supported by the sling (see, for example, Japanese Patent Laid-Open No. 2010-99499). In order to put a sling indwelling inside the body, an operator would incise the vagina with a surgical knife, dissect the living body tissue between the urethra and vagina, and make the dissected region and the outside communicate with each other through obturator foramens by use of a puncture needle or the like. Then, in this state, the sling is placed indwelling in the body.

If the vagina is incised once, however, a situation may occur that the sling is exposed to the inside of the vagina from a wound caused by the incision of the vagina, and complications may be caused by an infection from the wound. Further, since the vagina is incised, the burden on the patient can be relatively heavy. Further, the urethra or the like may be damaged in the course of the procedure by the operator. In addition, the fingertip of the operator himself/herself may be damaged or injured.

### SUMMARY

Disclosed herein are a puncture apparatus and a puncturing method by which a living body tissue-supporting indwelling article can be relatively easily embedded into a living body while help ensure a reduced burden on the patient and an enhanced safety for the patient.

A puncture apparatus is disclosed, which includes a first puncture needle which is curved, the first puncture needle provided in a rotationally movable manner; and a second puncture needle which is curved, the second puncture needle provided in a rotationally movable manner. In the puncture apparatus, when the first puncture needle and the second puncture needle are both rotationally moved toward their distal sides, a distal portion of the first puncture needle and a distal portion of the second puncture needle engage each other.

In accordance with an exemplary embodiment, the puncture apparatus further includes a urethra-insertion portion having an elongated shape, the urethra-insertion portion configured to be inserted into a urethra; and a restriction unit adapted to restrict positional relationships between the first puncture needle and the second puncture needle and the urethra-insertion portion so that when the first puncture needle and the second puncture needle are rotationally moved toward their distal sides, a distal end of at least one of the first puncture needle and the second puncture needle passes on a farther side from a rotational center of the first puncture needle and the second puncture needle than the urethral-insertion portion.

In accordance with an exemplary embodiment, the puncture apparatus further includes a vaginal-insertion portion having an elongated shape, the vaginal-insertion portion configured to be inserted into a vagina. In addition, in the puncture apparatus, preferably, the restriction unit restricts positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion.

In accordance with an exemplary embodiment, the puncture apparatus further includes a first shaft portion connected to the first puncture needle, the first shaft portion constituting a rotational shaft for rotary movement of the first puncture needle; and a second shaft portion connected to the second puncture needle, the second shaft portion constituting a rotational shaft for rotary movement of the second puncture needle. Besides, in the puncture apparatus, preferably, the restriction unit has a support portion supporting the first shaft portion and the second shaft portion in a rotationally movable manner, the support portion supporting the urethral-insertion portion.

In accordance with an exemplary embodiment, the puncture apparatus further includes a vaginal-insertion portion having an elongated shape, the vaginal-insertion portion configured to be inserted into a vagina; a first shaft portion connected to the first puncture needle, the first shaft portion constituting a rotational shaft for rotary movement of the first puncture needle; and a second shaft portion connected to the second puncture needle, the second shaft portion constituting a rotational shaft for rotary movement of the second puncture needle. In addition, in the puncture apparatus, preferably, the restriction unit restricts positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion, and the restriction unit has a support portion supporting the first shaft portion and the second shaft portion in a rotationally movable manner, the support portion supporting the urethral-insertion portion and the vaginal-insertion portion.

In accordance with an exemplary embodiment, a rotational axis of each of the first puncture needle and the second puncture needle is inclined against an axis of the urethral-insertion portion so that a separated distance between the rotational axis and the axis of the urethral-insertion portion increases or decreases along a distal direction.

A puncturing method is disclosed, which includes rotationally moving a first puncture needle toward its distal side to cause the first puncture needle to puncture a living body from a first part of the living body; rotationally moving a second puncture needle toward its distal side to cause the second puncture needle to puncture the living body from a second part different from the first part of the living body; and causing a distal portion of the first puncture needle and a distal portion of the second puncture needle to engage each other in the living body.

In accordance with an exemplary embodiment, a through-hole can be formed in a living body by use of the first puncture needle and the second puncture needle. Therefore, the first puncture needle and the second puncture needle can be made shorter in length, and deformation (flexure, torsion or the like) of the first puncture needle and the second puncture needle at the time of puncturing can be restrained, which can help ensure accurate puncturing by use of the puncture needles. Further, the living body tissue-supporting indwelling article (sling) can be easily embedded into a living body while ensuring a reduced burden on the patient and an enhanced safety for the patient.

### BRIEF DESCRIPTION OF THE DRAWINGS

FIG. 1 is a perspective view illustrating one embodiment of an implant;

FIG. 2 is a perspective view illustrating a puncture apparatus according to a first embodiment of the present disclosure;

FIG. 3 is a lateral view of the puncture apparatus of FIG. 2;

FIG. 4 is a plan view illustrating puncture members possessed by the puncture apparatus of FIG. 2;

FIGS. 5A and 5B are plan views for explaining a method of connecting two split pieces put over the puncture member depicted in FIG. 4;

FIG. 6 is a sectional view taken along line VI-VI of FIG. 5A;

FIGS. 7A and 7B are views illustrating a sheath possessed by the puncture apparatus of FIG. 2, wherein FIG. 7A is a perspective view, and FIG. 7B is a sectional view taken along line VIIB-VIIB of FIG. 7A;

FIG. 8 is a plan view illustrating a fixation portion of a frame provided in the puncture apparatus of FIG. 2;

FIG. 9 is a sectional view illustrating a rotational operation unit provided on the frame provided in the puncture apparatus of FIG. 2;

FIG. 10 is a lateral view of an insertion device possessed by the puncture apparatus of FIG. 2;

FIGS. 11A and 11B are views illustrating positional relationship between puncture members and obturator foramens (pelvis), wherein FIG. 11A is a lateral view, and FIG. 11B is a front view;

FIG. 12 is a partial enlarged view of a vaginal-insertion device possessed by the insertion device of FIG. 10;

FIG. 13A is a sectional view illustrating one example of the shape of a vaginal wall, and FIG. 13B is a sectional view illustrating a condition where a vaginal-insertion portion is inserted in a vagina depicted in FIG. 13A;

FIGS. 14A and 14B are views for explaining an operating procedure of the puncture apparatus of FIG. 2;

FIGS. 15A and 15B are views for explaining an operating procedure of the puncture apparatus of FIG. 2;

FIG. 16 is a lateral view illustrating the relationship between the puncture apparatus and the pelvis at the time of the state depicted in FIG. 15A;

FIGS. 17A and 17B are views for explaining an operating procedure of the puncture apparatus of FIG. 2;

FIG. 18 is a lateral view illustrating the relationship between the puncture apparatus and the pelvis at the time of the state as depicted in FIG. 17A;

FIG. 19 is a sectional view illustrating the posture of the sheath relative to a urethra at the time of the state as depicted in FIG. 17B;

FIGS. 20A and 20B are views for explaining an operating procedure of the puncture apparatus of FIG. 2;

FIG. 21 is a sectional view illustrating first and second puncture needles possessed by a puncture apparatus according to a second embodiment of the present disclosure, depicting a state where the first and second puncture needles are in engagement with each other;

FIG. 22 is a perspective view illustrating a puncture apparatus according to a third embodiment of the present disclosure;

FIG. 23 is a sectional view illustrating first and second puncture needles possessed by the puncture apparatus of FIG. 22, depicting a state where the first and second puncture needles are in engagement with each other;

FIGS. 24A and 24B are views for explaining an operating procedure of the puncture apparatus of FIG. 22;

FIG. 25 is a view for explaining an operating procedure of the puncture apparatus of FIG. 22;

FIG. 26 is a perspective view illustrating a puncture apparatus according to a fourth embodiment of the present disclosure;

FIG. 27A is a perspective view illustrating a distal portion of a first puncture needle, and FIG. 27B is a perspective view illustrating a distal portion of a second puncture needle;

FIGS. 28A and 28B are sectional views for explaining transfer of an implant from a first puncture needle to a second puncture needle;

FIGS. 29A and 29B are views for explaining an operating procedure of the puncture apparatus of FIG. 26;

FIG. 30A is a sectional view illustrating first and second puncture needles possessed by a puncture apparatus according to a fifth embodiment of the present disclosure, and FIGS. 30B and 30C are sectional views for explaining transfer of an implant from the first puncture needle to the second puncture needle;

FIG. 31 is a perspective view illustrating a puncture apparatus according to a sixth embodiment of the present disclosure;

FIG. 32 is a perspective view illustrating puncture members possessed by the puncture apparatus of FIG. 31;

FIG. 33 is a top plan view of the puncture members depicted in FIG. 32;

FIGS. 34A and 34B are plan views for explaining rotary movement of the puncture members depicted in FIG. 32, wherein FIG. 34A depicts an initial state, and FIG. 34B depicts a puncturing state;

FIG. 35 is a top plan view illustrating puncture members possessed by a puncture apparatus according to a seventh embodiment of the present disclosure;

FIG. 36 is a perspective view illustrating puncture members possessed by a puncture apparatus according to an eighth embodiment of the present disclosure;

FIG. 37 is a sectional view illustrating a rotational operation unit possessed by the puncture apparatus according to the eighth embodiment of the present disclosure;

FIGS. 38A and 38B are plan views illustrating puncture members possessed by a puncture apparatus according to a ninth embodiment of the present disclosure, wherein FIG. 38A depicts an initial state, and FIG. 38B depicts a puncturing state;

FIG. 39 is a top plan view of the puncture members depicted in FIGS. 38A and 38B;

FIGS. 40A and 40B are plan views illustrating puncture members possessed by a puncture apparatus according to a tenth embodiment of the present disclosure, wherein FIG. 40A depicts an initial state, and FIG. 40B depicts a puncturing state;

FIGS. 41A and 41B are plan views illustrating a modification of the puncture member depicted in FIG. 40;

FIGS. 42A and 42B are plan views illustrating puncture members possessed by a puncture apparatus according to an eleventh embodiment of the present disclosure, wherein FIG. 42A depicts an initial state, and FIG. 42B depicts a puncturing state;

FIG. 43 is a plan view illustrating puncture members possessed by a puncture apparatus according to a twelfth embodiment of the present disclosure;

FIGS. 44A and 44B are views for explaining an operating procedure of the puncture apparatus possessed by the puncture member depicted in FIG. 43;

FIGS. 45A to 45C are views illustrating an initial state of a puncture apparatus according to a thirteenth embodiment of the present disclosure, wherein FIG. 45A is a front view, FIG. 45B is a top plan view, and FIG. 45C is a bottom view;

FIGS. 46A and 46B are views for explaining an operating procedure of the puncture apparatus of FIGS. 45A to 45C;

FIG. 47 is a perspective view illustrating a puncture apparatus according to a fourteenth embodiment of the present disclosure;

FIG. 48 is a plan view illustrating a configuration of a rotational operation unit possessed by the puncture apparatus of FIG. 47;

FIGS. 49A and 49B are views illustrating an initial state of a puncture apparatus according to a fifteenth embodiment of the present disclosure, wherein FIG. 49A is a sectional view as viewed from a front side, and FIG. 49B is a back view;

FIG. 50 is a perspective view illustrating a puncture apparatus according to a sixteenth embodiment of the present disclosure;

FIGS. 51A and 51B are views illustrating a configuration of a rotational operation unit possessed by the puncture apparatus of FIG. 50, wherein FIG. 51A is a top plan view, and FIG. 51B is a front view; and

FIGS. 52A and 52B are views illustrating an initial state of a puncture apparatus according to a seventeenth embodiment of the present disclosure, wherein FIG. 52A is a sectional view as viewed from a front side, and FIG. 52B is a back view.

### DETAILED DESCRIPTION

A puncture apparatus and a puncturing method according to the present disclosure will now be described in detail below, referring to preferred embodiments thereof illustrated in the attached drawings.

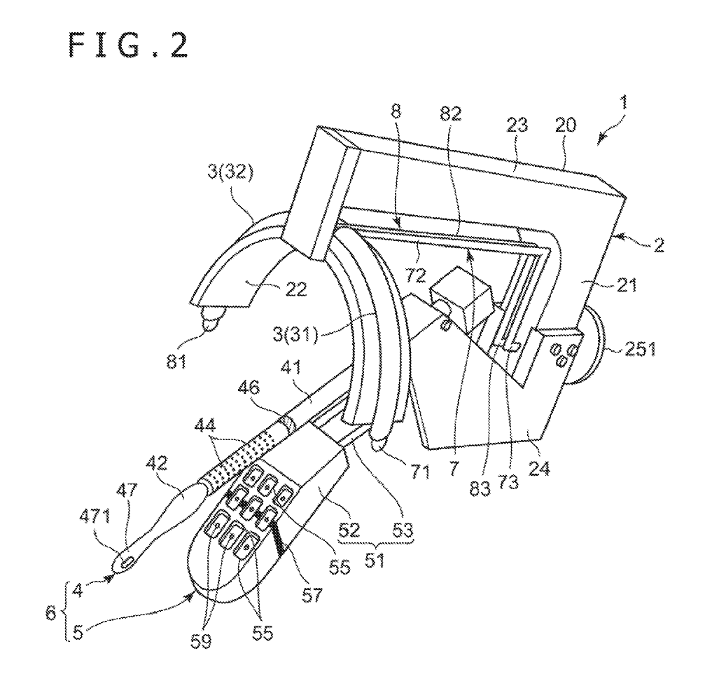

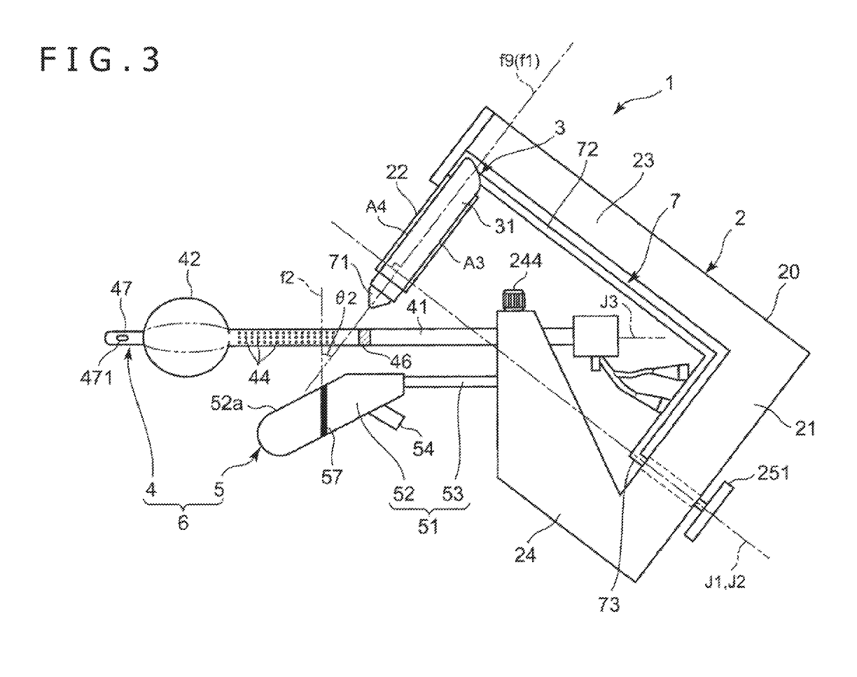

Note that in the following, for convenience of explanation, the left side in FIG. 3 will be referred to also as “distal (end),” the right side in FIG. 3 will be referred to also as “proximal (end),” the upper side in FIG. 3 will be referred to also as “upper (side),” and the lower side in FIG. 3 will be referred to also as “lower (side).” In addition, FIGS. 2 to 4 illustrate a state where a puncture apparatus (insertion device) is not yet used, and this state will hereinafter be referred to as “initial state” for convenience of explanation. In addition, a state where a puncture apparatus (insertion device) depicted in FIG. 3 is mounted onto a patient will be referred to as “mounted state” for convenience of explanation.

In accordance with an exemplary embodiment, one example of an implant (living body tissue-supporting indwelling article) 9 to be embedded in a living body by a puncture apparatus will be described.

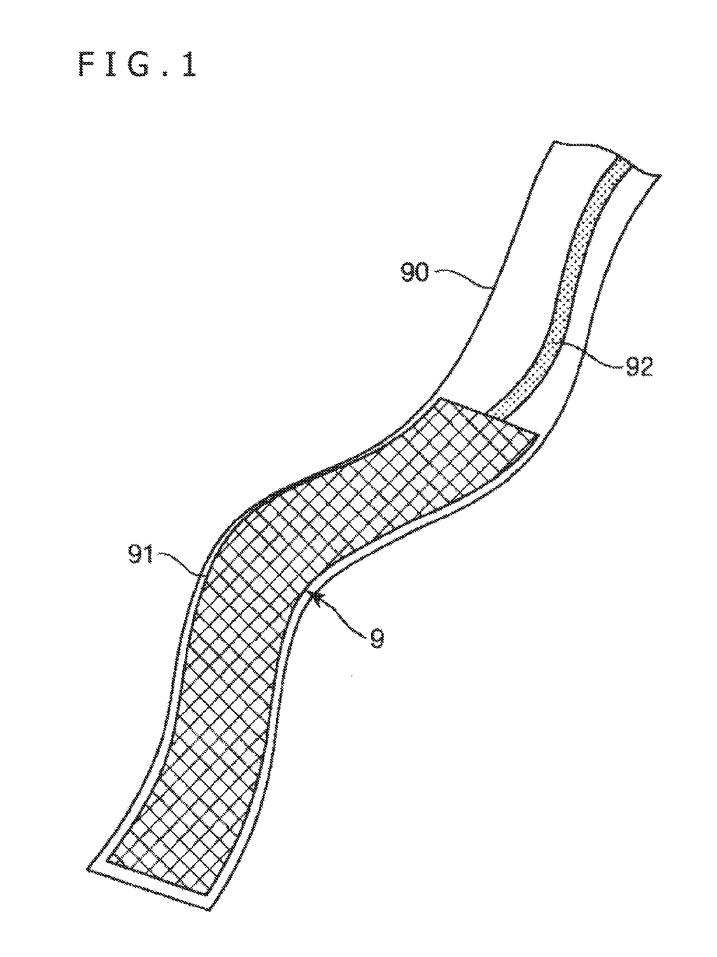

An implant 9 illustrated in FIG. 1 is a device which can be embedded in a living body for treatment of female urinary incontinence. More specifically, the implant 9 is a device for supporting a urethra, for example, a device which, when the urethra is going to move toward the vaginal-wall side, supports the urethra so as to restrict such a movement, in the manner of pulling the urethra in a direction for spacing away from the vaginal wall. As such, an implant 9, for example, an elongated body having flexibility can be used.

As depicted in FIG. 1, the implant 9 can include an implant main body 91, and a belt (connection portion) 92 connected to one end of the implant main body 91. Note that, for example, a guide wire, a cord, a string, or the like may be used in place of the belt 92. The implant main body 91 can be net-like in form, and can be belt-like in overall shape. The implant main body 91 may be composed, for example, of a net-like knitted body formed by intersecting linear (filamentous) members, or a net-like braiding. Examples of the linear (filamentous member) include those, which are circular in cross-sectional shape, and those, which are flattened in cross-sectional shape, namely, tape-like members.

The implant 9 configured in this way is sterilized and accommodated in a wrapping material 90 in its unused state. By this, the implant 9 can be prevented against contamination.

The materials constituting the implant main body 91, the belt 92, and the wrapping material 90 are not particularly limited. Examples of usable materials include various biocompatible resin materials such as polypropylene, polyester, nylon, etc. and their fibers.

While the implant 9 has been described above, the implant 9 is not limited to the net-like one, so long as the implant 9 can exhibit the equivalent or similar effect to the above-mentioned.

A puncture apparatus 1 illustrated in FIG. 2 can be an apparatus for use in embedding the aforementioned implant 9 into a living body.

As depicted in FIG. 2, the puncture apparatus 1 can include a frame (restriction unit) 2, an insertion device 6, two puncture members 7 and 8, and a sheath (medical tube) 3. The insertion device 6 and the puncture members 7 and 8 are supported by the frame 2. The sheath 3 is supported by the puncture members 7 and 8. In addition, the insertion device 6 can include a urethral-insertion device 4 and a vaginal-insertion device 5.

The puncture members 7 and 8 are members for puncturing a living body. These puncture members 7 and 8 are provided substantially in left-right symmetry about the center of the frame 2.

As illustrated in FIGS. 2 to 5B, the puncture member (first puncture member) 7 can include a puncture needle (first puncture needle) 71, a shaft portion (first shaft portion) 73, and an interlock portion 72 interlocking the puncture needle 71 and the shaft portion 73. The puncture needle 71 is a part adapted to puncture a living body, is solid, is curved in a substantially arcuate shape, and has a cutting edge surface 711 at the distal end (needle tip) of the puncture needle 71. In addition, the shaft portion 73 extends on an axis J1 which intersects the center of the puncture needle 71 and which is orthogonal to a plane f1 containing an extension axis of the puncture needle 71. In accordance with an exemplary embodiment, the interlock portion 72 interlocks a proximal portion of the puncture needle 71 and a distal portion of the shaft portion 73, and is in a substantially L-shaped form while being bent substantially at a right angle at an intermediate portion of the interlock portion 72. Note that the puncture needle 71, the interlock portion 72, and the shaft portion 73 may be formed integrally, or at least a part thereof may be formed as a separate body from other part.

Similarly, the puncture member (second puncture member) 8 can include a puncture needle (second puncture needle) 81, a shaft portion (second shaft portion) 83, and an interlock portion 82 interlocking the puncture needle 81 and the shaft portion 83. The puncture needle 81 is a part adapted to puncture a living body, is solid, is curved in a substantially arcuate shape, and has a cutting edge surface 811 at a distal end of the puncture needle 81. The puncture needle 81 has a radius of curvature and a center angle, which are substantially equal to those of the puncture needle 71. In addition, an extension axis of the puncture needle 81 is located in the same plane f1 as that in which the extension axis of the puncture needle 71 is located. In accordance with an exemplary embodiment, the shaft portion 83 extends on an axis J2 which intersects the center of the puncture needle 81 and which is orthogonal to the plane f1. The interlock portion 82 interlocks a proximal portion of the puncture needle 81 and a distal portion of the shaft portion 83, and is in a substantially L-shaped form while being bent substantially at a right angle at an intermediate portion of the interlock portion 82. In accordance with an exemplary embodiment, the puncture needle 81, the interlock portion 82, and the shaft portion 83 may be formed integrally, or at least a part thereof may be formed as a separate body from other part.

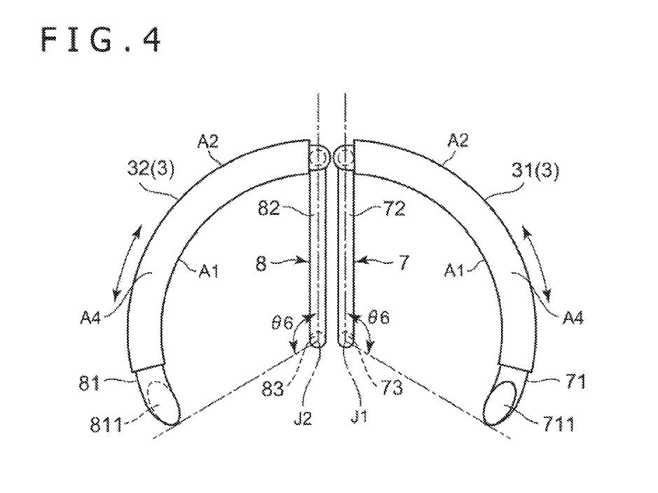

As depicted in FIGS. 2 and 4, the puncture needles 71 and 81 are disposed so that their proximal ends abut on each other in a central area, with their distal ends oriented to opposite sides. Therefore, the puncture needle 71 is made to puncture a living body by being rotationally moved clockwise (in a first direction) in FIG. 4 from an initial state, whereas the puncture needle 81 is made to puncture a living body by being rotationally moved counterclockwise (in a second direction) in FIG. 4 from an initial state. Then, finally, the distal ends of the puncture needles 71 and 81 attach each other, and the cutting edge surfaces 711 and 811 face and overlap each other. In contrast, the puncture needle 71 is drawn out of the living body by being rotationally moved counterclockwise in FIG. 4 from a puncturing state, whereas the puncture needle 81 is drawn out of the living body by being rotationally moved clockwise in FIG. 4 from a puncturing state. Particularly, because the puncture needles 71 and 81 are both curved in the arcuate shape, the puncturing of the living body and the drawing-out from the living body as above-mentioned can be performed with enhanced smoothness.

The axes J1 and J2 are provided substantially in parallel to and close to each other. Owing to this arrangement, the distal ends of the puncture members 7 and 8 can be moved substantially along a circumference of the same circle. Accordingly, puncturing by the puncture needles 71 and 81 can be performed with enhanced smoothness. In addition, puncture routes of the puncture needles 71 and 81 can be set shorter. In accordance with an exemplary embodiment, the puncture needles 71 and 81 are both moved in the plane f1, which also contributes to smoother puncturing by the puncture needles 71 and 81. Further, the cutting edge surfaces 711 and 811 can overlap each other with enhanced ease.

Note that the separated distance between the axes J1 and J2 is preferably as short as possible in such a range as not to hinder the rotary movements of the puncture members 7 and 8. Specifically, the separated distance can be preferably, for example, not more than about 10 mm, more preferably, not more than about 5 mm.

Center angles θ6 of the puncture needles 71 and 81 are not particularly limited but may be appropriately set according to various conditions. The center angles θ6 are set that the distal end of the puncture needle 71 can enter a living body from a patient's inguinal region on one side and pass through an obturator foramen on one side to reach a region between a urethra and a vagina, whereas the distal end of the puncture needle 81 can enter the living body from the patient's inguinal region on the other side and pass through an obturator foramen on the other side to reach the region between the urethra and the vagina. Specifically, the center angles θ6 of the puncture needles 71 and 81 are preferably, for example, about 80 degrees to 180 degrees, more preferably about 100 degrees to 160 degrees. By such a setting, aforesaid effects can be produced, and the puncture needles 71 and 81 can be prevented from becoming excessively long.

As will be described later, a split piece 31 is put over the puncture needle 71 in a covering manner, and a split piece 32 is put over the puncture needle 81 in a covering manner. Therefore, the shapes of the puncture needles 71 and 81 are designed in conformity with the shapes of internal spaces of the split pieces 31 and 32. More specifically, cross-sectional shapes of the puncture needles 71 and 81 are designed to be substantially equal to or slightly smaller than cross-sectional shapes of the internal spaces of the split pieces 31 and 32. This design can help ensure that the puncture needles 71 and 81 function as stylets for reinforcing the split pieces 31 and 32, whereby the split pieces 31 and 32 can be restrained from deformation. Note that the shapes of the puncture needles 71 and 81 may not necessarily be designed in conformity with the shapes of the internal spaces of the split pieces 31 and 32, so long as the puncture needles 71 and 81 can be inserted into the split pieces 31 and 32. For example, the puncture needles 71 and 81 may be circular, elliptic, tetragonal or the like in cross-sectional shape.

The materials constituting the puncture members 7 and 8 configured as above are not specifically restricted. Examples of usable materials can include various metallic materials such as stainless steel, aluminum or aluminum alloys, titanium or titanium alloys, etc.

A sheath 3 is a member which is tube-like in form and is used for placing an implant 9 indwelling in a living body by utilizing an internal space of the sheath 3.

In an initial state depicted in FIG. 4, the sheath 3 is separated into two tube-shaped split pieces 31 and 32 which are connectable. The split piece (first medical tube) 31 is coveringly put over the puncture needle 71 in a withdrawable manner, and the split piece (second medical tube) 32 is coveringly put over the puncture needle 81 in a withdrawable manner. In addition, a distal portion of the split piece 32 is configured to be smaller than a distal portion of the split piece 31 in thickness (diametric size) so that the split pieces 31 and 32 can be connected to each other by inserting the distal portion of the split piece 32 into the distal portion of the split piece 31.

In accordance with an exemplary embodiment, in the initial state depicted in FIG. 4, a distal portion of the puncture needle 71 is protruding from a distal-side opening of the split piece 31, and a proximal end of the split piece 31 attaches the interlock portion 72, which helps prevent the split piece 31 from obstructing the puncture needle 71 in puncturing a living body, and prevents the split piece 31 from slipping off in the extending direction of the puncture needle 71 at the time of puncturing. Similarly, a distal portion of the puncture needle 81 is exposed from a distal-side opening of the split piece 32, and a proximal end of the split piece 32 attaches the interlock portion 82, which helps prevent the split piece 32 from obstructing the puncture needle 81 in puncturing a living body, and helps prevent the split piece 32 from slipping off in the extending direction of the puncture needle 81 at the time of puncturing. Accordingly, puncturing of a living body by the puncture needles 71 and 81 and introduction of the split pieces 31 and 32 into a living body can be performed relatively smoothly.

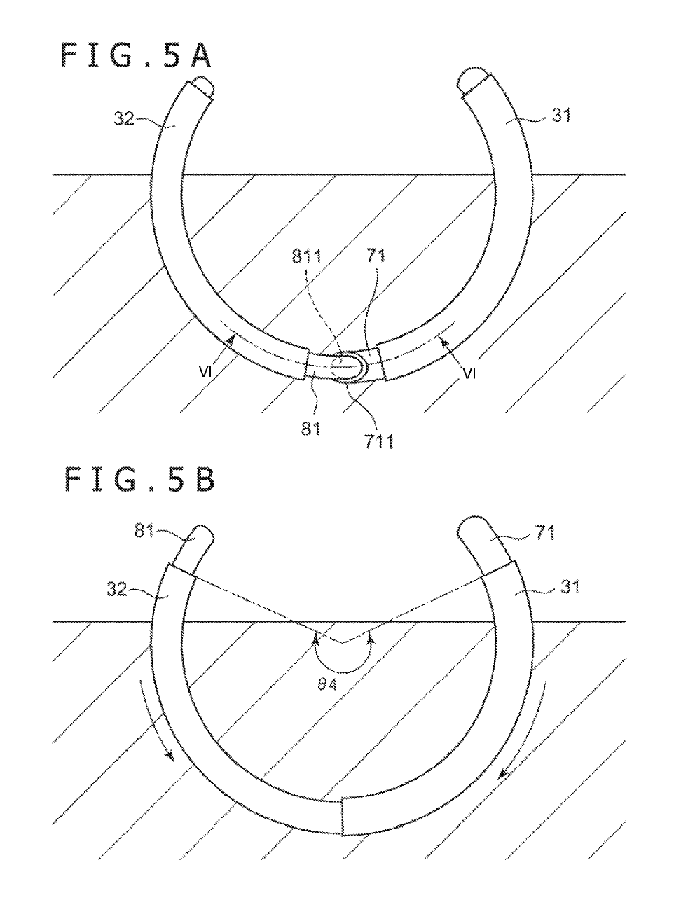

Note that the split pieces 31 and 32 cannot be connected to each other by only causing the puncture needles 71 and 81 to puncture a living body and causing the cutting edge surfaces 711 and 811 to face and overlap each other in the living body, as depicted in FIG. 5A. When the split pieces 31 and 32 (at least the split piece 31) are each further pushed in toward the distal side (the opponent side) from this condition, as depicted in FIG. 5B, the split pieces 31 and 32 can be thereby connected to each other. By such a method, the split pieces 31 and 32 can be easily connected together. In addition, the operator can judge whether or not the split pieces 31 and 32 have been connected to each other, due to the feelings obtained through the operator's hands.

Note that in the present embodiment the cutting edge surfaces 711 and 811 composed of substantially flat surfaces are provided at distal portions of the puncture needles 71 and 81 and the cutting edge surfaces 711 and 811 are configured to face and overlap each other, so that an unnecessary gap or step is unlikely formed at a boundary between the puncture needles 71 and 81. Accordingly, the connection of the split pieces 31 and 32 as aforementioned can be carried out relatively smoothly.

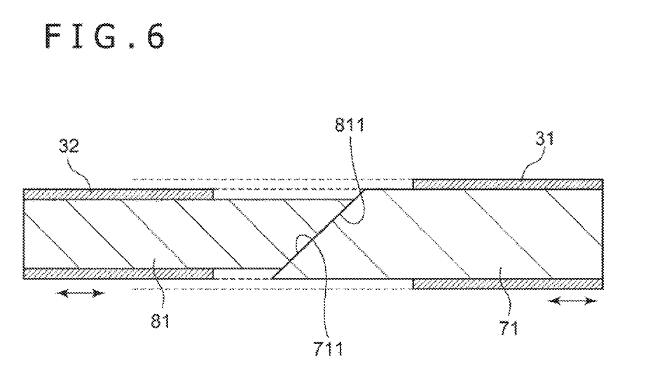

In the state where the split pieces 31 and 32 are connected together as depicted in this figure, proximal portions of the split pieces 31 and 32 are exposed outside the living body, which helps enable the implant 9 to be easily inserted into the sheath 3, as will be mentioned in the description of a procedure later. In accordance with an exemplary embodiment, as depicted in FIG. 6, since the cross-sectional shape of the split piece 32 and the cross-sectional shape of the puncture needle 71 are substantially equal, the split piece 32 and the puncture needle 71 are not liable to get caught on each other, so that the connection between the split pieces 31 and 32 can be performed relatively smoothly.

A center angle θ4 (see FIG. 5B) of the sheath 3 in the state where the split pieces 31 and 32 are connected together is not particularly limited, but is appropriately set according to various conditions. The center angle θ4 is so set that the sheath 3 can pass through the region between the urethra and the vagina and protrude to the exterior of the living body via both inguinal regions. Specifically, the center angle θ4 is preferably about 150 degrees to 270 degrees, more preferably about 170 degrees to 250 degrees, and further preferably about 190 degrees to 230 degrees.

In the present embodiment, the split pieces 31 and 32 are substantially equal in length, so that, in the state where the split pieces 31 and 32 are connected together, the boundary portion between the two (a central portion of the sheath 3) is located in the region between the urethra and the vagina.

Here, as has been described above, in the present embodiment, the shape of the puncture needle 71 is designed in conformity with the shape of the internal space of the split piece 31, and the shape of the puncture needle 81 is designed in conformity with the shape of the internal space of the split piece 32. Therefore, the split pieces 31 and 32 can be prevented from rotating relative to the puncture needles 71 and 81, and the split pieces 31 and 32 can be disposed in a predetermined posture inside a living body. In addition, since the puncture needle 71 is closing the distal-side opening of the split piece 31, a living body tissue can be restrained from entering into the internal space of the split piece 31 from the distal-side opening of the split piece 31 at the time of puncturing by the puncture needle 71. Similarly, since the puncture needle 81 is closing the distal-side opening of the split piece 32, a living body tissue can be restrained from entering into the internal space of the split piece 32 from the distal-side opening of the split piece 32 at the time of puncturing by the puncture needle 81. Accordingly, the internal space of the sheath 3 is restrained from being contaminated with the living body tissue or being clogged with the living body tissue. Consequently, the implant 9 can be smoothly placed indwelling in a living body.

The split pieces 31 and 32 configured as above are preferably light-transmitting so that the inside of the split pieces 31, 32 is visually conformed from outside, which helps ensure that the conditions (for example, the presence/absence of breakage) of the puncture needles 71 and 81 inserted in the split pieces 31 and 32 can be confirmed, which contributes to enhanced safety of the puncture apparatus 1.

Now, the shape of the sheath 3 in the state where the split pieces 31 and 32 are connected together will be described in detail below.

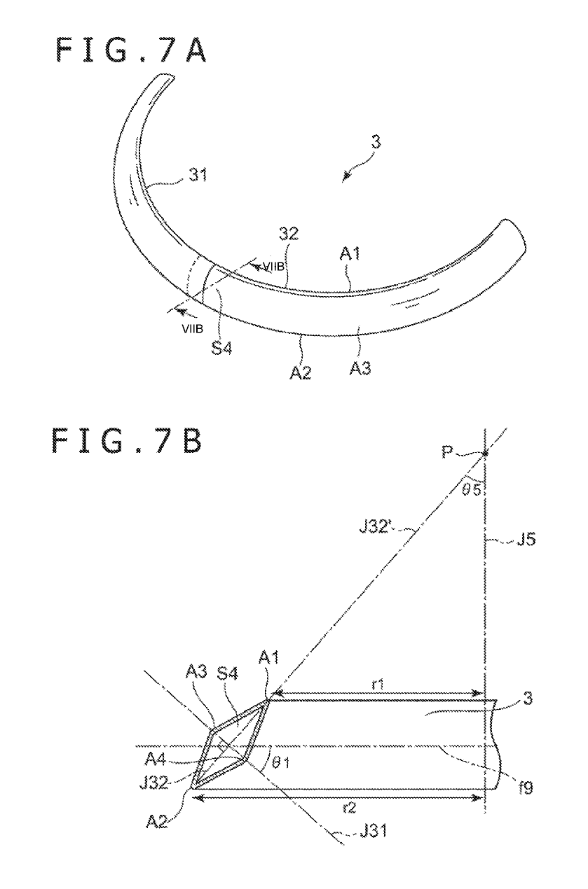

As illustrated in FIG. 7A, the sheath 3 is curved in an arcuate shape. In addition, as depicted in FIG. 7B, the sheath 3 has a flat cross-sectional shape having a minor axis J31 and a major axis J32. With the sheath 3 having a flat shape, the implant main body 91 can be controlled into a desired posture inside the sheath 3.

Note that the flat shape of the sheath 3 is not specifically restricted. Examples of the applicable flat shape can include an ellipse, a convex lens-like shape in section, a rounded-cornered rhombus, a rounded-cornered rectangle (flat shape), and a fusiform with a central portion enlarged (radially enlarged) as compared with both end portions.

The width (the length in the direction of the major axis J32) of the internal space of the sheath 3 is designed to be substantially equal to the width of the implant main body 91, which helps ensure that frictional resistance between the implant 9 and the sheath 3 is lowered, an unnecessary force is not exerted on the implant 9, and the implant main body 91 can be disposed inside the sheath 3 in a sufficiently unfolded state. Note that the width of the internal space of the sheath 3 may be shorter than the width of the implant main body 91. In this case, the width of the sheath 3 is made smaller, which contributes to lessened invasiveness.

Note that in the following, for convenience of explanation, an end portion located on an inner side in the direction of the major axis J32 of the sheath 3 as depicted in FIG. 7B will be referred to also as “inner circumferential portion A1,” an end portion located on an outer side in the direction of the major axis J32 will be referred to also as “outer circumferential portion A2,” a surface oriented to the upper side will be referred to also as “front surface A3,” and a surface oriented to the lower side will be referred to also as “back surface A4.”

When a plane containing both a center point of a circular arc of a central portion S4 of the sheath 3 and a center point of a cross-sectional shape with respect to a longitudinal direction of the sheath 3 (a plane containing a center axis of the sheath 3) is referred to as plane f9 and the angle formed between the plane f9 and the minor axis J31 of the central portion S4 is referred to as an inclination angle θ1, as depicted in FIG. 7B, then the inclination angle θ1 is preferably an acute angle. With the inclination angle θ1 set to be an acute angle, the implant main body 91 can be disposed substantially in parallel to a urethra, as will be described later. Accordingly, the urethra can be supported with enhanced effectiveness.

The inclination angle θ1 is not particularly limited, so long as it is an acute angle; for example, the inclination angle θ1 is preferably about 20 degrees to 60 degrees, more preferably about 30 degrees to 45 degrees, and further preferably about 35 degrees to 40 degrees. By this setting, the above-mentioned effects can be enhanced. Although it is preferable that the inclination angle θ1 falls within aforesaid numerical value range over the whole region in the extension direction of the sheath 3, it is sufficient that the inclination angle θ1 falls within aforesaid numerical value range at least at the central portion S4. Here, the “central portion S4” refers to a region, which can include a part located between a urethra and a vagina in the state where the sheath 3 is disposed inside a living body.

The configuration of the sheath 3 can also be expressed as follows. As illustrated in FIG. 7B, the sheath 3 can be said to be so formed that the major axis J32 is inclined against the center axis J5 of the circular arc, and that the center axis J5 and an extension line J32′ of the major axis J32 has an intersection P. In this case, an angle θ5 formed between the center axis J5 and the extension line J32′ is equal to the inclination angle θ1.

Further, the configuration of the sheath 3 can also be expressed as follows. As depicted in FIG. 7B, the sheath 3 can be said to include the inner circumferential portion A1, which is located at the inner circumferential edge thereof and has a minimum radius of curvature r1, and the outer circumferential portion A2, which is located at the outer circumferential edge thereof and has a maximum radius of curvature r2, wherein the inner circumferential portion A1 and the outer circumferential portion A2 are located with a deviation from each other in the direction of the center axis J5.

The material constituting the sheath 3 as above is preferably a rigid material such as to maintain its shape and internal space in the state where the sheath 3 is inserted in a living body. Examples of such a rigid material applicable here can include various resin materials such as polyethylene, polyimides, polyamides, polyester elastomers, polypropylene, etc. and various metallic materials such as stainless steel, aluminum or aluminum alloys, titanium or titanium alloys, etc.

Note that in addition to the adoption of a rigid material for constituting the sheath 3, adoption of a material other than a rigid material may be made while reinforcing a wall of the sheath 3 with a reinforcement member. For instance, a braiding with a high strength may be implanted in the wall of the sheath 3, whereby the sheath 3 can maintain its shape and internal space in the state of being inserted in a living body. Another example of the reinforcement member may be a spiral body implanted in the wall of the sheath 3, whereby flexibility can be ensured while maintaining an internal space to such an extent that an inserted body therein can be slid.

In addition, the sheath 3 may be provided with markers at positions which are equidistant from the central portion S4 and which are located outside of a living body when the sheath 3 is disposed in the living body, which helps ensure that by comparing the positions of both the markers, the position of the central portion S4 inside the living body can be confirmed.

The frame 2 can include a frame main body (support portion) 20, and a rotational operation unit 25 incorporated in the frame main body 20.

The frame main body 20 retains in a rotationally movable manner the puncture members 7 and 8 to which the split pieces 31 and 32 are mounted. In addition, the frame main body 20 fixes the insertion device 6 in a freely detachable manner.

The frame main body 20 has a function to determine puncture routes of the puncture needles 71 and 81 when the puncture members 7 and 8 puncture a living body tissue. Specifically, the frame main body 20 determines positional relationships of the puncture members 7 and 8, the urethral-insertion device 4, and the vaginal-insertion device 5 so as to allow the puncture needles 71 and 81 to attach each other between the urethral-insertion device 4 and the vaginal-insertion device 5 without colliding on them. In accordance with an exemplary embodiment, the frame main body 20 can restrict the positional relationships between the puncture members 7 and 8 and the urethral-insertion device 4 so that when the puncture needles 71 and 81 are rotationally moved toward their distal sides, the distal end of at least one of the puncture needles 71 and 81 (the extension line in the puncturing direction of the puncture needles 71 and 81) passes on a farther side from the axes J1 and J2 than the urethral-insertion device 4 and the distal portions of the puncture needles 71 and 81 engage each other.

As depicted in FIGS. 2 and 3, the frame main body 20 can include a bearing portion 21 bearing the shaft portions 73 and 83 of the puncture members 7 and 8, a guide portion 22 adapted to guide the puncture members 7 and 8, an interlock portion 23 interlocking the bearing portion 21 and the guide portion 22, and a fixation portion 24 adapted to fix the insertion device 6.

The bearing portion 21 is located at the proximal side of the puncture apparatus 1, and extends in a direction substantially orthogonal to the axes J1 and J2. The bearing portion 21 is formed therein with a through-hole on the axis J1, and the shaft portion 73 is inserted in the through-hole in a rotatable manner. In addition, the bearing portion 21 is formed therein with a through-hole also on the axis J2, and the shaft portion 83 is inserted in this through-hole in a rotatable manner. By this configuration, the puncture members 7 and 8 are supported on the frame main body 20 so as to be rotationally movable about the axes J1 and J2.

The guide portion 22 is located at the distal side of the puncture apparatus 1, and is disposed opposite to the bearing portion 21. The guide portion 22 is located on the inner side of the puncture members 7 and 8, has an arcuate shape extending along the puncture members 7 and 8, and is configured to support the puncture members 7 and 8 from inside. The first and second split pieces 31 and 32 are disposed at the guide portion 22 so that the back surface A4 is located on the distal side whereas the front surface A3 is located on the proximal side.

The interlock portion 23 interlocks the bearing portion 21 and the guide portion 22. In addition, the interlock portion 23 has a rod-like shape extending substantially parallel to the axes J1 and J2. The interlock portion 23 functions also as a grasping portion. By grasping the interlock portion 23, the operator can use the puncture apparatus 1 in stable conditions.

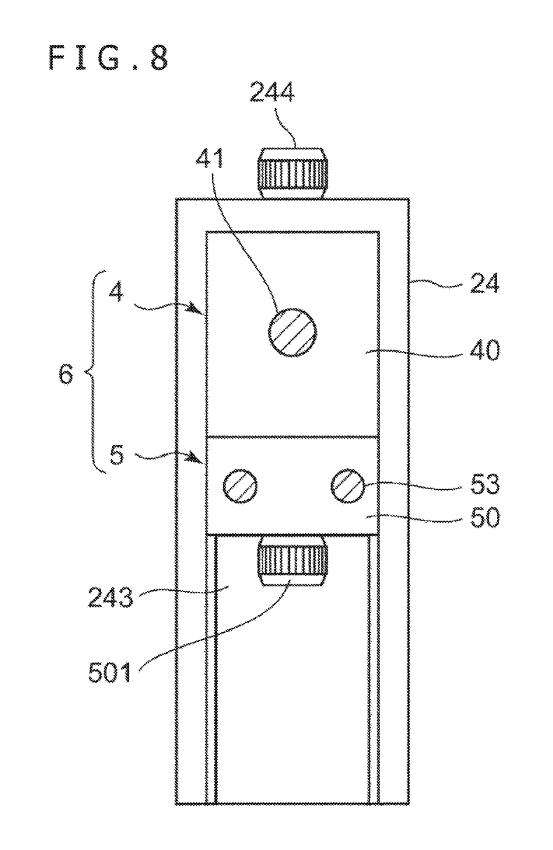

The fixation portion 24 is disposed opposite to the interlock portion 23, with the axes J1 and J2 therebetween. As depicted in FIG. 8, the fixation portion 24 can include a recess 243 into which to fit support portions 40 and 50 (described later) possessed by the insertion device 6, and a male screw 244. In such a fixation portion 24, the insertion device 6 can be fixed to the fixation portion 24 by fitting the support portions 40 and 50 into the recess 243 and, further, fastening the male screw 244 into the support portion 40.

The frame main body 20 as above has the rotational operation unit 25 incorporated therein. The rotational operation unit 25 is a mechanism for rotationally moving the puncture needles 71 and 81 in an interlocked manner, simultaneously, in opposite directions (in different directions), and by equal rotational angles. With such a rotational operation unit 25 provided, the puncture needles 71 and 81 can be operated in an interlocked manner to simultaneously puncture a living body. Therefore, the puncture apparatus 1 can be operated relatively easily, and the time taken for a procedure can be shortened. In addition, since the movements of the puncture needles 71 and 81 can be controlled in a fixed manner, the puncture apparatus 1 can be used properly and safely.

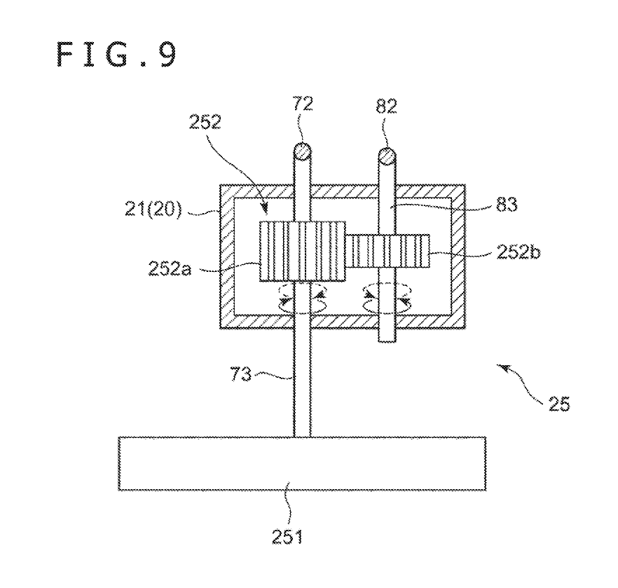

The configuration of the rotational operation unit 25 is not specifically restricted, so long as the rotational operation unit 25 can exhibit aforesaid functions. As depicted in FIG. 9, the rotational operation unit 25 in the present embodiment has a handle 251 provided on the rear side of the bearing portion 21, and the shaft portion 73 of the puncture member 7 is fixed to the handle 251. In addition, the rotational operation unit 25 has a conversion unit 252 incorporated in the bearing portion 21. The conversion unit 252 converts rotation of the handle 251 into rotary movement of the puncture needle 71 and rotary movement of the puncture needle 81. The conversion unit 252 has two gears 252a and 252b. The gear 252a is fixed to the shaft portion 73. The gear 252b is fixed to the shaft portion 83, in the state of meshing with the gear 252a. In accordance with an exemplary embodiment, the gear ratio between the gears 252a and 252b is substantially 1:1, so that the rotational angles of the gears 252a and 252b are equal. With the rotational operation unit 25 thus configured by use of gears, the rotational operation unit 25 is simple in configuration.

When the handle 251 is rotated clockwise, the puncture needle 71 is rotationally moved clockwise, and the puncture needle 81 is rotationally moved counterclockwise by the same angle as the puncture needle 71. In contrast, when the handle 251 is rotated counterclockwise, the puncture needle 71 is rotationally moved counterclockwise, and the puncture needle 81 is rotationally moved clockwise by the same angle as the puncture needle 71. Thus, according to the rotational operation unit 25, by only rotating the handle 251, the puncture needles 71 and 81 can perform simultaneous puncturing, so that the puncture apparatus 1 can be used relatively easily. Particularly, since the puncture needles 71 and 81 are rotationally moved by the same rotational angle, the movements of the puncture needles 71 and 81 are easy to expect, so that the puncture apparatus 1 can be used with enhanced safety. In addition, since the puncture needles 71 and 81 are rotationally moved by the same rotational angle, the lengths of the puncture needles 71 and 81 can be set comparatively short and equal, so that rigidity of the puncture needles 71 and 81 can be restrained from being lowered.

Note that while the handle 251 is provided in the present embodiment for manual operation of the puncture members 7 and 8, the handle 251 can be omitted in the case where a drive source such as a motor is used to operate the puncture members 7 and 8. In this case, buttons (a forward rotation button and a reverse rotation button) for controlling the drive of the motor may be disposed on the interlock portion 23, for example.

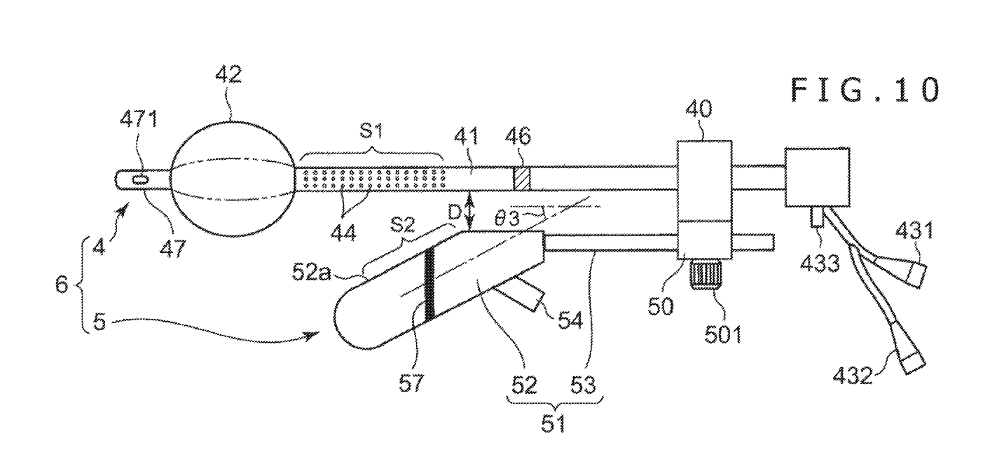

As illustrated in FIG. 10, the insertion device 6 can include the urethral-insertion device 4 and the vaginal-insertion device 5.

The urethral-insertion device 4 can include an elongated urethral-insertion portion 41 of which a part ranging from a distal end to an intermediate portion is to be inserted into a urethra, and a support portion 40 supporting the urethral-insertion portion 41. The materials constituting the urethral-insertion portion 41 and the support portion 40 are not particularly limited. Examples of the usable materials can include various metallic materials such as stainless steel, aluminum or aluminum alloys, titanium or titanium alloys, etc. and various resin materials.

The length of the urethral-insertion portion 41 (the portion on the distal side of the support portion 40) is not particularly limited but is appropriately set according to the length of the patient's urethra, the shape of the patient's bladder or the like. In view of the fact that the length of a general female urethra is about 30 mm to 50 mm, the length of the urethral-insertion portion 41 is preferably set to be about 50 mm to 100 mm, according to the fact.

The urethral-insertion portion 41 is in the form of a straight tube. The urethral-insertion portion 41 is provided at its distal portion with a balloon 42 as an expansion body having extensibility and capable of expansion and contraction, and a urine drainage portion 47.

The balloon 42 is so disposed as to be located inside a bladder when the urethral-insertion portion 41 is inserted in a urethra. The balloon 42 is connected through the inside of the urethral-insertion portion 41 to a balloon port 431 provided at a proximal portion of the urethral-insertion portion 41. A balloon expanding device such as a syringe can be connected to the balloon port 431. When a working fluid (a liquid such as physiological saline solution, or a gas or the like) is supplied from the balloon expanding device into the balloon 42, the balloon 42 is expanded. When the working fluid is drawn out of the balloon 42 by the balloon expanding device, the balloon 42 is contracted. Note that in FIG. 10, a contracted state of the balloon 42 is indicated by alternate long and two short dashes line, while an expanded state of the balloon 42 is indicated by solid line.

The urine drainage portion 47 is used to drain urine present in the bladder in a state where the urethral-insertion portion 41 is inserted in the urethra. The urine drainage portion 47 is provided with a urine drain hole 471 providing communication between the inside and the outside of the urine drainage portion 47. The urine drain hole 471 is connected through the inside of the urethral-insertion portion 41 to a urine drain port 432 provided at a proximal portion of the urethral-insertion portion 41. Therefore, urine introduced via the urine drain hole 471 can be drained through the urine drain port 432.

The balloon 42 and the urine drainage portion 47 can be configured, for example, by a double lumen.

The urethral-insertion portion 41 is provided at its intermediate portion with a marker 46 for confirmation of the depth of insertion of the urethral-insertion portion 41 into a urethra. The marker 46 is located at a urethral orifice when the urethral-insertion portion 41 is inserted in a urethra and the balloon 42 is located inside a bladder. This structure can help enable the depth of insertion of the urethral-insertion portion 41 into the urethra to be easily confirmed. Note that the marker 46 is required only to be visually recognizable externally, and may be composed, for example, of a colored part, a rugged (projected and recessed) part or the like. Alternatively, in place of the marker 46, graduations accompanied by numerical values of distance from the distal end of the urethral-insertion portion 41 may be provided.

In addition, the urethral-insertion portion 41 is formed in its intermediate portion (on the distal side of the marker 46) with a plurality of suction holes 44. The plurality of suction holes 44 are arranged over the whole region in the circumferential direction of the urethral-insertion portion 41. Each of the suction holes 44 is connected through the inside of the urethral-insertion portion 41 to a suction port 433 provided in the support portion 40. A suction device such as a pump can be connected to the suction port 433. When the suction device is operated in a state where the urethral-insertion portion 41 is inserted in a urethra, a urethral wall can be attracted and fixed by suction onto the suction holes 44. Particularly, with the plurality of suction holes 44 provided over the entire region in the circumferential direction of the urethral-insertion portion 41 as in this embodiment, a broad range of the urethral wall can be attracted and fixed by suction onto the urethral-insertion portion 41.

When the urethral-insertion portion 41 is pushed in toward the inner side of a body (toward the distal side of the urethral-insertion portion 41) in a state where the urethral wall is attracted and fixed by suction onto the urethral-insertion portion 41, the urethra and the bladder are accordingly pushed in toward the inner side of the body, whereby the bladder can be shifted to such a position as not to overlap with a puncture route of the puncture needles 71 and 81. Therefore, the puncture route of the puncture needles 71 and 81 can be secured in a larger form, resulting in that puncturing by the puncture needles 71 and 81 can be performed relatively accurately and safely.

Note that the number of the suction holes 44 is not particularly limited. For example, only one suction hole 44 may be provided. In addition, the layout of the suction holes 44 is not specifically restricted. For example, the suction holes 44 may be formed in only a part in the circumferential direction of the urethral-insertion portion 41.

Now, inclinations of the urethral-insertion portion 41 and the puncture needles 71 and 81 will be described. As illustrated in FIG. 3, the rotational axes J1 and J2 of the puncture needles 71 and 81 are inclined against the axis J3 of the urethral-insertion portion 41 so that the separated distance between the rotational axes J1 and J2 and the axis J3 of the urethral-insertion portion 41 increases along the distal direction. The angle formed between the axes J1 and J2 and the axis J3, for example, the inclination angle θ2 of a plane f9 (plane f1) in relation to a plane f2 orthogonal to the axis J3 of the urethral-insertion portion 41, is not particularly limited, but is preferably about 20 degrees to 60 degrees, more preferably about 30 degrees to 45 degrees, and further preferably about 35 degrees to 40 degrees. As a result of this, puncturing by the puncture needles 71 and 81 can be performed easily, and the puncture distances of the puncture needles 71 and 81 can be made shorter.

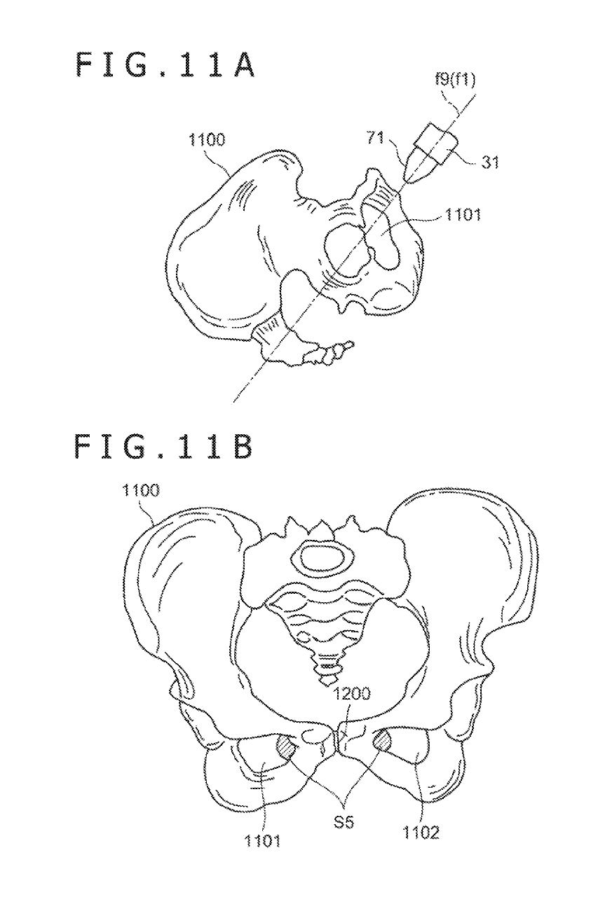

In accordance with an exemplary embodiment, when the inclination angle θ2 is set within the above-mentioned range, as depicted in FIG. 11A, it is possible for the puncture needle 71 to widely capture an obturator foramen 1101 on one side of a pelvis 1100 on a planar basis and it is possible to widely secure a puncture space for the puncture needle 71. Similarly, it is possible for the puncture needle 81 to widely capture an obturator foramen 1102 on the other side of the pelvis 1100 on a planar basis and it is possible to widely secure a puncture space for the puncture needle 81. In accordance with an exemplary embodiment, with the patient set in a predetermined body position (lithotomy position), the puncture needles 71 and 81 can be made to puncture in directions comparatively nearer to the directions perpendicular to the obturator foramens 1101 and 1102. Consequently, puncturing by the puncture needles 71 and 81 can be carried out relatively easily.

When the puncture needles 71 and 81 puncture a living body tissue in directions comparatively nearer to the directions perpendicular to the obturator foramens 1101 and 1102, the puncture needles 71 and 81 pass through shallow portions of the tissue, so that the distal ends of the puncture needles 71 and 81 pass between the obturator foramens 1101 and 1102 by passing shorter distances. Therefore, as depicted in FIG. 11B, the puncture needles 71 and 81 can be made to pass through the obturator foramens 1101 and 1102 while passing near a pubic symphysis 1200, preferably through safety zones S5. The safety zone S5 is a zone where there are few nerves and blood vessels to which the damage is desired to be avoided. When the puncture needles 71 and 81 are made to pass through the safety zones S5, therefore, puncturing by the puncture needles 71 and 81 can be performed safely. Consequently, invasiveness can be lowered, and the burden on the patient is lessened.

Thus, by setting the inclination angle θ2 to within the above-mentioned range, puncturing of a patient by the puncture needles 71 and 81 can be carried out. In addition, by puncturing at the above-mentioned angle, a tissue between a mid-urethra (which refers to a middle portion in the lengthwise direction of the urethra) and the vagina can be easily aimed at as a target. Here, the position between the mid-urethra and the vagina is a position suitable as a part in which to embed the implant 9 for treatment of urinary incontinence. Taking the tissue between the mid-urethra and the vagina as a target, therefore, a more effective treatment can be carried out.

Note that in the case where the inclination angle θ2 is below the above-mentioned lower limit or above the above-mentioned upper limit, a problem may arise depending on individual differences in the patient, the posture during a procedure, or the like factors. For instance, it may be impossible for the puncture needles 71 and 81 to widely capture the obturator foramens 1101 and 1102 on a planar basis, or it may be impossible to sufficiently shorten the puncture route of the puncture needles 71 and 81.

While in the puncture apparatus 1 the urethral-insertion device 4 is fixed to the frame 2 so that the inclination angle θ2 is constant, this is not restrictive, and the inclination angle θ2 may be variable. Where the inclination angle θ2 is variable, the inclination angle θ2 can be controlled according to the patient, which enhances usability of the puncture apparatus 1. In addition, the rotational axes J1 and J2 of the puncture needles 71 and 81 may be inclined against the axis J3 of the urethral-insertion portion 41 so that the separated distance between the rotational axes J1 and J2 and the axis J3 of the urethral-insertion portion 41 decreases along the distal direction.

As illustrated in FIG. 10, the vaginal-insertion device 5 can include an elongated vaginal-insertion portion 51 of which a part ranging from a distal end to an intermediate portion is to be inserted in a vagina, and a support portion 50 supporting the vaginal-insertion portion 51. In addition, the vaginal-insertion portion 51 can include a distal portion 52 located on a distal side, and a shaft portion 53 connected to a proximal portion of the distal portion 52, the shaft portion 53 being supported by the support portion 50.

In accordance with an exemplary embodiment, the support portion 50 is provided with a male screw 501. With the male screw 501 fastened to a female screw (not depicted) of the support portion 40, the vaginal-insertion device 5 can be fixed to the urethral-insertion device 4.

The materials constituting the vaginal-insertion portion 51 and the support portion 50 are not specifically restricted. Examples of the applicable materials can include various metallic materials such as stainless steel, aluminum or aluminum alloys, titanium or titanium alloys, etc. and various resin materials, like in the case of the urethral-insertion device 4 (the urethral-insertion portion 41 and the support portion 40).

The distal portion 52 can be elongated in shape. In addition, the distal portion 52 has an upper surface (a surface on the urethral-insertion portion 41 side) 52a inclined against the urethral-insertion portion 41 so as to be spaced more from the urethral-insertion portion 41 as the distance from the distal end decreases. This structure can help ensure that the positional relationship between the urethral-insertion portion 41 and the distal portion 52 can be made closer to the actual positional relationship between a urethra and a vagina. Therefore, in the mounted state, the puncture apparatus 1 is held onto the patient more stably, and the burden on the patient is mitigated. In addition, a wide space for puncturing by the puncture needles 71 and 81 can be secured between the distal portion 52 and the urethral-insertion portion 41, so that it is possible to perform relatively safe puncturing by the puncture needles 71 and 81.

The inclination angle θ3 of the upper surface 52a in relation to the urethral-insertion portion 41 is not particularly limited. For example, the inclination angle θ3 is preferably about 0 degree to 45 degrees, and more particularly about 0 degree to 30 degrees, which setting helps enable the above-mentioned effects to be exhibited more remarkably. In the case where the inclination angle θ3 is below the above-mentioned lower limit or above the above-mentioned upper limit, on the other hand, a problem may arise depending on individual differences in the patient, the posture during a procedure or the like factors. For example, the vagina or the urethra may be deformed unnaturally in the mounted state, possibly resulting in that the puncture apparatus 1 cannot be held stably.

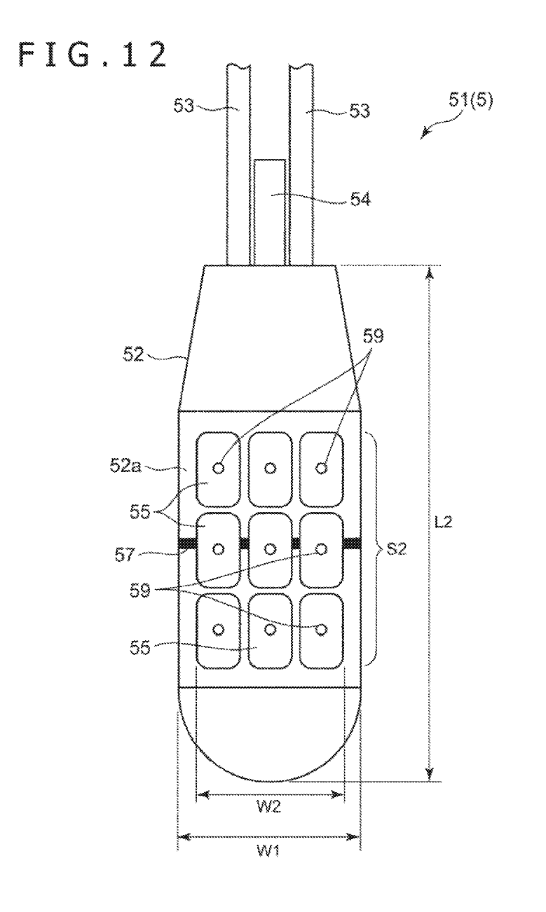

The distal portion 52 has a flat shape of being collapsed in the vertical direction of the puncture apparatus 1 (in the direction in which the urethra and the vagina are arrayed). In addition, as depicted in FIG. 12, the distal portion 52 can include a central part having a substantially constant width, and a distal part, which is rounded in shape. The length L2 of the distal portion 52 is not particularly limited, and is preferably about 20 mm to 100 mm, more preferably about 30 mm to 60 mm. The width W1 of the distal portion 52 is not specifically restricted, and is preferably about 10 mm to 50 mm, more preferably about 20 mm to 40 mm. The thickness of the distal portion 52 is not particularly limited, and is preferably about 5 mm to 25 mm, more preferably about 10 mm to 20 mm. When the length, width and thickness of the distal portion 52 are set to within such ranges, the distal portion 52 has a shape and size suitable for general vaginas. As a result, stability of the puncture apparatus 1 in the mounted state can be enhanced, and the burden on the patient can be lessened.

In addition, as illustrated in FIG. 12, the upper surface 52a of the distal portion 52 is formed with a plurality of bottomed recesses 55. Each recess 55 is provided at its bottom surface with a suction hole 59. Each of such suction holes 59 is connected through the inside of the distal portion 52 to a suction port 54 provided at a proximal part of the distal portion 52. The suction port 54 is so provided as to be located in the exterior of a living body in the mounted state. A suction device such as a pump can be connected to the suction port 54. When the suction device is operated in a state where the distal portion 52 is inserted in a vagina, a vaginal anterior wall is attracted and fixed by suction onto the distal portion 52. When the vaginal-insertion portion 51 is pushed in toward the inner side of the body (toward the distal side of the vaginal-insertion portion 51) in a state where the vaginal anterior wall is attracted and fixed by suction onto the distal portion 52, the vagina can be pushed in accordingly. Therefore, the layout and shape of the vagina can be adjusted, so that a puncture routes for the puncture needles 71 and 81 can be secured sufficiently. As a result, puncturing by the puncture needles 71 and 81 can be performed relatively accurately and safely.

Note that the number of the recesses 55 is not particularly limited. For example, only one recess 55 may be provided.

As depicted in FIG. 10, a region S2 where the recesses 55 are formed faces a region S1 where the suction holes 44 are formed. The puncture apparatus 1 can be configured so that the distal ends of the puncture needles 71 and 81 pass between these regions S1 and S2. As has been described above, the urethral wall is attracted by suction onto the urethral-insertion portion 41 in the region S1, and the vaginal wall is attracted by suction onto the distal portion 52 in the region S2. Therefore, between the regions S1 and S2, the urethral wall and the vaginal wall are spaced apart from each other more widely and more assuredly. By passing the puncture needles 71 and 81 through such a zone, puncturing by the puncture needles 71 and 81 can be performed safely.

Note that, as depicted in FIG. 12, the region S2 preferably extends over substantially the entire range in the width direction of the upper surface 52a. The width W2 of the region S2 is not particularly limited, and is preferably about 9 mm to 49 mm, more preferably about 19 mm to 39 mm, which helps ensure that the vaginal wall can be attracted by suction onto the distal portion 52 more assuredly, without being much affected by the shape of each patient's vaginal wall.

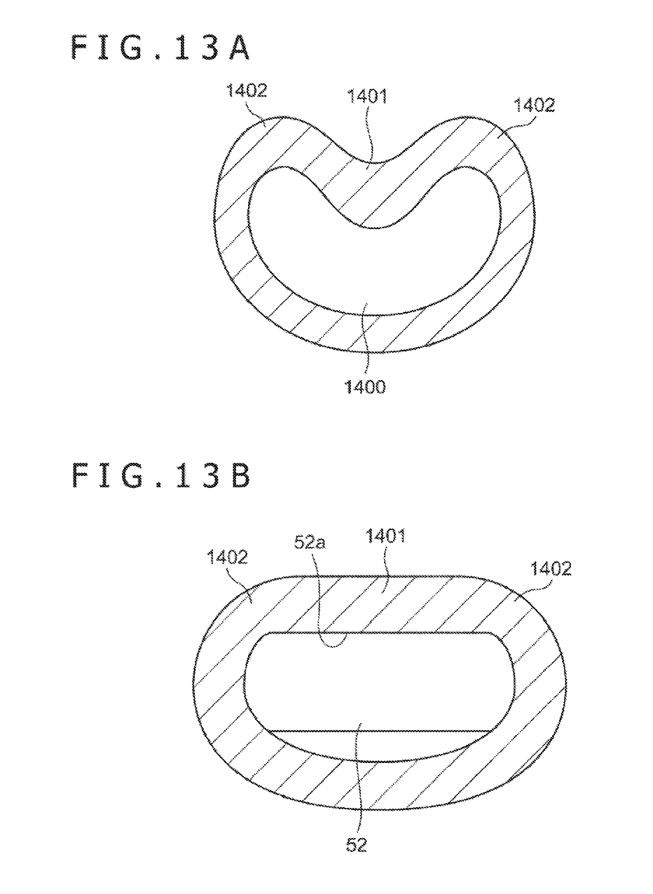

Particularly, some patients may have a vagina 1400 in which a central portion 1401 of a vaginal anterior wall droops down into the vagina, as depicted in FIG. 13A, to form hollowed portions (portions also called “button holes”) 1402 on both sides of the central portion 1401. Even in such a case, not only the central portion 1401 but also the hollowed portions 1402 can be attracted by suction with enhanced reliability, as depicted in FIG. 13B. With the hollowed portions 1402 thus attracted by suction onto the distal portion 52 more assuredly, the hollowed portions 1402 can be brought away from a urethra 1300, so that puncturing the hollowed portions 1402 by the puncture needles 71 and 81 by mistake can be prevented more effectively.

In addition, the distal portion 52 is provided with a marker 57 which helps enable the puncture route of the puncture needles 71 and 81 to be confirmed. The marker 57 is so provided that the puncture needles 71 and 81 puncture a tissue between the vaginal wall located over the marker 57 and the urethral wall. By confirming the position of the marker 57, therefore, the puncture route of the puncture needles 71 and 81 can be easily confirmed, and operability and safety of the puncture apparatus 1 are enhanced. The marker 57 is preferably provided at least on a lower surface of the distal portion 52. The lower surface is oriented toward the vaginal orifice in the inserted state, and is a surface, which can be visually checked by the operator by way of the vaginal orifice. With the marker 57 provided on the lower surface, therefore, the puncture route of the puncture needles 71 and 81 can be confirmed relatively assuredly. The marker 57 is required only to be visually recognizable externally, and can be configured, for example, by a colored part, a rugged (projected and recessed) part, or the like.

The separated distance D between the distal portion 52 configured in this manner and the urethral-insertion portion 41 is not particularly limited. The separated distance D is preferably set to be about 5 mm to 40 mm, in accordance with the separated distance between a urethral orifice and a vaginal orifice in general females.

The shaft portion 53 is in the form of a thin (diametrically small-sized) bar extending substantially in parallel to the urethral-insertion portion 41. The length of the shaft portion 53 (the separated distance between the distal portion 52 and the support portion 50) is not specifically restricted, and is preferably not more than about 100 mm, more preferably in the range of about 20 mm to 50 mm. By such a setting, the shaft portion 53 can be appropriate in length, whereby operability of the puncture apparatus 1 can be enhanced. If the length of the shaft portion 53 exceeds the above-mentioned upper limit, a problem may arise depending on the configuration of the frame 2 or the like factors. For example, the center of gravity of the puncture apparatus 1 may be spaced from the patient so largely that the stability of the puncture apparatus 1 in the mounted state is lowered.

The insertion device 6 has thus been described above. Note that the insertion device 6 in this embodiment has been so configured that the urethral-insertion device 4 and the vaginal-insertion device 5 are freely detachable, but this is not restrictive. For example, the urethral-insertion device 4 and the vaginal-insertion device 5 may not be freely detachable. While the urethral-insertion portion 41 is fixed to the support portion 40, this is not restrictive, and the urethral-insertion portion 41 may be slidable in the axial direction thereof relative to the support portion 40. In this case, a configuration may be adopted wherein, for example, when a predetermined male screw provided on the support portion 40 is untightened, the urethral-insertion portion 41 becomes slidable relative to the support portion 40, and when the screw is tightened, the urethral-insertion portion 41 becomes fixed to the support portion 40. According to such a configuration, the length of the urethral-insertion portion 41 (the length on the distal side of the support portion 40) can be adjusted, which enhances usability of the insertion device 6. This applies also to the vaginal insertion portion 51.

Now, a method of using the puncture apparatus 1, or a method of embedding the implant 9 into a living body by use of the puncture apparatus 1 will be described below.

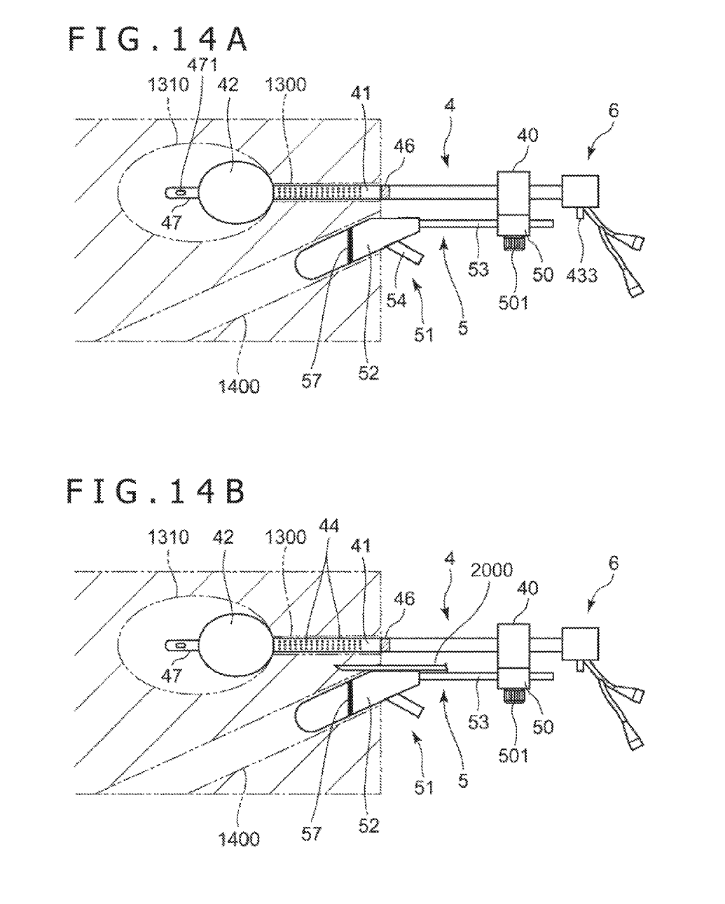

First, a patient is set into a lithotomy position on an operating table, and the insertion device 6 is mounted to the patient, as depicted in FIG. 14A.

Specifically, first, the urethral-insertion portion 41 of the urethral-insertion device 4 is inserted into the urethra 1300. In this instance, the depth of insertion can be confirmed by observing the marker 46, and the balloon 42 is disposed inside a bladder 1310. The urethra 1300 is rectified into a predetermined shape (rectilinear shape) by the urethral-insertion portion 41. Next, the balloon 42 is expanded, and urine is drained from the bladder 1310 through the urine drain hole 471 as required. The vaginal-insertion portion 51 of the vaginal-insertion device 5 is inserted into the vagina 1400. Then, the puncture route of the puncture needles 71 and 81 is confirmed by use of the marker 57, after which the support portion 50 is fixed to the support portion 40. This completes mounting of the insertion device 6 to the patient.

Next, suction devices are connected to the suction ports 433 and 54. The suction devices are operated to attract the urethral wall onto the urethral-insertion portion 41 by suction, and to attract the vaginal anterior wall onto the vaginal-insertion portion 51 by suction. For example, when the urethral wall is properly attracted onto the urethral-insertion portion 41 by suction, the suction holes 44 are closed with the urethral wall, so that suction through the suction port 433 is stopped or weakened. Similarly, when the vaginal anterior wall is properly attracted onto the vaginal-insertion portion 51 by suction, the suction holes 59 are closed with the vaginal wall, so that suction through the suction port 54 is stopped or weakened. Therefore, on the basis of the conditions of suction through the suction ports 433 and 54 (for example, the magnitude of sounds generated by suction), the operator can check whether or not the urethral wall and the vaginal anterior wall have been properly attracted onto the urethral-insertion portion 41 and the vaginal-insertion portion 51 by suction.

Note that the insertion device 6 may have a confirmation mechanism for mechanically confirming the attracted-by-suction state. The confirmation mechanism is not specifically restricted, so long as the mechanism enables the attracted-by-suction state to be confirmed. For instance, the confirmation mechanism may include a flow rate measurement unit (negative pressure gauge) for measuring flow rates through the suction ports 433 and 54, and a determination unit for determining whether or not the attraction by suction has been properly performed, on the basis of the measurement results supplied from the flow rate measurement unit.

Subsequently, liquid dissection is conducted as required. Specifically, as illustrated in FIG. 14B, a puncture needle of a syringe 2000 is made to puncture the vaginal anterior wall from between the urethral-insertion portion 41 and the vaginal-insertion portion 51, and a liquid such as physiological saline solution, a local anesthetic, etc. is injected into a living body tissue between the urethra 1300 and the vagina 1400 (between the regions S1 and S2). This causes the living body tissue between the regions S1 and S2 to swell, whereby the urethral wall is pressed against the urethral-insertion portion 41, and the vaginal anterior wall is pressed against the vaginal-insertion portion 51.

Here, suction via the suction holes 44 and 59 is preferably continued also during the liquid dissection. When the urethral wall is pressed against the urethral-insertion portion 41 due to the liquid dissection, the suction from the suction port 433 is stopped or weakened. Similarly, when the vaginal anterior wall is pressed against the vaginal-insertion portion 51, the suction from the suction port 54 is stopped or weakened. On the basis of the conditions of suction from the suction ports 433 and 54, therefore, the operator can confirm whether or not the liquid dissection has been properly conducted.

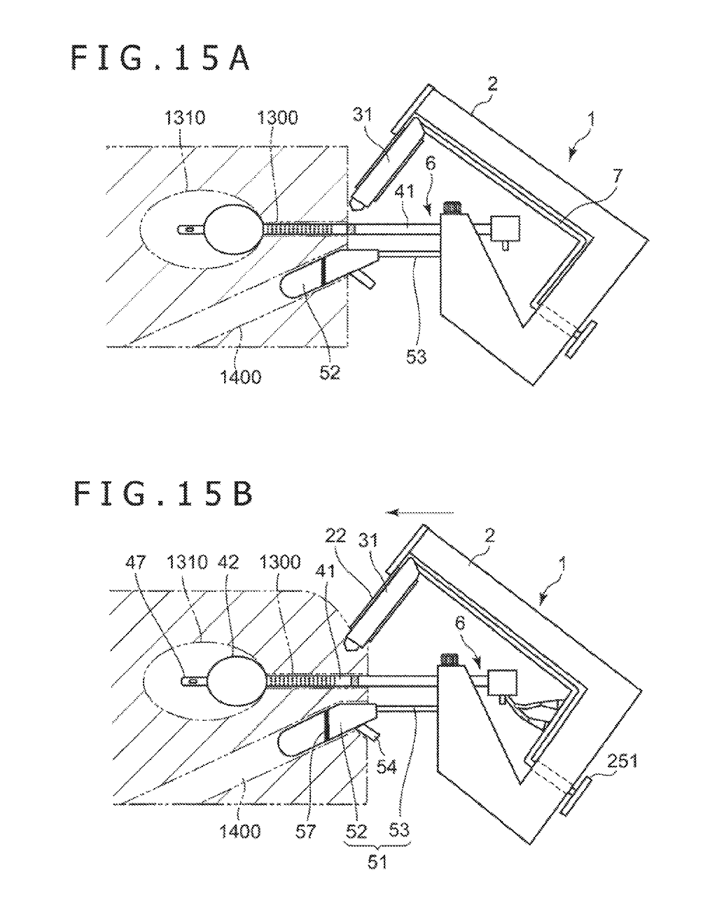

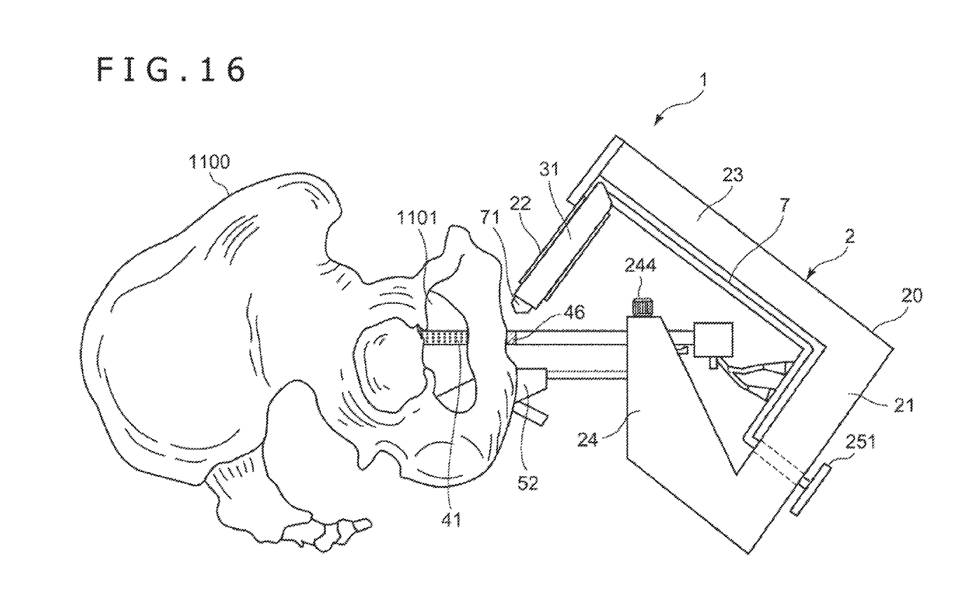

After the liquid dissection is performed to put the urethral wall and the vaginal anterior wall into a sufficiently spaced apart state, the frame 2 is fixed to the insertion device 6, as depicted in FIG. 15A. This results in a state where the puncture apparatus 1 is mounted to the patient. In this state, the positional relationship between the pelvis 1100 and the puncture apparatus 1 is as illustrated in FIG. 16.

Then, as depicted in FIG. 15B, the puncture apparatus 1 is pushed in toward the inner side of the body. As has been described above, the urethral wall is attracted onto the urethral-insertion portion 41 by suction, and the vaginal wall is attracted onto the vaginal-insertion portion 51 by suction. When the puncture apparatus 1 is pushed in toward the inner side of the body, therefore, the urethra 1300 and the vagina 1400 are pushed in as well, so that the tissue therebetween is stretched. Therefore, sagging of the tissue can be reduced, and puncturing of the tissue by the puncture needles 71 and 81 is made easier to carry out.

Next, in the state where the puncture apparatus 1 is pushed in toward the inner side of the body, the puncture apparatus 1 is so positioned that the puncture route of the puncture needles 71 and 81 passes through the safety zones S5 in the left and right obturator foramens 1101 and 1102, and, maintaining this condition, the handle 251 is rotated.

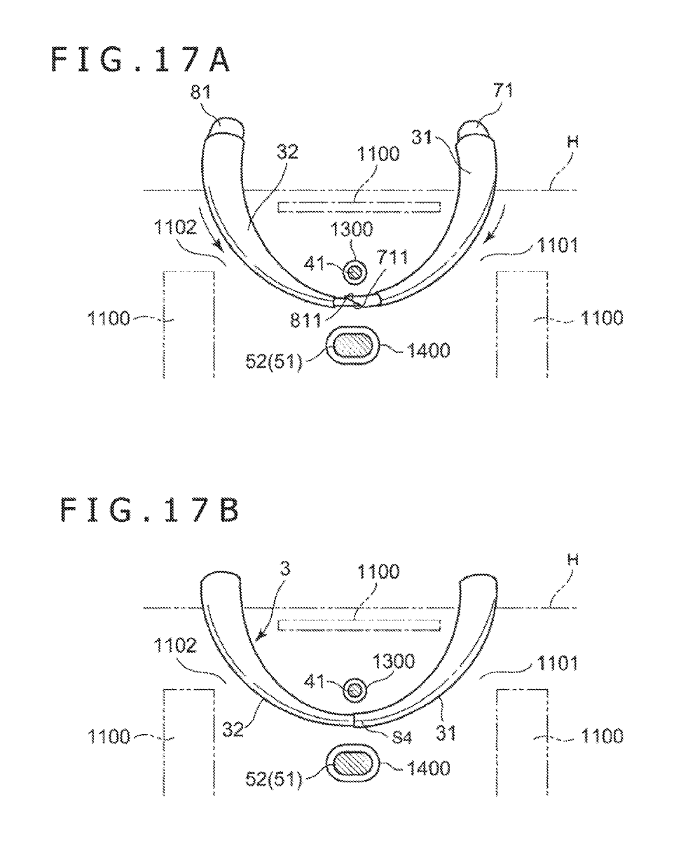

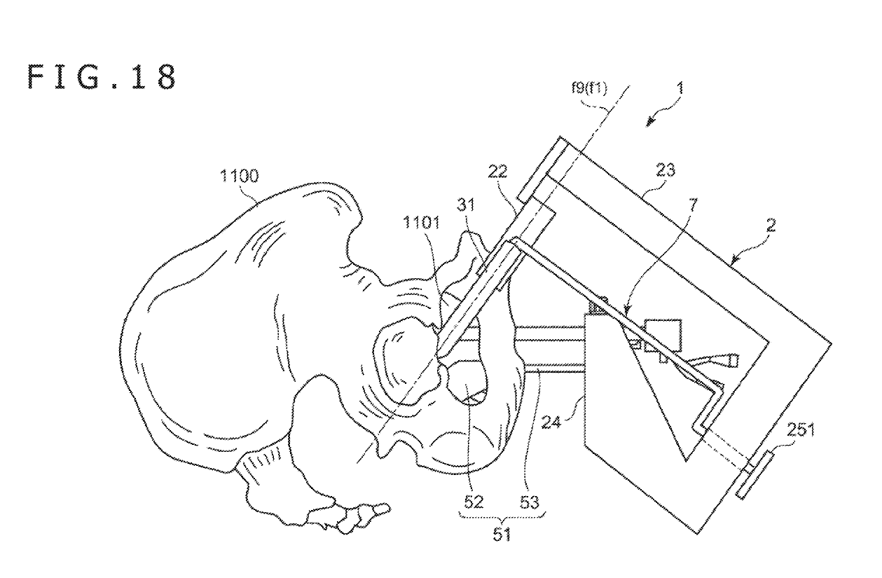

As a result, as depicted in FIG. 17A, the puncture needle 71 enters the body by puncturing a body surface H (first part) of the patient's right-side inguinal region or neighboring part, and passes through the obturator foramen 1101, to reach a region between the urethra 1300 and the vagina 1400. Simultaneously, the puncture needle 81 enters the body by puncturing a body surface H (second part) of the patient's left-side inguinal region or neighboring part, and passes through the obturator foramen 1102, to reach a region between the urethra 1300 and the vagina 1400. Then, at a predetermined position in the vicinity of the region between the urethra 1300 and the vagina 1400, the distal portions of the puncture needles 71 and 81 contact each other, and the cutting edge surfaces 711 and 811 face and overlap each other. In this instance, as has been described above, the puncture needles 71 and 81 are made to puncture the living body tissue substantially perpendicularly to the obturator foramens 1101 and 1102, whereby a passage can be formed at a position suitable for the implant 9 to be placed indwelling. In this state, the positional relationship between the pelvis 1100 and the puncture apparatus 1 is as illustrated in FIG. 18.

Subsequently, the split pieces 31 and 32 are both pushed in toward the inner side of the living body, and are connected together. As a result, the sheath 3 is disposed in the living body. Next, the handle 251 is operated to rotationally move the puncture needles 71 and 81 in the direction reverse to that at the time of puncturing, whereby only the puncture needles 71 and 81 are drawn out of the living body, leaving the sheath 3 in the living body. Subsequently, the frame 2 is detached from the insertion device 6. This results in a state where only the sheath 3 is disposed in the living body, as depicted in FIG. 17B. The sheath 3 is disposed in the living body with both end openings of the sheath 3 exposed in the exterior of the living body.

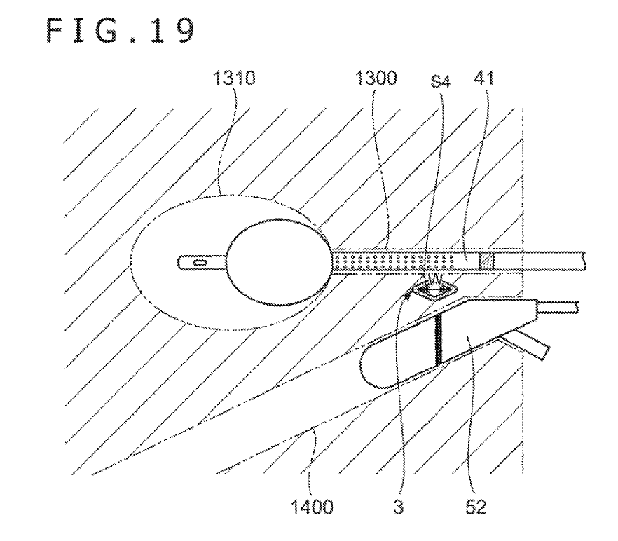

Next, the position of the sheath 3 is regulated as required. Specifically, the left and right lengths of protrusion of the sheath 3 are equalized, and the central portion S4 of the sheath 3 is thereby positioned between the urethra 1300 and the vagina 1400. In this state, as depicted in FIG. 19, the central portion S4 of the sheath 3 is disposed with its width direction (major axis J32 direction) W substantially in parallel to the urethra 1300. In accordance with an exemplary embodiment, the urethra 1300 rectified by the urethral-insertion portion 41 inserted therein and the width direction W of the central portion S4 of the sheath 3 can be substantially parallel to each other.

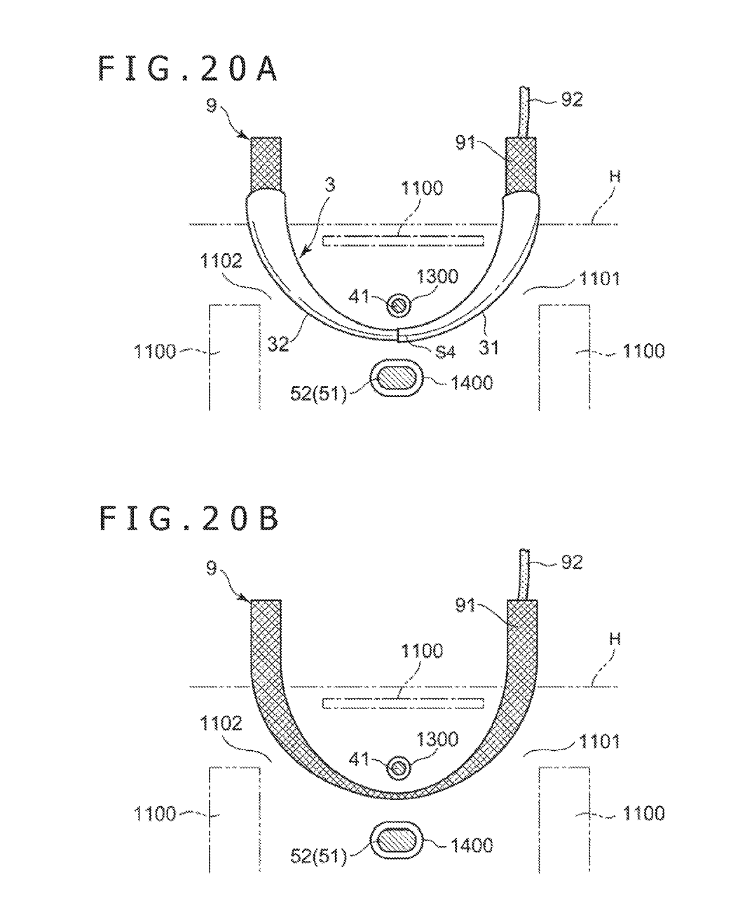

Subsequently, while taking the implant 9 out of the wrapping material 90, the implant 9 is inserted into the sheath 3 to put the implant main body 91 into the state of partly protruding through both end openings of the sheath 3, as depicted in FIG. 20A. The implant 9 is thus kept wrapped in the wrapping material 90 until immediately before disposed into the sheath 3, whereby the implant 9 can be prevented from contamination. Note that since the sheath 3 is flat in shape as has been described above, the posture of the implant main body 91 follows the flat shape of the sheath 3, so that the implant main body 91 is disposed in the sheath 3 with its width direction coinciding with the width direction of the sheath 3. As for relationship with the urethra 1300, the implant main body 91 is disposed in parallel to the urethra 1300 rectified in shape.

Next, the split pieces 31 and 32 are both pulled toward their proximal sides, to be disconnected from each other and to be draw out of the living body. In this case, the split pieces 31 and 32 are substantially simultaneously moved in opposite directions, and the split pieces 31 and 32 are moved in arcuate paths in conformity with their respective shapes, which helps enable the sheath 3 to be smoothly removed from the living body.

When the split pieces 31 and 32 are gradually removed from the living body as aforementioned, the surrounding tissues having been pushed open by the sheath 3 return to their original positions, and the tissues come into contact with the implant main body 91 gradually in the order from a central portion toward both end portions of the implant main body 91. As has been described above, the split pieces 31 and 32 are moved in the directions along their shapes, and the sheath 3 has an internal space in which the implant main body 91 can be moved under sufficiently low friction. This enables the implant main body 91 to be placed indwelling as it is, without any unnecessary tension exerted thereon. Consequently, it is unnecessary to adjust the tension on the implant main body 91. As a result of the above operations, the implant main body 91 is left embedded in the living body, as depicted in FIG. 20B.

In the state where the implant 9 is embedded in the living body, the implant main body 91 is disposed in the region between the urethra 1300 and the vagina 1400 substantially in parallel to the urethra 1300. Therefore, the urethra 1300 can be supported by the implant main body 91 over a wider region.

Thus, by removing the sheath 3 from the living body by dividing the sheath 3, the sheath 3 can be drawn out of the living body relatively easily. In addition, the split pieces 31 and 32 being drawn out exert little influence on the posture of the implant main body 91 in the region between the urethra 1300 and the vagina 1400. Therefore, the implant main body 91 can be embedded in a desired posture.

In accordance with an exemplary embodiment, since the split pieces 31 and 32 are drawn out of the living body with the urethral-insertion portion 41 inserted in the urethra 1300, excessive tension can be prevented from being exerted on the urethra 1300 by the implant main body 91 placed indwelling in the living body.

Subsequently, the insertion device 6 is drawn out of the living body. Specifically, the urethral-insertion portion 41 is drawn out of the urethra 1300, and the vaginal-insertion portion 51 is drawn out of the vagina 1400. After the urethral-insertion device 4 is drawn out, the urethra 1300 returns into its shape in a natural state. Since the implant main body 91 is kept embedded in the living body tissue, however, the urethra 1300 in the natural state and the implant main body 91 can be maintained in the mutually parallel state.

Thereafter, unnecessary portions of the implant main body 91 are cut away, and the procedure is finished.

As has been described above, according to the puncture apparatus 1, placing the implant 9 indwelling can be coped with only a minimally invasive procedure such as puncturing by the puncture needles 71 and 81, without need to perform a heavily invasive procedure such as incision. Therefore, the burden on the patient is relatively light. Besides, safety for the patient is relatively high. Particularly, puncturing a living body from both sides by use of the two puncture needles 71 and 81 help enable the puncture needles 71 and 81 to be designed to be shorter as compared with the case where the living body is punctured from one side by use of a single puncture needle. Accordingly, the bending or twisting of the puncture needles 71 and 81 due to the resistance received at the time of puncturing can be restrained. Consequently, puncturing by the puncture needles 71 and 81 can be performed with enhanced accuracy and with enhanced safety.

In addition, since the implant main body 91 can be embedded in parallel to the urethra 1300, the urethra 1300 can be supported over a wider region. In accordance with an exemplary embodiment, since the puncture needles 71 and 81 can puncture the living body while avoiding the urethra 1300 and the vagina 1400, puncturing of the urethra 1300 or the vagina 1400 by the puncture needles 71 and 81 by mistake can be prevented, whereby safety is relatively ensured. In addition, the problems arising from incision of the vagina as in the related art can be avoided, such as the problem in which the implant 9 is exposed to the inside of the vagina from the wound caused by the incision, or the problem in which complications such as infection from the wound are generated. Thus, the procedure is highly safe, and the implant 9 can be reliably embedded.

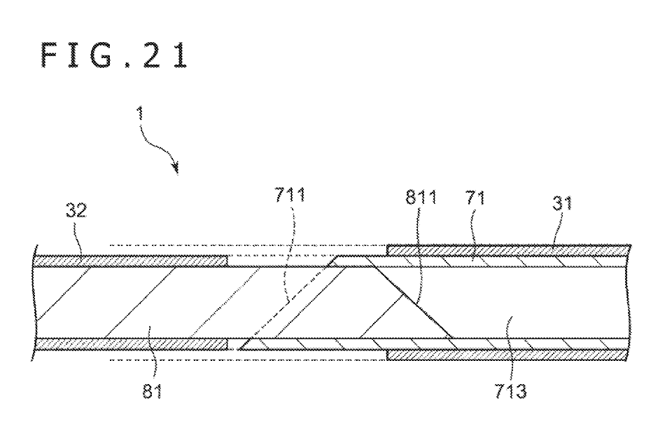

FIG. 21 is a sectional view illustrating first and second puncture needles possessed by a puncture apparatus according to a second embodiment of the present disclosure, depicting a mutually engaged state of the first and second puncture needles. Note that FIG. 21 is a sectional view corresponding to FIG. 6 of the first embodiment described above.

Referring to FIG. 21, the second embodiment of the puncture apparatus will be described below. The following description will focus on differences of the second embodiment from the first embodiment above, and the description of the same items as above will be omitted.

The second embodiment is the same as the first embodiment except for a difference in the configuration of the first puncture needle.

As illustrated in FIG. 21, a puncture needle 71 in this embodiment is tube shaped, and has a through-hole 713 opening to a distal end (cutting edge surface 711) and a proximal end. When the puncture needle 71 and a puncture needle 81 are rotationally moved toward their distal sides, a distal portion of the puncture needle 81 is inserted into the through-hole 713 via the distal-side opening of the puncture needle 71. With the distal portion of the puncture needle 81 thus inserted in a distal portion of the puncture needle 71, the puncture needles 71 and 81 can be engaged with each other in a stable state with less positional deviation. Therefore, connection between split pieces 31 and 32 can be performed more smoothly.

Particularly, in this embodiment, the cutting edge surface 711 of the puncture needle 71 and a cutting edge surface 811 of the puncture needle 81 are oriented in the same direction (upward in FIG. 21). Since the puncture needle 81 is inserted into the puncture needle 71 gradually starting from its distal end, therefore, the puncture needle 81 is inserted into the puncture needle 71 more easily as compared, for example, with the case where the cutting edge surfaces 711 and 811 face each other as in the aforementioned first embodiment, which helps enhance operability of the puncture apparatus 1.

Note that while the puncture needle 71 is tube shaped in this embodiment, the configuration of the puncture needle 71 is not restricted to this, so long as the puncture needle 81 can be inserted into a distal portion of the puncture needle 71. For example, only a distal portion may be tube shaped, with a proximal portion being solid.

According to the second embodiment, also, equivalent or similar effects to those of the first embodiment above can be produced.

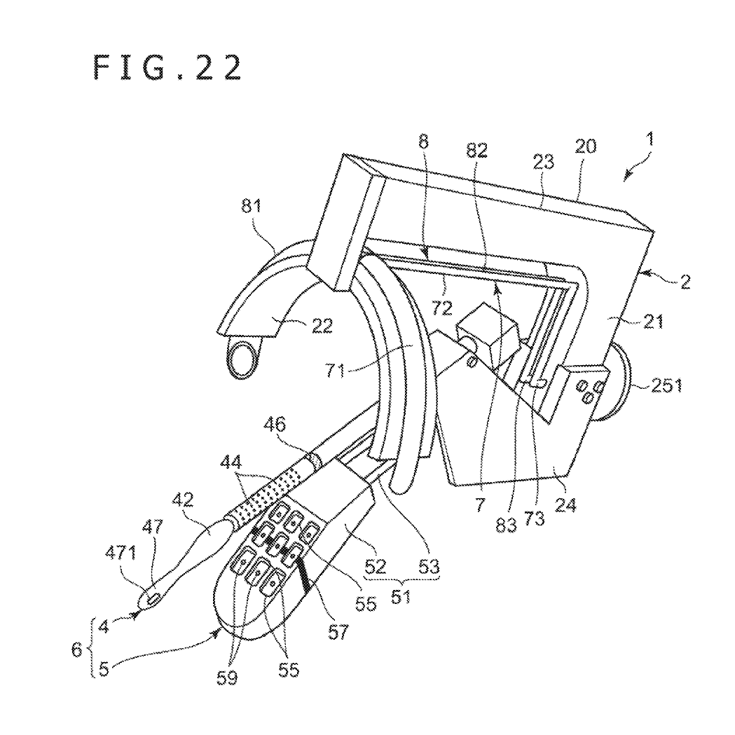

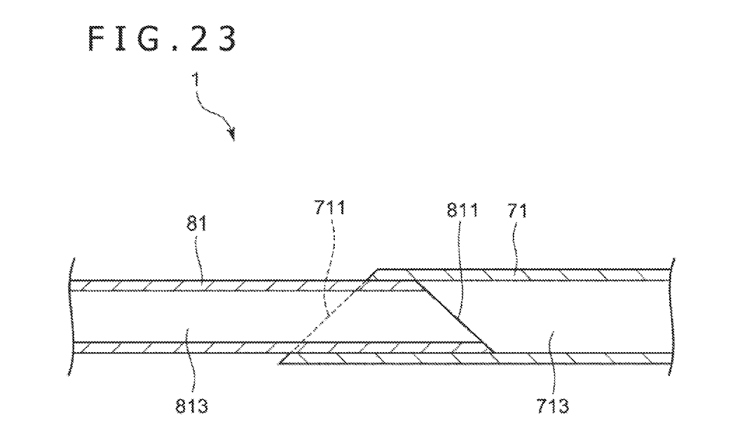

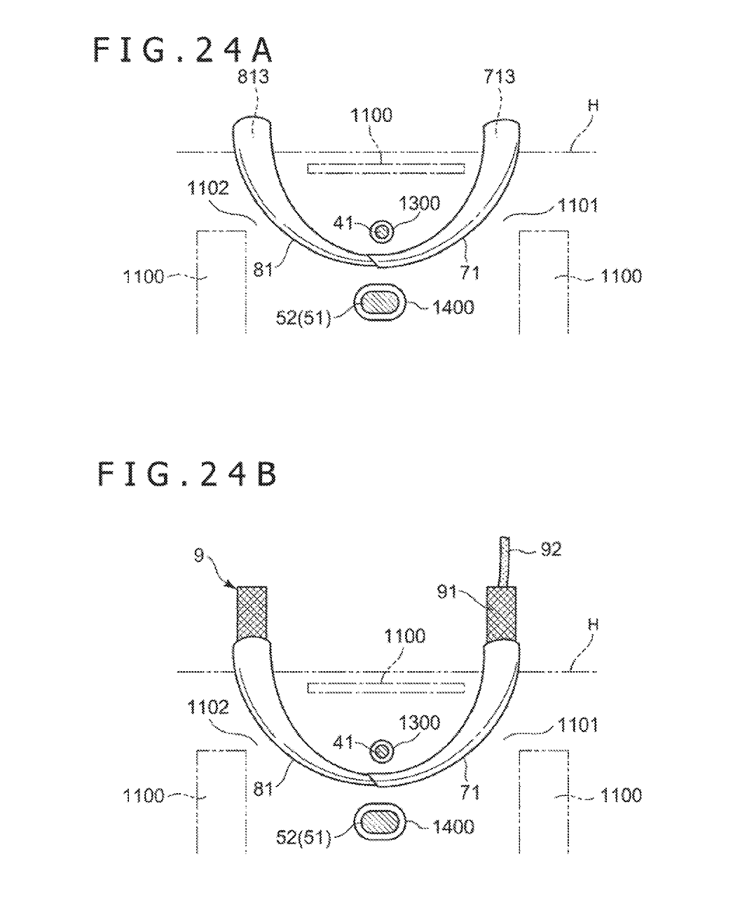

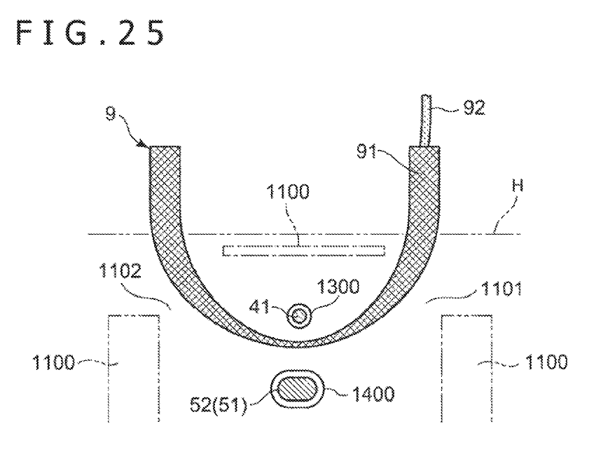

FIG. 22 is a perspective view illustrating a puncture apparatus according to a third embodiment of the present disclosure; FIG. 23 is a sectional view illustrating first and second puncture needles possessed by the puncture apparatus of FIG. 22, depicting the first and second puncture needles in their mutually engaged state; FIGS. 24A and 24B are views for explaining an operating procedure of the puncture apparatus of FIG. 22; and FIG. 25 is a view for explaining the operating procedure of the puncture apparatus of FIG. 22. Note that FIG. 23 is a sectional view corresponding to FIG. 6 of the first embodiment above.

Referring to these drawings, the third embodiment of the puncture apparatus will now be described below. The following description will focus on differences of the third embodiment from the aforementioned embodiments, and the description of the same items as above will be omitted.

This third embodiment is the same as the first embodiment above except for a difference in the configuration of the first and second puncture needles and for omission of the sheath.

As depicted in FIG. 22, in a puncture apparatus 1 according to this third embodiment, the sheath 3, or the split pieces 31 and 32 are omitted.

As depicted in FIG. 23, puncture needles 71 and 81 are each tube shaped. Therefore, the puncture needle 71 is formed therein with a through-hole (internal space) 713 opening to a distal end (cutting edge surface 711) and a proximal end. The puncture needle 81 is formed therein with a through-hole (internal space) 813 opening to a distal end (cutting edge surface 811) and a proximal end. When the puncture needles 71 and 81 are rotationally moved toward their distal sides, a distal portion of the puncture needle 81 is inserted into the through-hole 713 via a distal-side opening of the puncture needle 71. With the distal portion of the puncture needle 81 thus inserted in the puncture needle 71, the puncture needles 71 and 81 can be engaged with each other in a stable state with less positional deviation.

Particularly, in this embodiment, the cutting edge surfaces 711 and 811 of the puncture needles 71 and 81 are oriented in the same direction (upward in FIG. 23). Therefore, the puncture needle 81 is inserted into the puncture needle 71 gradually starting from its distal end, so that the puncture needle 81 can be inserted into the puncture needle 71 more easily as compared, for example, with the case where the cutting edge surfaces 711 and 811 face each other as in the first embodiment above. This contributes to enhanced operability of the puncture apparatus 1.

When the puncture needles 71 and 81 are engaged with each other in the above-mentioned manner, the through-holes 713 and 813 communicate with each other to form a space into which to insert an implant 9. In accordance with an exemplary embodiment, in this embodiment, the puncture needles 71 and 81 function also as the sheath 3 in the first and second embodiments described above. Since the puncture needles 71 and 81 serve also as the sheath 3, the implant 9 can be embedded into a living body more smoothly.

Now, a method of embedding the implant 9 into a living body by use of the puncture apparatus 1 according to this embodiment will be described.

First, through the same step as in the first embodiment above, the puncture needles 71 and 81 are made to puncture a living body, and a distal portion of the puncture needle 81 is inserted into a distal portion of the puncture needle 71 to make the puncture needles 71 and 81 engage each other, as depicted in FIG. 24A. As a result of this operation, the through-holes 713 and 813 communicate with each other. Next, the implant 9 is inserted into the puncture needles 71 and 81, to result that an implant main body 91 protrudes from both end openings of the puncture needles 71 and 81, as depicted in FIG. 24B. Subsequently, the puncture needles 71 and 81 are rotationally moved toward their proximal sides, and both the puncture needles 71 and 81 are thereby drawn out of the living body, leaving the implant main body 91 in the living body. As a result of these operations, the implant main body 91 is left embedded in the living body, as depicted in FIG. 25.

According to the third embodiment, also, equivalent or similar effects to those of the first embodiment as aforementioned can be produced.

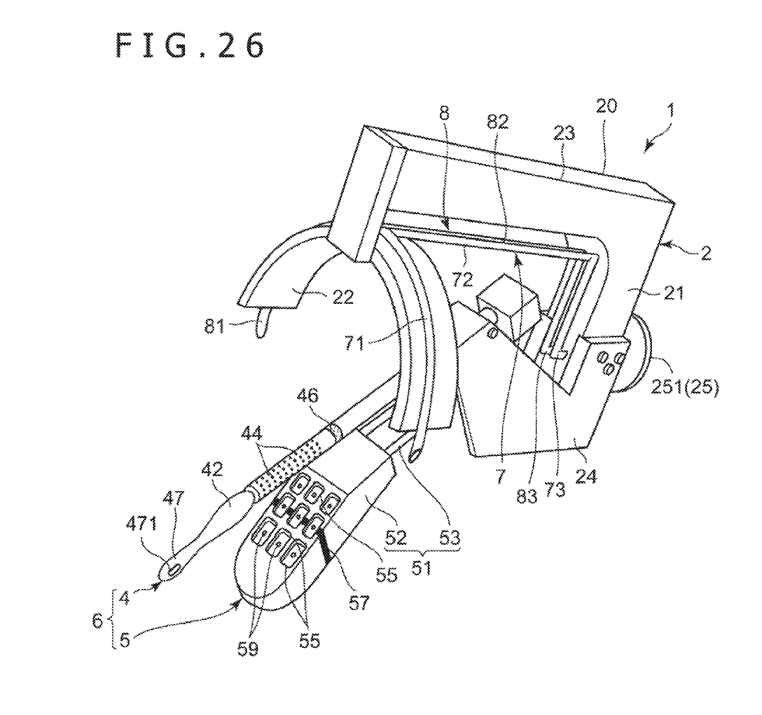

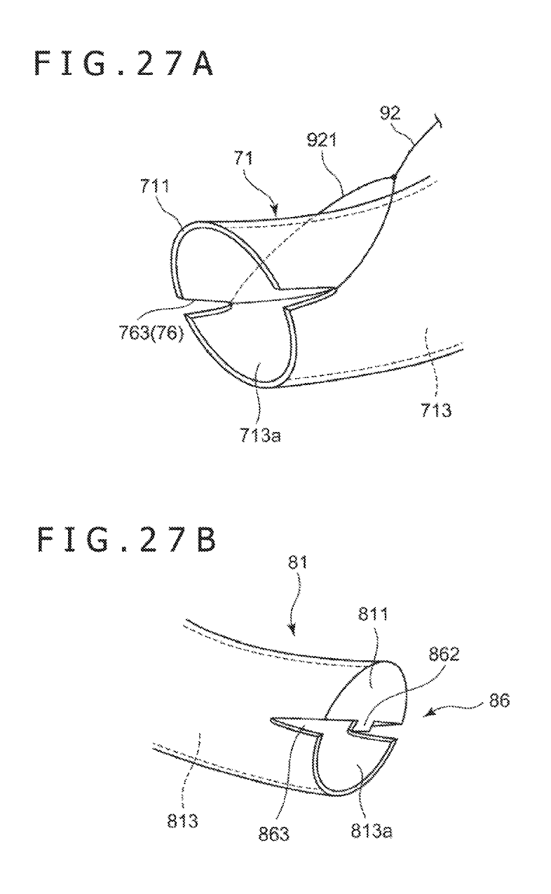

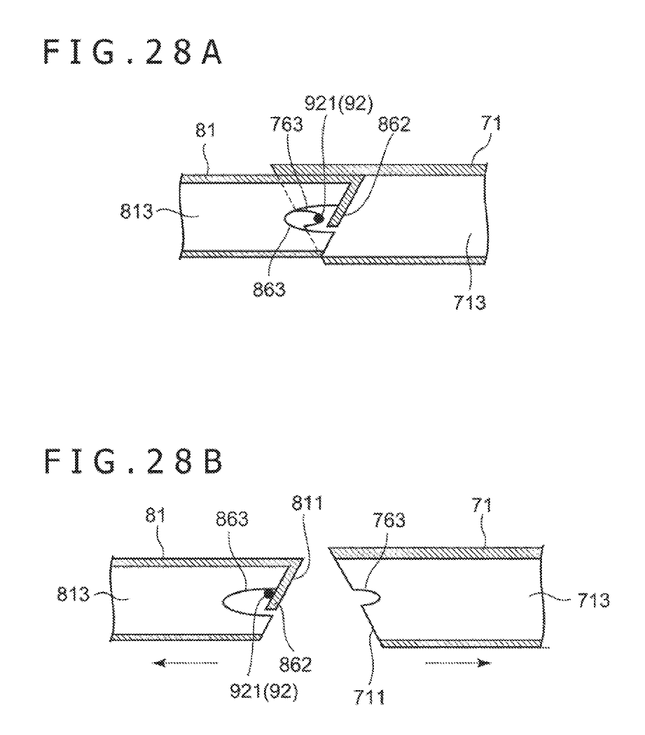

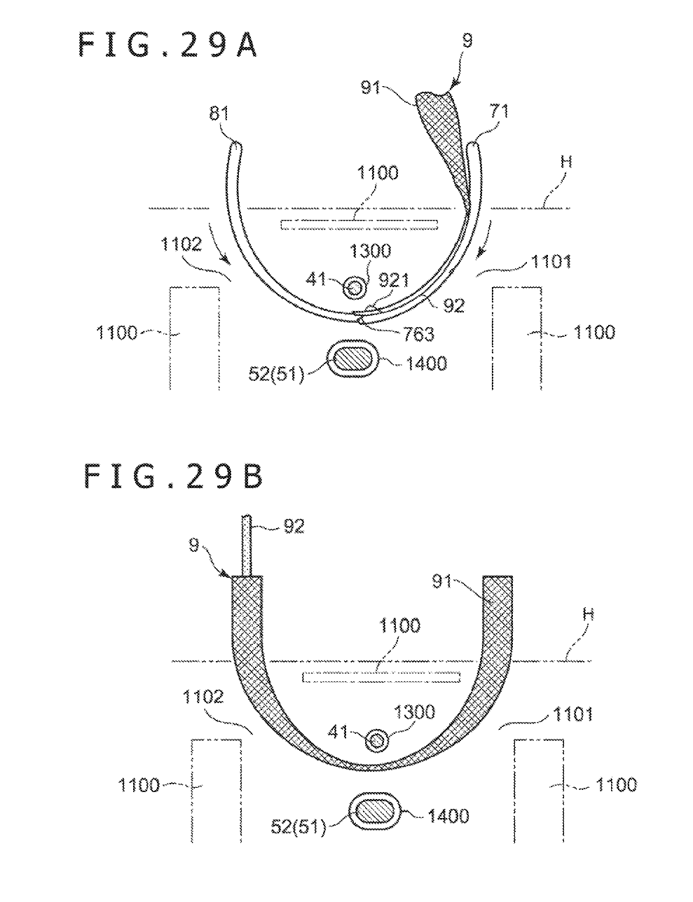

FIG. 26 is a perspective view illustrating a puncture apparatus according to a fourth embodiment of the present disclosure. FIG. 27A is a perspective view depicting a distal portion of a first puncture needle, and FIG. 27B is a perspective view illustrating a distal portion of a second puncture needle. FIGS. 28A and 28B are sectional views for explaining transfer of an implant from the first puncture needle to the second puncture needle. FIGS. 29A and 29B are views for explaining an operating procedure of the puncture apparatus depicted in FIG. 26.

Referring to these drawings, the fourth embodiment of the puncture apparatus will now be described below. The following description will focus on differences of the fourth embodiment from the aforementioned embodiments, and the description of the same items as above will be omitted.

This fourth embodiment is the same as the first embodiment above except for a difference in the configuration of the first and second puncture needles and for omission of the sheath.

As illustrated in FIG. 26, in a puncture apparatus 1 according to this fourth embodiment, the sheath 3, or the split pieces 31 and 32 are omitted.

As depicted in FIGS. 27A and 27B, puncture needles 71 and 81 are each tube shaped. Therefore, the puncture needle 71 is formed therein with a through-hole 713 opening to a distal end (cutting edge surface 711) and a proximal end. The puncture needle 81 is formed therein with a through-hole 813 opening to a distal end (cutting edge surface 811) and a proximal end. When the puncture needles 71 and 81 are rotationally moved toward their distal sides, a distal portion of the puncture needle 81 is inserted into the through-hole 713 from a distal-side opening of the puncture needle 71. With the distal portion of the puncture needle 81 thus inserted in the puncture needle 71, the puncture needles 71 and 81 can be engaged with each other in a state with less positional deviation.

Particularly, in this embodiment, the cutting edge surfaces 711 and 811 of the puncture needles 71 and 81 are oriented in the same direction. Therefore, the puncture needle 81 is inserted into the puncture needle 71 gradually starting from its distal end, so that the puncture needle 81 can be inserted into the puncture needle 71 easily as compared, for example, with the case where the cutting edge surfaces 711 and 811 face each other as in the aforementioned first embodiment.

In accordance with an exemplary embodiment, as illustrated in FIG. 27A, the puncture needle 71 is provided at its distal portion with an implant retaining portion 76 for retaining an implant 9. The implant retaining portion 76 has a groove 763 crossing a distal-side opening 713a of the through-hole 713. A belt 92 of the implant 9 is engaged in the groove 763, whereby the implant 9 can be retained on the puncture needle 71. In this case, it is recommendable to tie the belt 92 so as to form a loop 921, and engage the loop 921 in the groove 763, which helps enable the implant 9 from coming off the puncture needle 71. In addition, since the loop 921 is disposed crossing the distal-side opening 713a, the transfer of the implant 9 to the puncture needle 81 as described later can be reliably performed.

In accordance with an exemplary embodiment, as depicted in FIG. 27B, the puncture needle 81 is provided at its distal portion with an implant receiving portion 86 for receiving the implant 9 retained by the puncture needle 71. The implant receiving portion 86 can include a groove 863 crossing a distal-side opening 813a of the through-hole 813, and a claw portion 862 projecting into the groove 863. The claw portion 862 projects at an inclination to the proximal side, like a so-called “barb.” Besides, the claw portion 862 is formed as a part of the cutting edge surface 811.

When the distal portion of the puncture needle 81 is inserted into the puncture needle 71, the claw portion 862 passes by the loop 921, and the loop 921 enters into the groove 863, as depicted in FIG. 28A. Thereafter, when the puncture needle 81 is drawn out of the puncture needle 71, the loop 921 is caught on the claw portion 862, as depicted in FIG. 28B, so that the implant 9 is transferred to the puncture needle 81. Thus, the distal portions of the puncture needles 71 and 81 engage (are connected with) each other, which engagement connects the implant receiving portion 86 to the implant 9, and the implant 9 is detached from the implant retaining portion 76 upon separation of the implant retaining portion 76 and the implant receiving portion 86 from each other. According to such a configuration, in the process of puncturing by the puncture needles 71 and 81 and drawing-out of the puncture needles 71 and 81, the transfer of the implant 9 from the puncture needle 71 to the puncture needle 81 can be performed automatically. As a result, the procedure conducted using the puncture apparatus 1 can be carried out relatively easily and smoothly.

Now, a method of embedding the implant 9 into a living body by use of the puncture apparatus 1 according to this fourth embodiment will be described.

First, through the same step as in the aforementioned first embodiment, the puncture needles 71 and 81 are put into a state before puncturing a living body. Next, the implant 9 is engaged in the groove 763 of the puncture needle 71, after which the puncture needles 71 and 81 are rotationally moved toward their distal sides, to puncture the living body and to insert a distal portion of the puncture needle 81 into a distal portion of the puncture needle 71, as depicted in FIG. 29A. In this instance, the implant 9 is guided into the living body together with the puncture needle 71, starting from its distal side, and is transferred to the puncture needle 81. Subsequently, the puncture needles 71 and 81 are rotationally moved toward their proximal sides, and are thereby drawn out of the living body. In this instance, the implant 9 is discharged to the exterior of the living body together with the puncture needle 81, starting from its distal side. As a result, an implant main body 91 is left embedded in the living body, as illustrated in FIG. 29B. According to such a method, the implant 9 can be embedded into a living body with a reduced number of procedure steps as compared, for example, with a method in which a sheath is used as in the aforementioned first embodiment.

According to the fourth embodiment, also, equivalent or similar effects to those of the aforementioned first embodiment can be produced.

Note that the external shapes of the puncture needles 71 and 81 in this fourth embodiment may be equivalent or similar to those in the first to third embodiments described above. This enables the implant 9 to be embedded into a living body in an unfolded state and in a proper posture. In addition, the implant 9 may be disposed in the through-hole 713 of the puncture needle 71, in the state of being retained by the puncture needle 71. In addition, while the puncture needle 71 has the implant retaining portion 76 and the puncture needle 81 has the implant receiving portion 86 in this fourth embodiment, in contrast, the puncture needle 71 may have the implant receiving portion 86 and the puncture needle 81 may have the implant retaining portion 76.

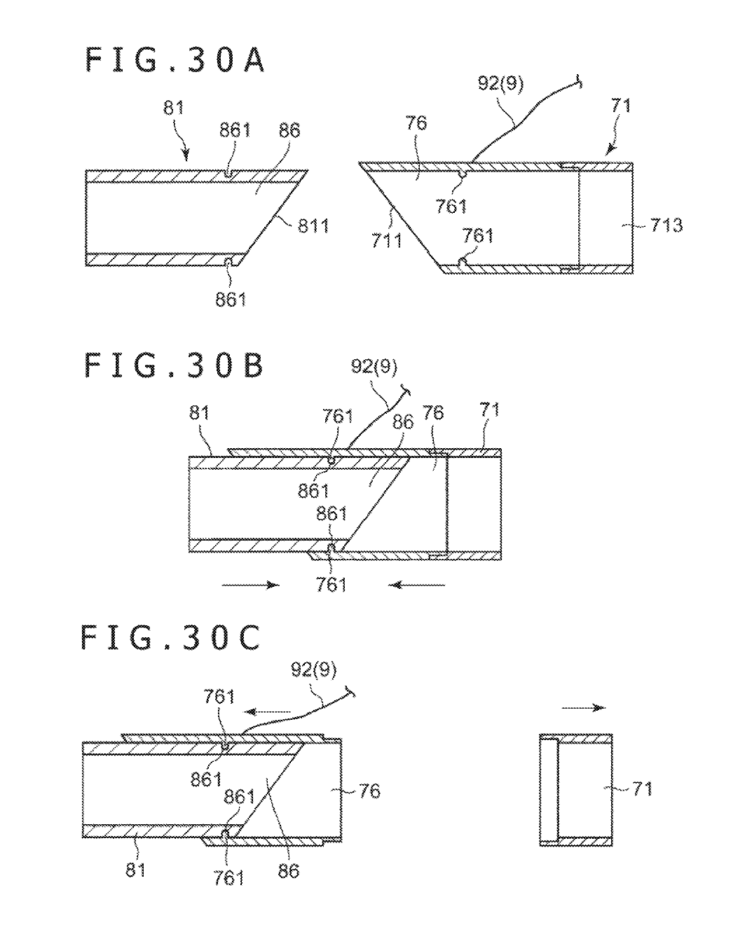

FIG. 30A is a sectional view illustrating first and second puncture needles possessed by a puncture apparatus according to a fifth embodiment of the present disclosure, and FIGS. 30B and 30C are sectional views for explaining a transfer of an implant from the first puncture needle to the second puncture needle.

Referring to the drawing, the fifth embodiment of the puncture apparatus will be described below. The following description will focus on differences of this fifth embodiment from the aforementioned embodiments, and the description of the same items as above will be omitted.

The fifth embodiment is the same as the fourth embodiment above except for a difference in the configuration of the first and second puncture needles.

As illustrated in FIG. 30A, in a puncture apparatus 1 in this embodiment, an implant retaining portion 76 is detachably provided at a distal portion of a puncture needle 71. In addition, the implant retaining portion 76 is provided with a projection 761 at an inner circumferential surface thereof. In accordance with an exemplary embodiment, an implant receiving portion 86 is provided at a distal portion of a puncture needle 81. The implant receiving portion 86 has a recess 861 provided in an outer circumference of the puncture needle 81.

As depicted in FIG. 30B, the projection 761 and the recess 861 are engaged with each other by insertion of a distal portion (implant receiving portion 86) of the puncture needle 81 into a distal portion (implant retaining portion 76) of the puncture needle 71. Upon engagement between the projection 761 and the recess 861, the implant retaining portion 76 is fixed to the implant receiving portion 86, and an implant 9 is transferred to the puncture needle 81 together with the implant retaining portion 76.

Then, as depicted in FIG. 30C, the puncture needle 81 is drawn out of the puncture needle 71, whereupon a distal portion of the implant 9 is discharged to the exterior of a living body together with the puncture needle 81. According to such a configuration, in the process of puncturing by the puncture needles 71 and 81 and drawing-out of the puncture needles 71 and 81, the transfer of the implant 9 from the puncture needle 71 to the puncture needle 81 can be performed automatically. Therefore, the procedure conducted using the puncture apparatus 1 can be carried out relatively easily and smoothly.

According to the fifth embodiment, also, equivalent or similar effects to those of the aforementioned first embodiment can be produced.

Note that while the implant retaining portion 76 and the implant receiving portion 86 are fixed to each other by projection-recess fit in this fifth embodiment, the fixing method is not restricted to this. For example, press fit, adhesion or the like may also be used for the fixation.

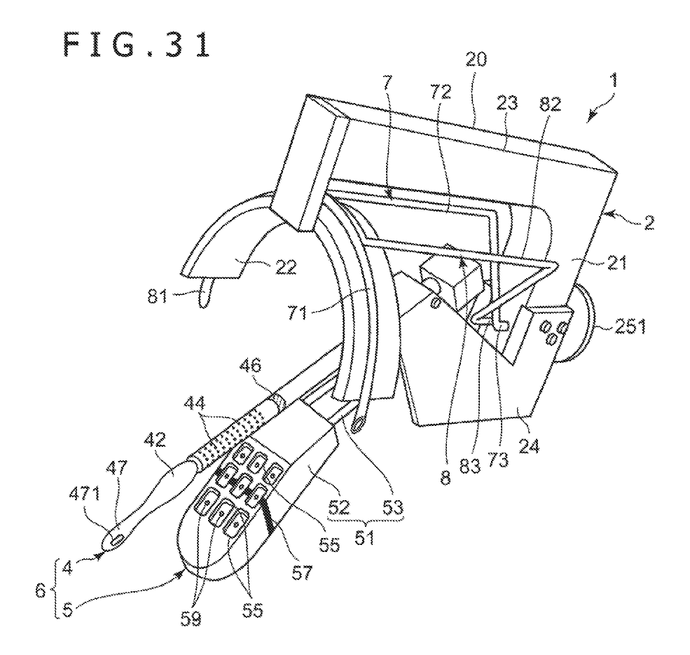

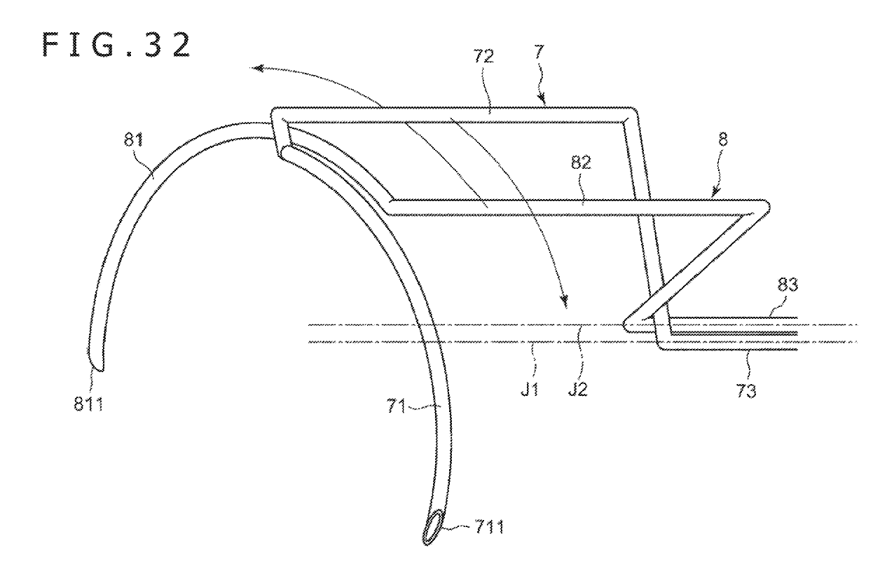

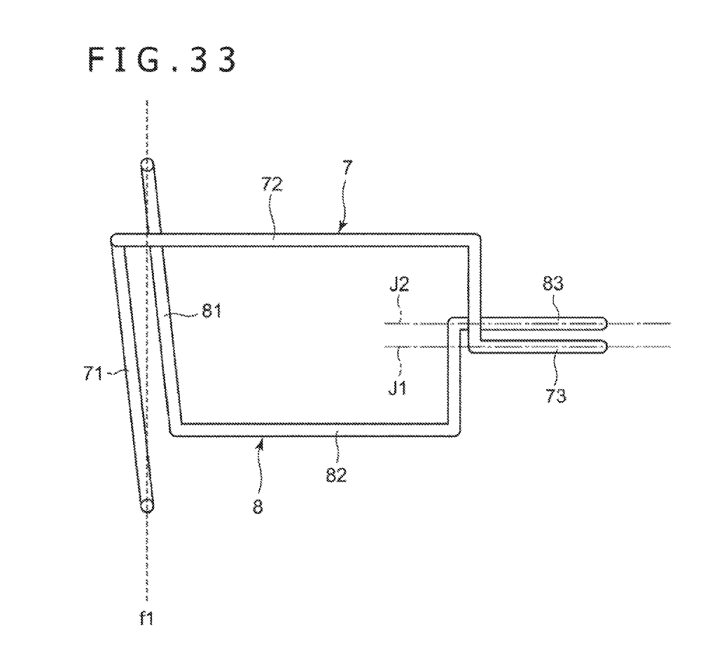

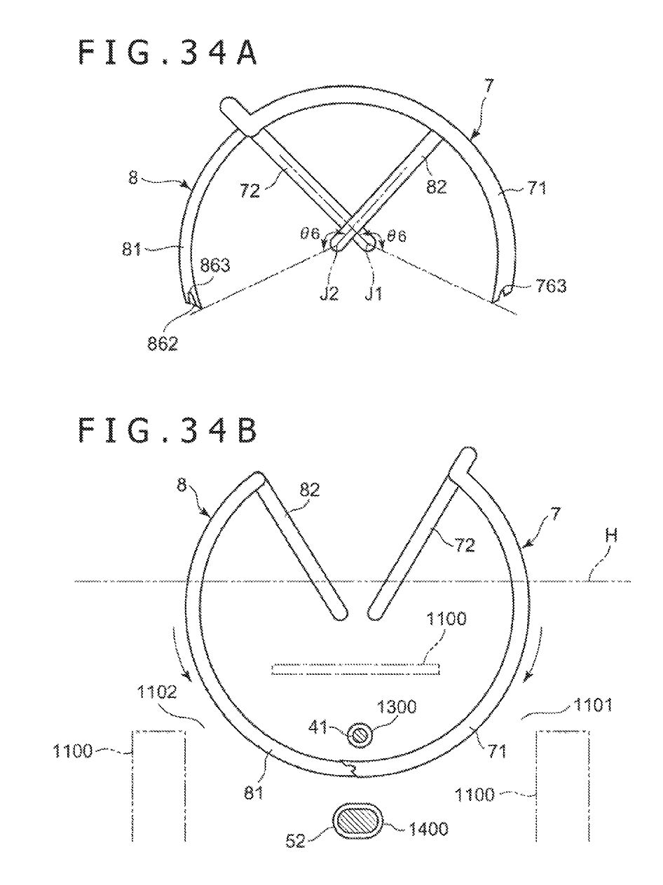

FIG. 31 is a perspective view illustrating a puncture apparatus according to a sixth embodiment of the present disclosure; FIG. 32 is a perspective view depicting puncture members possessed by the puncture apparatus of FIG. 31; FIG. 33 is a top plan view of the puncture members depicted in FIG. 32; and FIGS. 34A and 34B depict plan views for explaining rotary movement of the puncture members of FIG. 32, wherein FIG. 34A depicts an initial state, and FIG. 34B depicts a puncturing state.

Referring to these drawings, the sixth embodiment of the puncture apparatus will be described below. The following description will focus on differences of this sixth embodiment from the aforementioned embodiments, and the description of the same items as above will be omitted.

The sixth embodiment is the same as the aforementioned fourth embodiment except for a difference in the configuration of first and second puncture needles.

FIGS. 31, 32, and 33 illustrate an initial state of a puncture apparatus 1 according to this sixth embodiment. As depicted in these figures, in the puncture apparatus 1 in this embodiment, in the initial state, a proximal portion 79 of a puncture needle 71 and a proximal portion 89 of a puncture needle 81 are provided in an overlapping manner in the direction of axes J1 and J2 (the direction of a normal to a plane f1). In addition, the puncture needles 71 and 81 are each so disposed as not to interfere with the rotary movement of the opponent.

When the puncture needles 71 and 81 are rotationally moved toward their distal sides, starting from the initial state illustrated in FIG. 34A, a distal portion of the puncture needle 81 is inserted into a distal portion of the puncture needle 71, and the puncture needles 71 and 81 engage each other, as depicted in FIG. 34B. Note that in the puncture apparatus 1 in this embodiment, it is necessary to make puncture members 7 and 8 cross each other during such rotary movements; therefore, the puncture members 7 and 8 are so designed that the puncture member 8 is moved in the manner of passing through the inner side of the puncture member 7.

With the proximal portions 79 and 89 of the puncture needles 71 and 81 thus disposed in an overlapping manner in the direction of the axes J1 and J2, center angles θ6 of the puncture needles 71 and 81 can be set larger while suppressing the amounts of protrusion of the puncture needles 71 and 81 in the initial state. Therefore, the puncture needles 71 and 81 can be made to puncture a living body to an enlarged depth. Consequently, the puncture apparatus 1 can be sufficiently applied also to, for example, a relatively large patient having a large skinfold thickness, as depicted in FIG. 34B.

In addition, in this embodiment, the proximal portion 79 of the puncture needle 71 is located forwardly of the proximal portion 89 of the puncture needle 81, which helps enable the puncture needles 71 and 81 to be each so disposed as not to interfere with the rotary movement of the opponent.

In addition, as depicted in FIG. 33, the puncture needle 71 is formed in a spiral shape directed rearward along the axis J1; on the other hand, the puncture needle 81 is formed in a spiral shape directed forward along the axis J2. The puncture needle 81 is provided substantially in parallel to the puncture needle 71. In addition, a distal end of the puncture needle 71 and a distal end of the puncture needle 81 are both located in the plane f1, and are rotationally moved in the plane f1. With both the distal ends of the puncture needles 71 and 81 thus moved in the plane f1, puncturing by the puncture needles 71 and 81 can be performed relatively smoothly, and the distal ends of the puncture needles 71 and 81 can be made to reliably engage each other. Particularly, with the puncture needles 71 and 81 shaped spirally, the puncturing by the puncture needles 71 and 81 can be carried out with reduced resistance.

Note that the center angles θ6 of the puncture needles 71 and 81 are not particularly limited; for example, the center angles θ6 are preferably about 90 degrees to 170 degrees, more preferably about 120 degrees to 150 degrees. As a result, the puncture needles 71 and 81 can puncture a living body to an enlarged depth, as above-mentioned. In addition, the puncture needles 71 and 81 can be prevented from becoming excessively long, and can be made less susceptible to deformation (torsional deformation, flexural deformation). Consequently, accurate puncturing can be performed with the puncture needles 71 and 81.

According to this sixth embodiment, also, equivalent or similar effects to those of the aforementioned first embodiment can be produced.

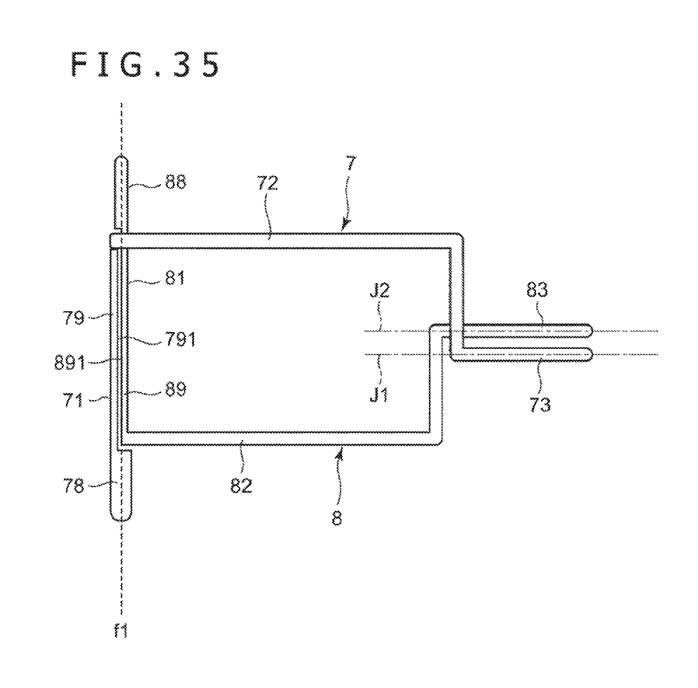

FIG. 35 is a top plan view illustrating puncture members possessed by a puncture apparatus according to a seventh embodiment of the present disclosure.

Referring to this drawing, the seventh embodiment of the puncture apparatus will be described below. The following description will focus on differences of the seventh embodiment from the aforementioned embodiments, and the description of the same items as above will be omitted.

This seventh embodiment is the same as the aforementioned sixth embodiment except for a difference in the configuration of first and second puncture needles.

As illustrated in FIG. 35, in a puncture apparatus 1 according to the seventh embodiment, in an initial state, a proximal portion 79 of a puncture needle 71 and a proximal portion 89 of a puncture needle 81 are provided in an overlapping manner in the extending direction of axes J1 and J2 (the direction of a normal to a plane f1). In addition, the proximal portion 79 of the puncture needle 71 is formed at its rear surface with a hollowed portion (recess) 791 recessed relative to a distal portion 78. The proximal portion 89 of the puncture needle 81 is formed at its front surface with a hollowed portion (recess) 891 recessed relative to a distal portion 88. In the initial state, the proximal portion 89 of the puncture needle 81 is located in the hollowed portion 791 of the puncture needle 71, whereas the proximal portion 79 of the puncture needle 71 is located in the hollowed portion 891 of the puncture needle 81. In accordance with an exemplary embodiment, the hollowed portion 791 functions as a relief portion for preventing interference with the puncture needle 81, whereas the hollowed portion 891 functions as a relief portion for preventing interference with the puncture needle 71. This configuration enables the puncture needles 71 and 81 to be disposed in an overlapping manner while adopting a simple configuration. In addition, the puncture needles 71 and 81 can entirely be located in the plane f1, and can be rotationally moved in the plane f1. Therefore, puncturing by the puncture needle 71 and 81 can be performed relatively smoothly.

According to this seventh embodiment, also, equivalent or similar effects to those of the aforementioned first embodiment can be produced.

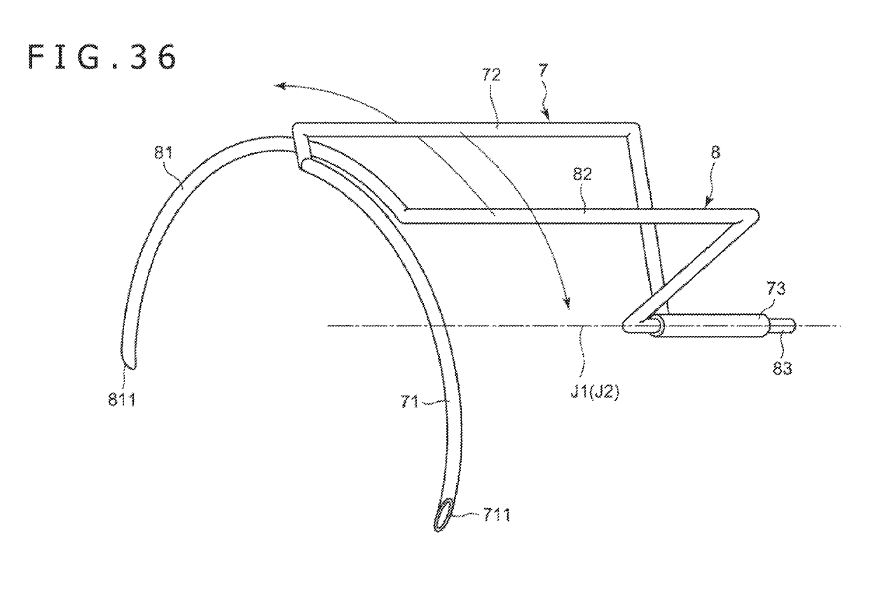

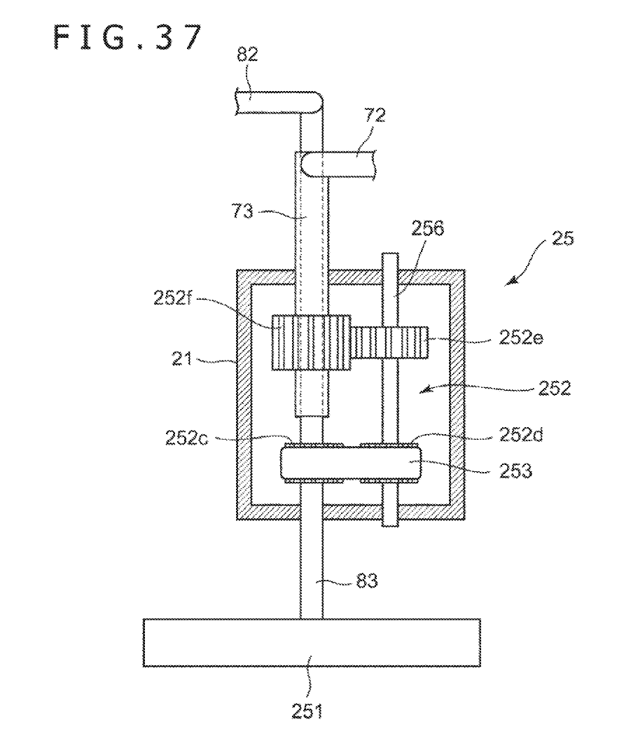

FIG. 36 is a perspective view illustrating puncture members possessed by a puncture apparatus according to an eighth embodiment of the present disclosure. FIG. 37 is a sectional view depicting a rotational operation unit possessed by the puncture apparatus according to the eighth embodiment.

Referring to these drawings, the eighth embodiment of the puncture apparatus will be described below. The following description will focus on differences of this eighth embodiment from the aforementioned embodiments, and the description of the same items as above will be omitted.

The eighth embodiment is the same as the aforementioned sixth embodiment except for a difference in the configuration of first and second puncture needles.

As depicted in FIG. 36, in a puncture apparatus 1 according to the eighth embodiment, a shaft portion 73 of a puncture member 7 is tube shaped, and a shaft portion 83 of a puncture member 8 is inserted inside the shaft portion 73. Therefore, a rotational axis J1 of the puncture member 7 and a rotational axis J2 of the puncture member 8 coincide with each other, which help enable the puncture members 7 and 8 to be rotationally moved in a coaxial manner, so that puncturing of a living body by puncture needles 71 and 81 can be performed relatively smoothly. In addition, distal portions of the puncture needles 71 and 81 can be made to reliably engage each other.

In accordance with an exemplary embodiment, as depicted in FIG. 37, a rotational operation unit 25 in this embodiment can include a handle 251 provided on the rear side of a bearing portion 21. The shaft portion 83 of the puncture member 8 is fixed to the handle 251. In addition, the rotational operation unit 25 has a conversion unit 252 incorporated in the bearing portion 21. The conversion unit 252 converts rotation of the handle 251 into rotary movement of the puncture needle 71 and rotary movement of the puncture needle 81. The conversion unit 252 can include four gears 252c, 252d, 252e and 252f and a belt 253. The gear 252c is fixed to the shaft portion 83, whereas the gear 252d is fixed to a shaft 256 provided in parallel to the shaft portion 83. The gears 252c and 252d are interlocked by the belt 253. The gear 252e is fixed to the shaft 256, whereas the gear 252f is fixed to the shaft portion 73 in the state of meshing with the gear 252e. The gear ratios of these four gears 252c to 252f are so set that the shaft portions 73 and 83 are rotated by equal rotational angles.

According to such a configuration, when the handle 251 is rotated clockwise, the puncture needle 81 is rotationally moved clockwise, and the puncture needle 71 is rotationally moved counterclockwise by the same angle as the puncture needle 81. In contrast, when the handle 251 is rotated counterclockwise, the puncture needle 81 is rotationally moved counterclockwise, and the puncture needle 71 is rotationally moved clockwise by the same angle as the puncture needle 81. According to the rotational operation unit 25 configured in this way, by rotating only the handle 251, a living body can be punctured by the puncture needles 71 and 81 simultaneously, such that the puncture apparatus 1 can be used relatively easily. Particularly, since the puncture needles 71 and 81 are rotationally moved by the same rotational angle, it can be easy to judge (expect) movements of the puncture needles 71 and 81, so that the puncture apparatus 1 can be used with enhanced safety.

According to this eighth embodiment, also, equivalent or similar effects to those of the aforementioned first embodiment can be produced.

Note that in place of this embodiment, another configuration may be adopted in which pulleys are used in place of the gears 252c and 252d, and a flat belt is used as the belt 253, which can help enable the shaft portion 83 (handle 251) to run idle in relation to the shaft portion 73. This ensures that, for example, when an excessive load (a load in excess of a predetermined value) is received due to an operation of the handle 251 in a condition where the puncture needle 71 is in abutment on a pelvis and further puncturing is impossible, the handle 251 runs idle in relation to the shaft portion 73, whereby further puncturing by the puncture needle 71 can be prevented from occurring. Consequently, mis-puncturing can be prevented, and the puncture apparatus 1 can be used with enhanced safety.

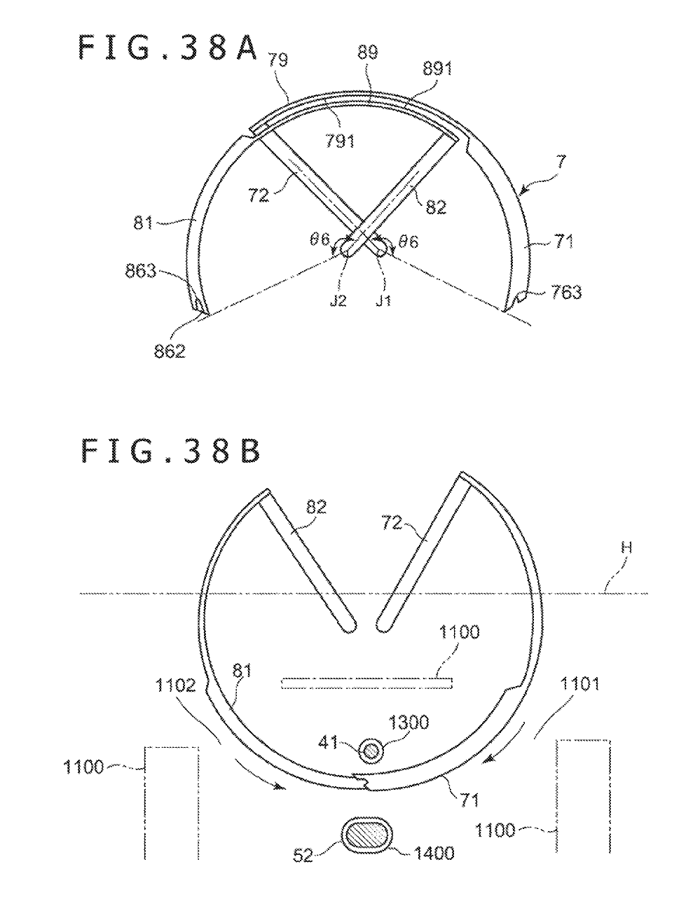

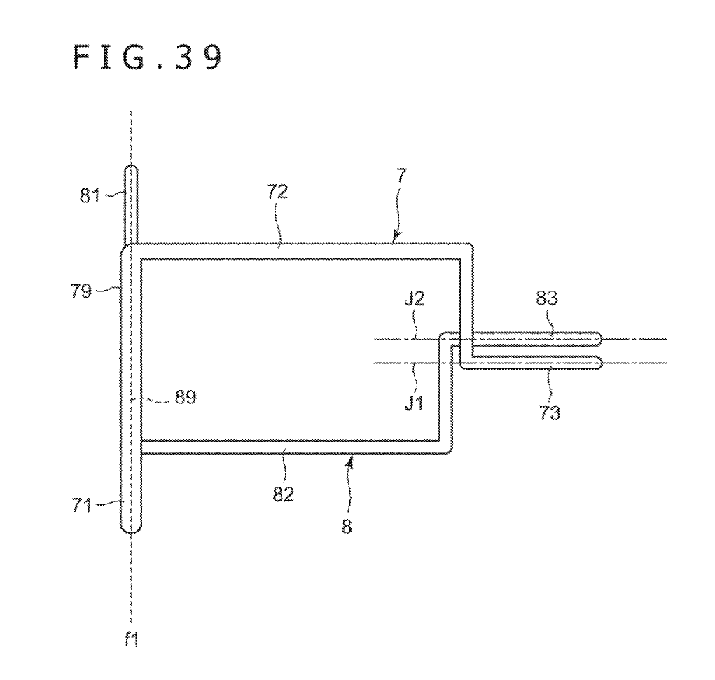

FIGS. 38A and 38B depict plan views illustrating puncture members possessed by a puncture apparatus according to a ninth embodiment of the present disclosure, wherein FIG. 38A depicts an initial state, and FIG. 38B depicts a puncturing state. FIG. 39 is a top plan view of the puncture members depicted in FIGS. 38A and 38B.

Referring to these drawings, the ninth embodiment of the puncture apparatus will be described below. The following description will focus on differences of this ninth embodiment from the aforementioned embodiments, and the description of the same items as above will be omitted.

The ninth embodiment is the same as the aforementioned sixth embodiment, except for a difference in the configuration of first and second puncture needles.

As illustrated in FIG. 38A, in a puncture apparatus 1 in this embodiment, in an initial state, a proximal portion 79 of a puncture needle 71 and a proximal portion 89 of a puncture needle 81 are provided in an overlapping manner in a direction orthogonal to axes J1 and J2 (in a direction in a plane f1). When the puncture needles 71 and 81 are rotationally moved toward their distal sides, starting from the initial state depicted in FIG. 38A, a distal portion of the puncture needle 81 is inserted into a distal portion of the puncture needle 71, as depicted in FIG. 38B. With the proximal portions 79 and 89 thus disposed in an overlapping manner, center angles of the puncture needles 71 and 81 can be set larger while suppressing the amounts of protrusion of the puncture needles 71 and 81 in the initial state. Therefore, the puncture needles 71 and 81 can be made to puncture a living body to an enlarged depth. Accordingly, the puncture apparatus 1 can be applied also to, for example, a relatively large patient having a large skinfold thickness.

In the puncture apparatus 1, the proximal portion 79 of the puncture needle 71 is formed on its inner circumferential side with a hollowed portion (recess) 791 recessed relative to a distal portion. The proximal portion 89 of the puncture needle 81 is formed on its outer circumferential side with a hollowed portion (recess) 891 recessed relative to a distal portion. In the initial state, the proximal portion 89 of the puncture needle 81 is located in the hollowed portion 791 of the puncture needle 71, whereas the proximal portion 79 of the puncture needle 71 is located in the hollowed portion 891 of the puncture needle 81. In accordance with an exemplary embodiment, the hollowed portion 791 functions as a relief portion for preventing interference with the puncture needle 81, whereas the hollowed portion 891 functions as a relief portion for preventing interference with the puncture needle 71. According to such a configuration, the puncture needles 71 and 81 can be disposed in an overlapping state while adopting a simple configuration.

In accordance with an exemplary embodiment, as depicted in FIG. 39, the puncture needles 71 and 81 are both located in the plane f1, and are rotationally moved in the plane f1. Therefore, puncturing by the puncture needles 71 and 81 can be performed with enhanced smoothness, and distal portions of the puncture needles 71 and 81 can be engaged with each other with higher reliability.

Note that the proximal portion 79 is located on the outside of the proximal portion 89 in this embodiment, the proximal portion 89 may, in contrast, be located on the outside of the proximal portion 79.

According to this ninth embodiment, also, equivalent or similar effects to those of the aforementioned first embodiment can be produced.

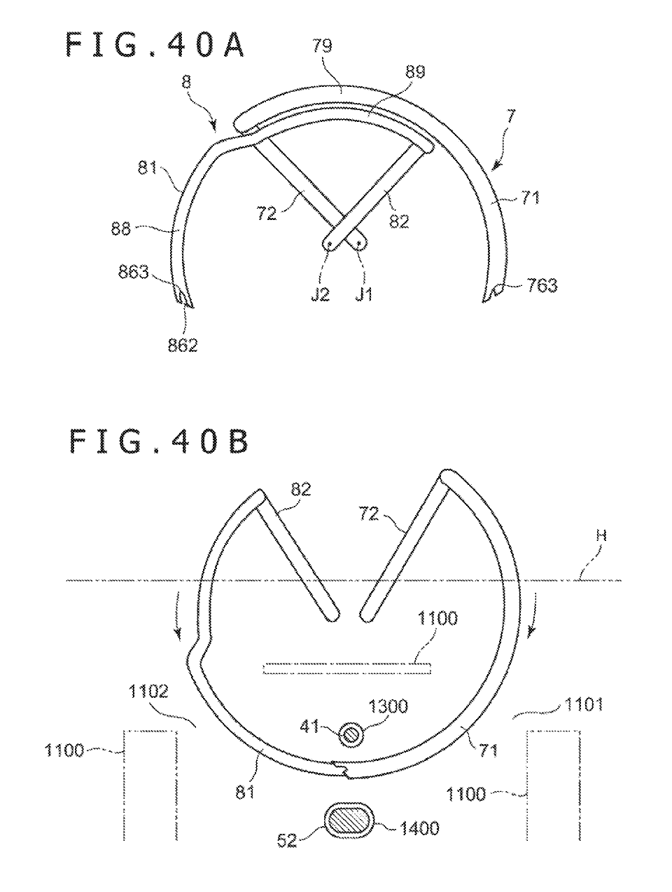

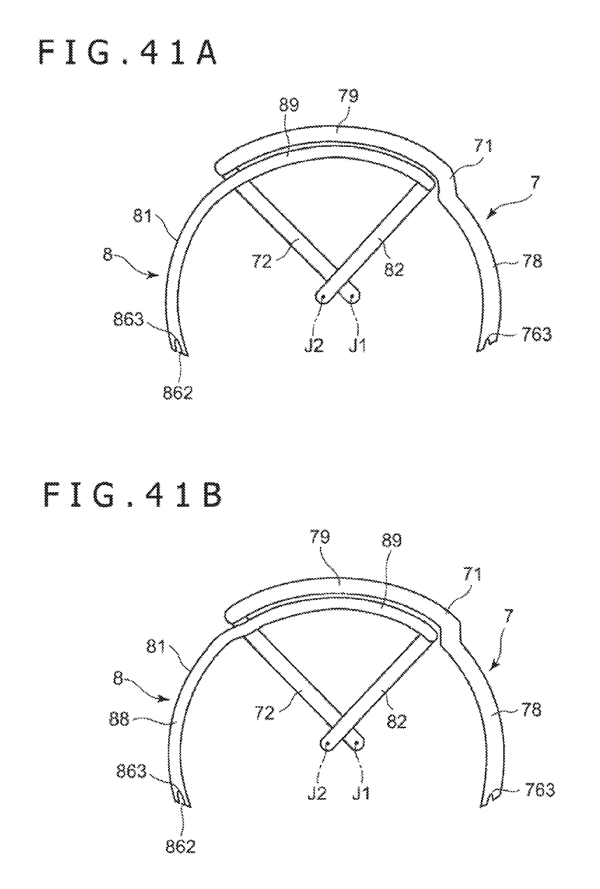

FIGS. 40A and 40B depict plan views illustrating puncture members possessed by a puncture apparatus according to a tenth embodiment of the present disclosure, wherein FIG. 40A depicts an initial state, and FIG. 40B depicts a puncturing state. FIGS. 41A and 41B are plan views illustrating a modification of the puncture members of FIGS. 40A and 40B.

Referring to these drawings, the tenth embodiment of the puncture apparatus will be described below. The following description will focus on differences of the tenth embodiment from the aforementioned embodiments, and the description of the same items as above will be omitted.

This tenth embodiment is the same as the aforementioned ninth embodiment except for a difference in the configuration of first and second puncture needles.

As illustrated in FIG. 40A, in a puncture apparatus 1 in this embodiment, in the initial state, a proximal portion 79 of a puncture needle 71 and a proximal portion 89 of a puncture needle 81 are provided in an overlapping manner in a direction orthogonal to axes J1 and J2 (in a direction in a plane f1). When the puncture needles 71 and 81 are rotationally moved toward their distal sides, starting from the initial state depicted in FIG. 40A, a distal portion of the puncture needle 81 is inserted into a distal portion of the puncture needle 71, as depicted in FIG. 40B. With the proximal portions 79 and 89 thus disposed in an overlapping manner, center angles of the puncture needles 71 and 81 can be set larger while suppressing the amounts of protrusion of the puncture needles 71 and 81 in the initial state. Therefore, the puncture needles 71 and 81 can be made to puncture a living body to an enlarged depth. Accordingly, the puncture apparatus 1 can be applied also to, for example, a relatively large patient having a large skinfold thickness.

In addition, the puncture needles 71 and 81 are both disposed in the plane f1, and are rotationally moved in the plane f1.

In accordance with an exemplary embodiment, the puncture needle 71 is arcuate in shape, and has a substantially constant radius of curvature over the whole region in the extending direction thereof. On the other hand, the puncture needle 81 has different radii of curvature at the proximal portion 89 and at a distal portion 88 of the puncture needle 81, although it is curved in an arcuate shape as a whole. Specifically, the puncture needle 81 can include the distal portion 88, which is arcuate in shape and has a radius of curvature approximately equal to that of the puncture needle 71, and the proximal portion 89 which is arcuate in shape and has a radius of curvature smaller than that of the puncture needle 71. The distal portion 88 and the proximal portion 89 are connected by a connection portion, which is gently curved (inclined). In the initial state, the proximal portion 89 of the puncture needle 81 is located on the inside of the proximal portion 79 of the puncture needle 71. In accordance with an exemplary embodiment, a space formed by positional deviation of the proximal portion 89 to the inside in relation to the distal portion 88 functions as a relief portion for preventing interference between the puncture needles 71 and 81. According to such a configuration, the puncture needles 71 and 81 can be disposed in an overlapping manner while adopting a simple configuration.

According to this tenth embodiment, also, equivalent or similar effects to those of the aforementioned first embodiment can be produced.

Note that configurations as depicted in FIGS. 41A and 41B may be adopted as modifications of the tenth embodiment.

In a modification as illustrated in FIG. 41A, a puncture needle 81 is arcuate in shape, and has a substantially constant radius of curvature over the entire region in the extending direction thereof. On the other hand, a puncture needle 71 can include a distal portion 78, which is arcuate in shape and has a radius of curvature approximately equal to that of the puncture needle 81, and a proximal portion 79 which is arcuate in shape and has a radius of curvature greater than that of the puncture needle 81. The distal portion 78 and the proximal portion 79 are connected by a connection portion, which is gently curved (inclined). In the initial state, the proximal portion 79 of the puncture needle 71 is located on the outside of the proximal portion 89 of a puncture needle 81.

In a modification as illustrated in FIG. 41B, a puncture needle 71 can include a distal portion 78 which is arcuate in shape and has a predetermined radius of curvature, and a proximal portion 79 which is arcuate in shape and has a radius of curvature greater than that of the distal portion 78. The distal portion 78 and the proximal portion 79 are connected by a connection portion, which is gently curved (inclined). On the other hand, a puncture needle 81 can include a distal portion 88 which is arcuate in shape and has a radius of curvature approximately equal to that of the distal portion 78 of the puncture needle 71, and a proximal portion 89 which is arcuate in shape and has a radius of curvature smaller than that of the distal portion 88. The distal portion 88 and the proximal portion 89 are connected by a connection portion which is gently curved (inclined). In addition, the proximal portion 89 has a radius of curvature smaller than that of the proximal portion 79, and the difference between the distal portion 78 and the proximal portion 79 in radius of curvature is approximately equal to the difference between the distal portion 88 and the proximal portion 89 in radius of curvature. In the initial state, the proximal portion 79 of the puncture needle 71 is located on the outside of the proximal portion 89 of the puncture needle 81.

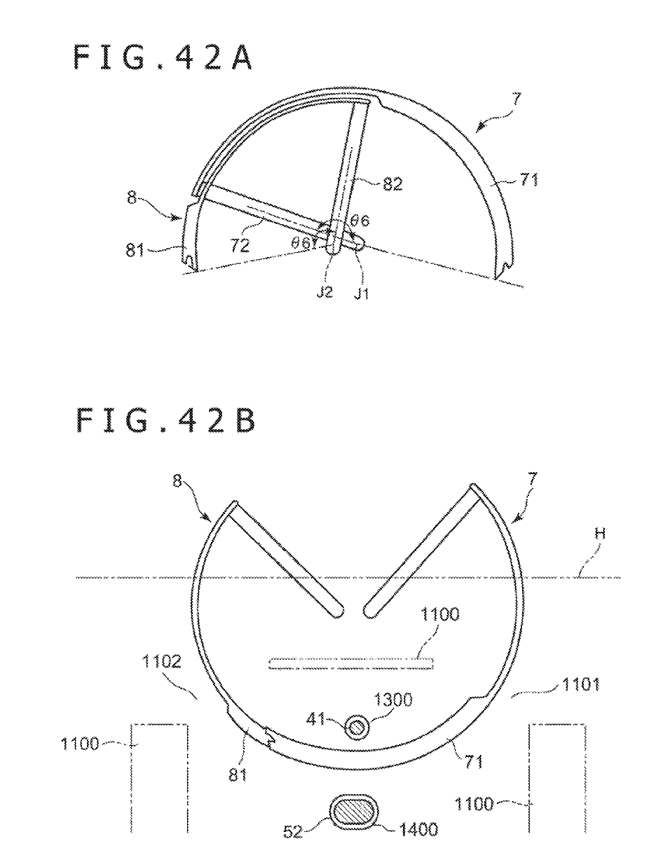

FIGS. 42A and 42B depict plan views illustrating puncture members possessed by a puncture apparatus according to an eleventh embodiment of the present disclosure, wherein FIG. 42A depicts an initial state, and FIG. 42B depicts a puncturing state.

Referring to these drawings, the eleventh embodiment of the puncture apparatus will be described below. The following description will focus on differences of the eleventh embodiment from the aforementioned embodiments, and the description of the same items as above will be omitted.

This eleventh embodiment is the same as the aforementioned ninth embodiment except for a difference in the configuration of first and second puncture needles.

As illustrated in FIG. 42A, in a puncture apparatus 1 according to the eleventh embodiment, a puncture needle 71 is designed to be greater than a puncture needle 81 in center angle θ6. In addition, a rotational operation unit 25 is so configured that as a handle 251 is rotated, the puncture needle 71 is rotationally moved with a rotational angle greater than that of the puncture needle 81. Note that the rotational angles of the puncture needles 71 and 81 can be appropriately changed by regulating the gear ratio of gears 252a and 252b.

Therefore, when the puncture needles 71 and 81 are rotationally moved toward their distal sides, starting from the initial state depicted in FIG. 42A, the puncture needles 71 and 81 engage each other at a position deviated from a position just between a urethra 1300 and a vagina 1400 as depicted in FIG. 42B. With the puncture needles 71 and 81 thus made to engage each other at a position spaced away from the urethra 1300 and the vagina 1400, puncturing the urethra 1300 or the vagina 1400 by mistake can be effectively prevented from occurring, so that the procedure can be carried out with enhanced safety.

Particularly, in this embodiment, the puncture needle 71 which is thicker (diametrically larger) and stiffer than the puncture needle 81 is set longer, whereby the puncture needle 81 which is originally low in rigidity (stiffness) is prevented from being further lowered in rigidity. Therefore, the rigidity of the puncture needles 71 and 81 can be maintained sufficiently, so that puncturing by the puncture needles 71 and 81 can be performed with enhanced accuracy. Note that, in contrast to this embodiment, the puncture needle 81 may be set longer, and the puncture needle 71 shorter. In this case, since the puncture needle 81 which is thinner (diametrically smaller) than the puncture needle 71 is set longer, the procedure will be accordingly lower in invasiveness.

Note that the center angle θ6 of the puncture needle 71 is not particularly limited, so long as the puncture needle 71 can pass through a region between the urethra 1300 and the vagina 1400. The center angle θ6 is preferably, for example, about 120 degrees to 200 degrees, more preferably about 140 degrees to 170 degrees. On the other hand, the center angle θ6 of the puncture needle 81 is not specifically restricted, and is preferably, for example, about 50 degrees to 100 degrees, more preferably about 60 degrees to 80 degrees.

According to this eleventh embodiment, also, equivalent or similar effects as those of the aforementioned first embodiment can be produced.

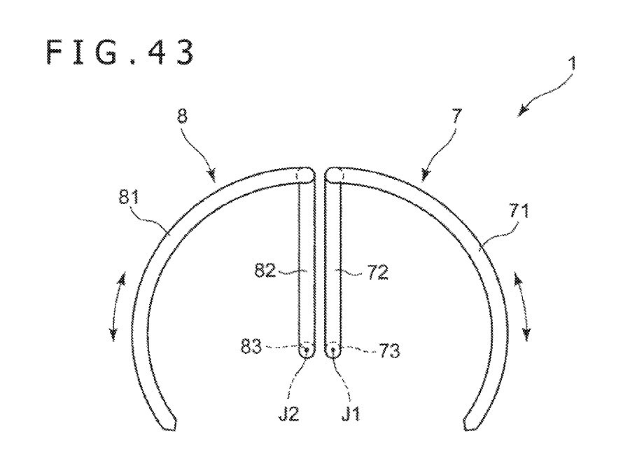

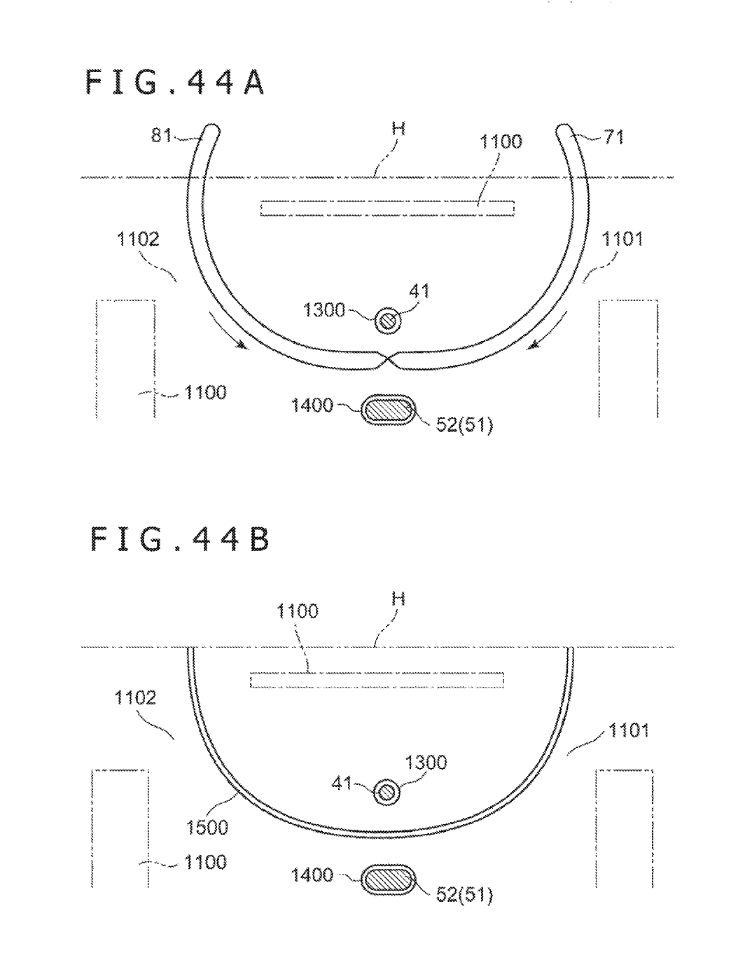

FIG. 43 is a plan view illustrating puncture members possessed by a puncture apparatus according to a twelfth embodiment of the present disclosure. FIGS. 44A and 44B are views for explaining an operating procedure of the puncture apparatus having the puncture members of FIG. 43.

Referring to these drawings, the twelfth embodiment of the puncture apparatus will be described below. The following description will focus on differences of the twelfth embodiment from the aforementioned embodiments, and the description of the same items as above will be omitted.

This twelfth embodiment is the same as the aforementioned fourth embodiment except for a difference in the configuration of first and second puncture needles.

As illustrated in FIG. 43, in a puncture apparatus 1 in this embodiment, a puncture needle 71 is not provided with an implant retaining portion 76, and a puncture needle 81 is not provided with an implant receiving portion 86. Therefore, in the puncture apparatus 1 in this embodiment, transfer of an implant 9 from the puncture needle 71 to the puncture needle 81 is impossible. In accordance with an exemplary embodiment, the puncture needles 71 and 81 have only the inherent function of puncturing a living body. Since the implant retaining portion 76 and the implant receiving portion 86 are thus omitted, the puncture needles 71 and 81 are solid and do not have through-holes 713 and 813 therein.

Now, a method of embedding the implant 9 into a living body by use of the puncture apparatus 1 according to this embodiment will be described below.

First, through the same step as in the aforementioned first embodiment, the puncture needles 71 and 81 are made to puncture a living body, and distal ends of the puncture needles 71 and 81 are abutted on each other in the living body, as depicted in FIG. 44A. Next, the puncture needles 71 and 81 are rotationally moved toward their proximal sides and are thereby drawn out, as depicted in FIG. 44B. As a result, a puncture hole 1500 in which to embed the implant 9 is formed in the living body. Thereafter, the implant 9 is disposed into the puncture hole 1500, whereby the implant 9 can be embedded in the living body. Note that the method for disposing the implant 9 into the puncture hole 1500 is not specifically restricted. For example, the following method may be used. First, a guide wire is introduced into the puncture hole 1500 so that both end portions of the guide wire are exposed to the exterior. Then, the implant 9 is connected to one end of the guide wire, and the other end of the guide wire is pulled, whereby the implant 9 can be disposed in the puncture hole 1500.

According to this twelfth embodiment, also, equivalent or similar effects to those of the aforementioned first embodiment can be produced.

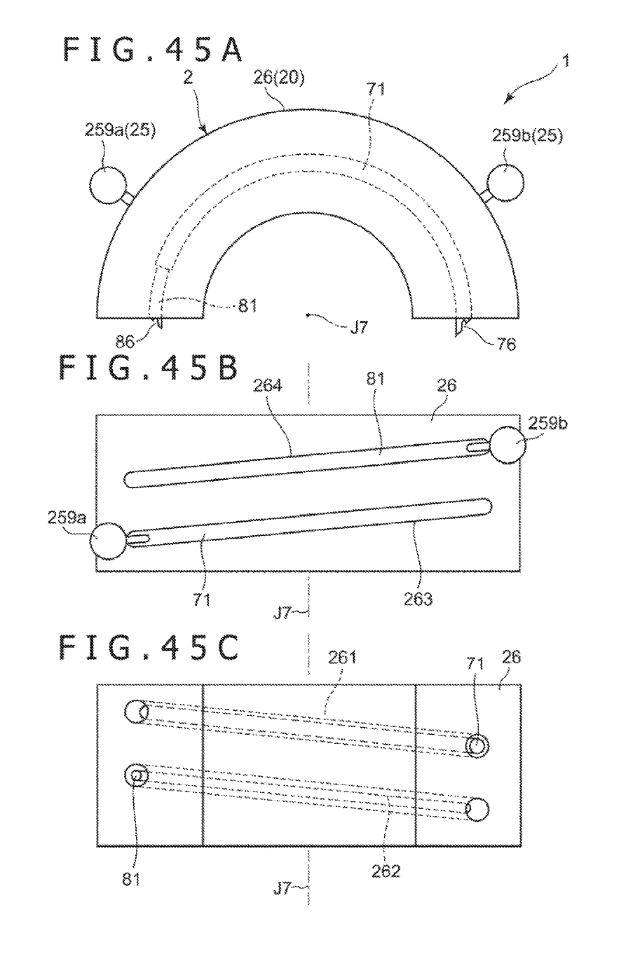

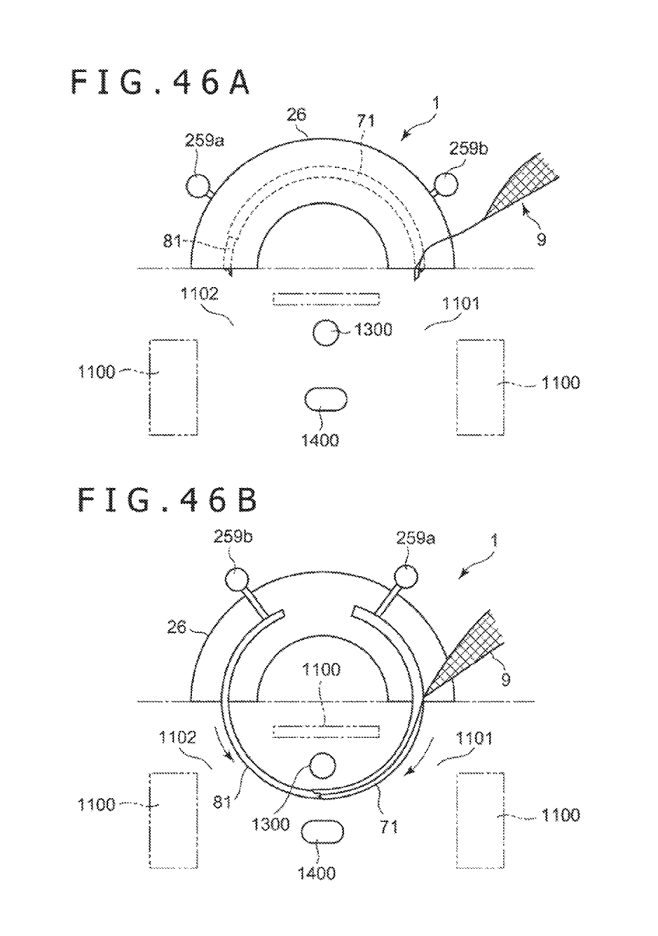

FIGS. 45A to 45C depict views illustrating an initial state of a puncture apparatus according to a thirteenth embodiment of the present disclosure, wherein FIG. 45A is a front view, FIG. 45B is a top plan view, and FIG. 45C is a bottom view. FIGS. 46A and 46B are views for explaining an operating procedure of the puncture apparatus depicted in FIGS. 45A to 45C.

Referring to these drawings, the thirteenth embodiment of the puncture apparatus will be described below. The following description will focus on differences of the thirteenth embodiment from the aforementioned embodiments, and the description of the same items as above will be omitted.

As illustrated in FIG. 45A, a puncture apparatus 1 in this embodiment can include a frame 2 having a frame main body 20 and a rotational operation unit 25, and puncture needles 71 and 81. The frame main body 20 can include a support portion 26, which supports and accommodates the puncture needles 71 and 81 in a rotationally movable manner. The support portion 26 is substantially U-shaped in form along the shape of the puncture needles 71 and 81, and its bottom surfaces constitute contact surfaces brought into contact with a living body tissue when the puncture apparatus 1 is used. The rotational operation unit 25 can include a knob 259a interlocked to a proximal portion of the puncture needle 71, and a knob 259b interlocked to a proximal portion of the puncture needle 81. By manually operating the knobs 259a and 259b, the puncture needles 71 and 81 can be operated relatively easily.

As depicted in FIG. 45C, the support portion 26 is formed therein with a guiding hole (guiding portion) 261 for guiding the puncture needle 71 for rotary movement, and a guiding hole (guiding portion) 262 for guiding the puncture needle 81 for rotary movement. Both ends of the guiding hole 261 are opening in the bottom surfaces of the support portion 26. Through the openings, the puncture needle 71 can be protruded to outside of the support portion 26 and be retracted into the support portion 26. Similarly, both ends of the guiding hole 262 are opening in the bottom surfaces of the support portion 26. Through the openings, the puncture needle 81 can be protruded to outside of the support portion 26 and be retracted into the support portion 26. With such guiding holes 261 and 262 thus provided, the puncture needles 71 and 81 can be rotationally moved with enhanced smoothness.

In addition, as illustrated in FIG. 45B, the support portion 26 is formed in its outer circumferential surface with a slot 263 communicating with the guiding hole 261, and the knob 259a is protruding from the slot 263. Similarly, the support portion 26 is formed in its outer circumferential surface with a slot 264 communicating with the guiding hole 262, and the knob 259b is protruding from the slot 264. The slot 263 is a hole with an end, and abutment of the knob 259a against a distal portion of the slot 263 can prevent the puncture needle 71 from further rotary movement. Similarly, the slot 264 is a hole with an end, and abutment of the knob 259b against a distal portion of the slot 264 can help prevent the puncture needle 81 from further rotary movement. In accordance with an exemplary embodiment, the slots 263 and 264 function also as limiting portions for limiting the rotational ranges of the puncture needles 71 and 81 (particularly, the amounts of protrusion of the puncture needles 71 and 81). With such limiting portions provided, excessive protrusion of the puncture needles 71 and 81 can be prevented, and distal portions of the puncture needles 71 and 81 can be made to engage each other relatively safely.

The puncture needles 71 and 81 accommodated in the support portion 26 configured as above are both curved in a substantially spiral shape. The puncture needles 71 and 81 are supported by and accommodated in the support portion 26, with their distal ends oriented to opposite sides and in the state of both being rotationally movable about a rotational axis J7. Since the rotational axes of the puncture needles 71 and 81 coincide with each other, the puncture needles 71 and 81 can be rotationally moved in a coaxial manner. Accordingly, puncturing of a living body by the puncture needles 71 and 81 can be performed with enhanced smoothness. Note that the rotational axis J7 coincides with center axes of the puncture needles 71 and 81.

The puncture needles 71 and 81 have substantially the same center angle and a substantially the same radius of curvature. In addition, the puncture needles 71 and 81 are disposed substantially in parallel to each other in the manner of overlapping each other in the direction of the rotational axis J7 so that their proximal portions will not interfere with each other. With the puncture needles 71 and 81 thus provided in the overlapping manner in the direction of the rotational axis J7, the center angles of the puncture needles 71 and 81 can be set larger while suppressing the amounts of protrusion of the puncture needles 71 and 81 in the initial state. Therefore, the puncture needles 71 and 81 can be made to puncture a living body to an enlarged depth. Accordingly, the puncture apparatus 1 can be sufficiently applied to, for example, a relatively large patient having a large skinfold thickness.

Note that the center angles of the puncture needles 71 and 81 are not particularly limited, and are preferably, for example, about 90 degrees to 180 degrees, more preferably about 120 degrees to 150 degrees. With such center angles, the puncture needles 71 and 81 can puncture a living body sufficiently to the inside of the living body, and, further, the distal portions of the puncture needles 71 and 81 can be engaged with each other in the living body. In accordance with an exemplary embodiment, the puncture needles 71 and 81 can be prevented from becoming excessively long, and can be made less susceptible to deformation (torsional deformation, flexural deformation). Accordingly, accurate puncturing by the puncture needles 71 and 81 can be performed.

The distal ends of the puncture needle 71 and the puncture needle 81 are both moved along the same spiral trajectory, which helps enable smooth puncturing by the puncture needles 71 and 81, and helps enable the distal portions of the puncture needles 71 and 81 to engage each other with enhanced reliability. Particularly, by forming the puncture needles 71 and 81 in a substantially spiral shape, puncturing by the puncture needles 71 and 81 can be conducted with low resistance.

The puncture needle 71 is tube shaped, and is provided at its distal portion with an implant retaining portion 76 (a groove 763). On the other hand, the puncture needle 81 is tube shaped with a smaller thickness (diametric size) than the puncture needle 71, and its distal portion can be inserted into the puncture needle 71. The puncture needle 81 is provided at its distal portion with an implant receiving portion 86 (a groove 863 and a claw portion 862). Therefore, when the puncture needles 71 and 81 are rotationally moved toward their distal sides and their distal portions are thereby engaged with each other, an implant 9 can be transferred from the puncture needle 71 to the puncture needle 81.

Now, a method of embedding the implant 9 into a living body by use of the puncture apparatus 1 in this embodiment will be described.

First, as depicted in FIG. 46A, the puncture apparatus 1 is so positioned that the distal end of the puncture needle 71 is located on an obturator foramen 1101 and the distal end of the puncture needle 81 is located on an obturator foramen 1102. In the condition where the puncture apparatus 1 is thus positioned, the bottom surfaces of the support portion 26 are in abutment on the living body tissue. Next, the implant 9 is placed in the groove 763 of the puncture needle 71, after which the knobs 259a and 259b are operated to rotationally move the puncture needles 71 and 81 in opposite directions (different directions), as depicted in FIG. 46B, thereby making the puncture needles 71 and 81 puncture the living body and inserting the distal portion of the puncture needle 81 into the distal portion of the puncture needle 71. In this instance, the implant 9 is guided into the living body together with the puncture needle 71, starting from its distal end, and transferred onto the puncture needle 81. Note that the puncturing order of the puncture needles 71 and 81 is not particularly limited; for example, puncturing by the puncture needle 71 may be followed by puncturing by the puncture needle 81, or, in contrast, puncturing by the puncture needle 81 may be followed by puncturing by the puncture needle 71. Further, puncturing by the puncture needle 71 and puncturing by the puncture needle 81 may be performed simultaneously. Among the aforesaid three modes, the mode of puncturing by the puncture needle 71 first and then puncturing by the puncture needle 81 is preferred. Where puncturing by the puncture needle, which is thicker (larger in diametric size) is conducted first, it can be easier to make the puncture needles 71 and 81 engage each other in the living body.

Next, the knobs 259a and 259b are operated to draw the puncture needles 71 and 81 out of the living body. In this instance, the implant 9 is partly discharged to the exterior of the living body together with the puncture needle 81, starting from its distal end. As a result of these operations, an implant main body 91 is left embedded in the living body.

As has been described above, according to the puncture apparatus 1, placing the implant 9 indwelling can be coped with only a minimally invasive procedure such as puncturing by the puncture needles 71 and 81, without need to perform a highly invasive procedure such as incision. Therefore, the burden on the patient is relatively light, and safety for the patient is relatively high. Particularly, since a living body is punctured from both sides by use of the two puncture needles 71 and 81, the lengths of the puncture needles 71 and 81 can be set short as compared, for example, with a case of puncturing a living body from one side by use of a single puncture needle. Accordingly, the puncture needles 71 and 81 can be restrained from flexure or torsion due to resistance received at the time of puncturing. Consequently, puncturing by the puncture needles 71 and 81 can be performed with enhanced accuracy and safety.

According to this thirteenth embodiment, also, equivalent or similar effects to those of the aforementioned first embodiment can be produced.

Note that the distal ends of the puncture needles 71 and 81 are protruding from the support portion 26 in the initial state in this embodiment, this is not restrictive; in the initial state, the puncture needles 71 and 81 may be in the state of being retracted in the support portion 26. In addition, though not depicted, the aforesaid procedure is preferably conducted in a condition where an insertion device 6 is mounted to a living body, specifically, in a condition where a urethral-insertion portion 41 is inserted in a urethra 1300 and a vaginal-insertion portion 51 is inserted in a vagina 1400, which can help enable puncturing by the puncture needles 71 and 81 to be carried out with enhanced safety.

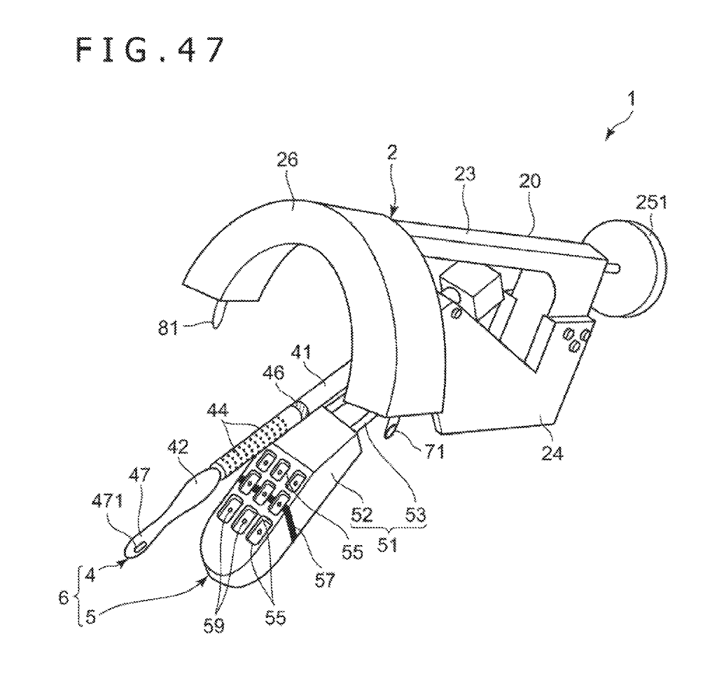

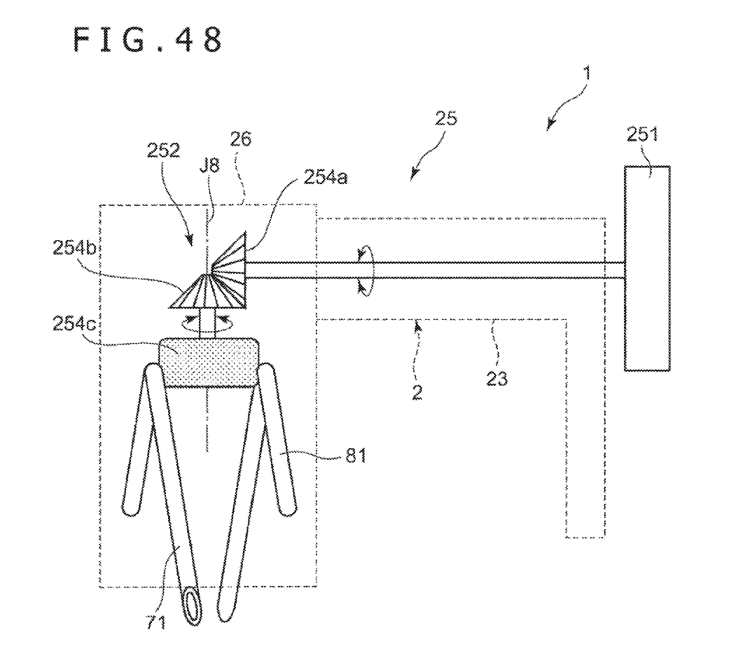

FIG. 47 is a perspective view illustrating a puncture apparatus according to a fourteenth embodiment of the present disclosure. FIG. 48 is a plan view depicting the configuration of a rotational operation unit possessed by the puncture apparatus of FIG. 47.

Referring to these drawings, the fourteenth embodiment of the puncture apparatus will be described below. The following description will focus on differences of the fourteenth embodiment from the aforementioned embodiments, and the description of the same items as above will be omitted.

This fourteenth embodiment is the same as the aforementioned thirteenth embodiment except for differences in the configuration of a frame and a rotational operation unit.

As illustrated in FIG. 47, in a puncture apparatus 1 in this embodiment, a frame main body 20 can include a support portion 26, a fixation portion 24 for fixing an insertion device 6, and an interlock portion 23 for interlocking the support portion 26 and the fixation portion 24. In addition, the insertion device 6 (a urethral-insertion device 4 and a vaginal-insertion device 5) is fixed to the frame main body 20.

The frame main body 20 can determine positional relationships of puncture needles 71 and 81, the urethral-insertion device 4 and the vaginal-insertion device 5 such that the puncture needles 71 and 81 are abutted on each other between the urethral-insertion device 4 and the vaginal-insertion device 5 without colliding against the urethral-insertion device 4 and the vaginal-insertion device 5. In accordance with an exemplary embodiment, for example, the frame main body 20 can restrict the positional relationships of the puncture needles 71 and 81, the urethral-insertion device 4 and the vaginal-insertion device 5 such that when the puncture needles 71 and 81 are rotationally moved toward their distal sides, the distal end of at least one of the puncture needles 71 and 81 (an extension line in the puncturing direction of the puncture needles 71 and 81) passes through a position on the farther side from a rotational axis 7 than the urethral-insertion device 4, and distal portions of the puncture needles 71 and 81 engage each other.

In addition, the frame main body 20 is provided with a rotational operation unit 25 for rotationally moving the puncture needles 71 and 81 in an interlocked manner, simultaneously, in opposite directions and by equal angles. The provision of such a rotational operation unit 25 enables the puncture apparatus 1 to be operated easily and enables a time taken for a procedure to be shortened, as compared, for example, with a case where the puncture needles 71 and 81 are operated manually as in the aforementioned thirteenth embodiment. In addition, movements of the puncture needles 71 and 81 can be controlled in a fixed manner, so that the puncture apparatus 1 can be used properly and relatively safely.

As illustrated in FIG. 48, the rotational operation unit 25 can include a handle 251 which is provided on the rear side of the interlock portion 23 and rotatably borne on the interlock portion 23, and a conversion unit 252 which converts rotation of the handle 251 into rotary movement of the puncture needle 71 and rotary movement of the puncture needle 81. In accordance with an exemplary embodiment, the conversion unit 252 can include a bevel gear 254a fixed to a shaft of the handle 251, a bevel gear 254b meshing with the bevel gear 254a, and a substantially cylindrical rotary body 254c fixed to a shaft of the bevel gear 254b. The rotation of the handle 251 is inclined by 90 degrees by the bevel gears 254a and 254b; therefore, when the handle 251 is rotated, the rotary body 254c is rotated about an axis J8.

The rotary body 254c is formed from a material having a comparatively high frictional resistance such as rubber material, is disposed between the puncture needles 71 and 81, and in contact with both the puncture needles 71 and 81. When the rotary body 254c is rotated by an operation of the handle 251, therefore, the puncture needles 71 and 81 are rotationally moved attendantly on the rotation of the rotary body 254c, in an interlocked manner, simultaneously, in opposite directions, and by substantially equal rotational angles. According to such a configuration using the gears, the rotational operation unit 25 can be realized with a simple configuration.

Particularly, in this embodiment, the rotary body 254c can run idle in relation to the puncture needles 71 and 81, which can help ensure that, for example, when an excessive load (a load in excess of a predetermined value) is received due to an operation of the handle 251 in a condition where any of the puncture needles 71 and 81 is in abutment on a pelvis and further puncturing is impossible, the rotary body 254c runs idle in relation to the puncture needles 71 and 81, whereby further puncturing by the puncture needles 71 and 81 can be prevented from occurring. Consequently, mis-puncturing can be prevented, and the puncture apparatus 1 can be used with enhanced safety.

According to this fourteenth embodiment, equivalent or similar effects to those of the aforementioned first embodiment can be produced.

Note that the frame main body 20 has the interlock portion 23 and the fixation portion 24 in this embodiment, these components may be omitted. In this case, for example, the handle 251 may be disposed on the back side or the upper side of the support portion 26. In accordance with an exemplary embodiment, the insertion device 6 may also be omitted.

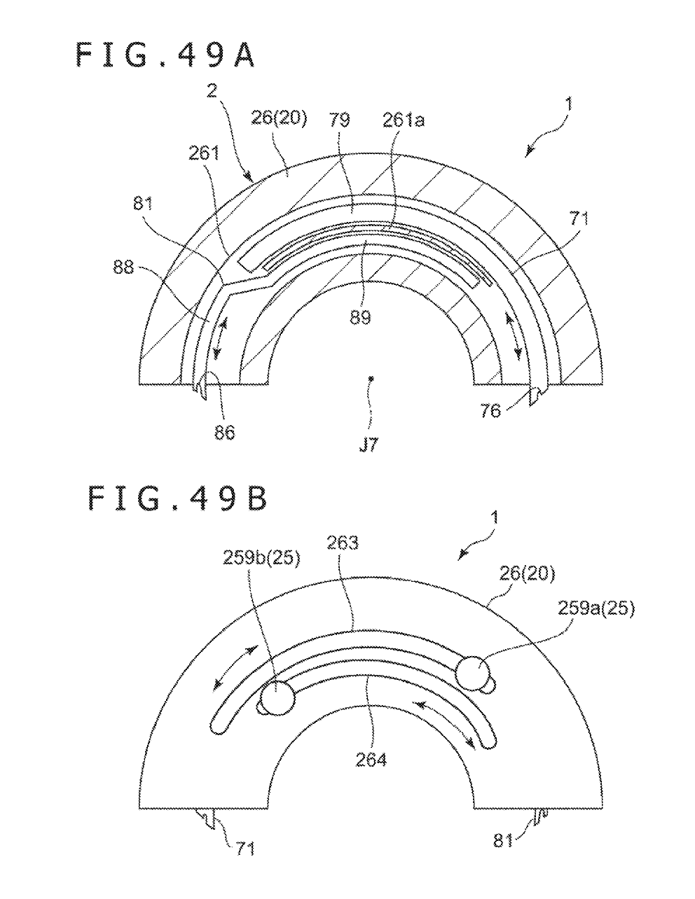

FIGS. 49A and 49B depict views illustrating an initial state of a puncture apparatus according to a fifteenth embodiment of the present disclosure, wherein FIG. 49A is a sectional view as viewed from the front side, and FIG. 49B is a back view.

Referring to these drawings, the fifteenth embodiment of the puncture apparatus will be described below. The following description will focus on differences of this fifteenth embodiment from the aforementioned embodiments, and the description of the same items as above will be omitted.

The fifteenth embodiment is the same as the aforementioned thirteenth embodiment except for a difference in the configuration of puncture needles.

As illustrated in FIGS. 49A and 49B, a puncture apparatus 1 in this embodiment can include a support portion 26, puncture needles 71 and 81, and knobs 259a and 259b. With the knobs 259a and 259b operated manually, the puncture needles 71 and 81 can be operated.

As depicted in FIGS. 49A and 49B, the puncture needles 71 and 81 are supported by and accommodated in the support portion 26 with their distal ends oriented in opposite directions and in the state of both being rotationally movable about a rotational axis J7. The puncture needles 71 and 81 have substantially equal center angles. The puncture needles 71 and 81 are provided in the same plane to which the rotational axis J7 is normal, and are rotationally moved in the plane. The puncture needles 71 and 81 are so disposed that their proximal portions 79 and 89 overlap each other in a direction in the plane (in a direction orthogonal to the rotational axis J7).

The puncture needle 71 is curved in an arcuate shape, and has a substantially constant radius of curvature over the whole region in the extending direction thereof. On the other hand, the puncture needle 81 can include a distal portion 88 which is curved in an arcuate shape and has a radius of curvature substantially equal to that of the puncture needle 71, and a proximal portion 89 which is curved in an arcuate shape and has a radius of curvature smaller than that of the puncture needle 71. The distal portion 88 and the proximal portion 89 are connected by a connection portion, which is gently curved (inclined). In an initial state, the proximal portion 89 of the puncture needle 81 is located inside a proximal portion 79 of the puncture needle 71.

The support portion 26 has a substantially U-shaped form along the shape of the puncture needles 71 and 81. The support portion 26 is provided therein with a guiding hole 261 for guiding the puncture needles 71 and 81 for rotary movement. On the inner side of the guiding hole 261, further, a screen 261a is provided for guiding the puncture needles 71 and 81 individually.

In addition, the support portion 26 is provided on a back side with slots 263 and 264 communicating with the guiding hole 261. The knob 259a is protruding from the slot 263, while the knob 259b is protruding from the slot 264. The slots 263 and 264 are each a hole with an end. With the knobs 259a and 259b in abutment on distal end portions of the slots 263 and 264, further rotary movement of the puncture needles 71 and 81 can be prevented.

According to this fifteenth embodiment, also, equivalent or similar effects to those of the aforementioned first embodiment can be produced.

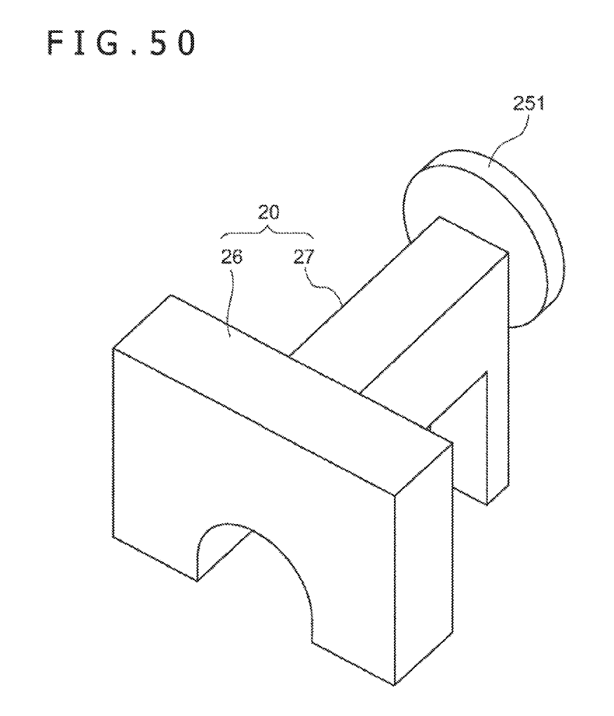

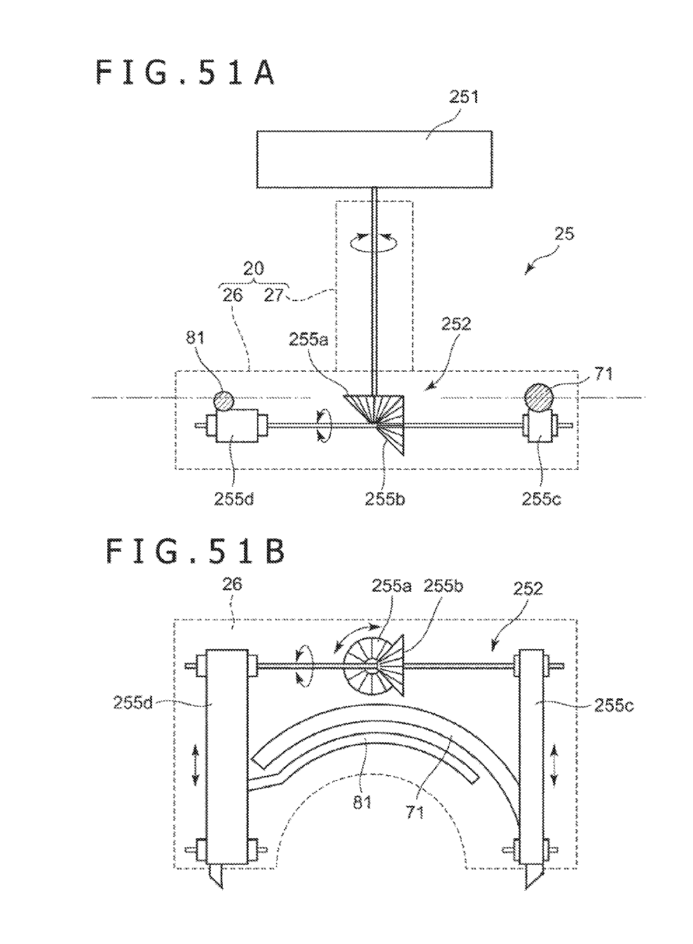

FIG. 50 is a perspective view illustrating a puncture apparatus according to a sixteenth embodiment of the present disclosure. FIGS. 51A and 51B depict views illustrating the configuration of a rotational operation unit possessed by the puncture apparatus of FIG. 50, wherein FIG. 51A is a top plan view, and FIG. 51B is a front view.

Referring to these drawings, the sixteenth embodiment of the puncture apparatus will be described below. The following description will focus on differences of the sixteenth embodiment from the aforementioned embodiments, and the description of the same items as above will be omitted.

This sixteenth embodiment is the same as the aforementioned fifteenth embodiment except for a difference in the configuration of a rotational operation unit.

As illustrated in FIG. 50, in a puncture apparatus 1 in this embodiment, a frame main body 20 can include a support portion 26, and a grasping portion 27 connected to the rear of an accommodating portion. The frame main body 20 is provided with a rotational operation unit 25 for rotationally moving puncture needles 71 and 81 in an interlocked manner, simultaneously, in opposite directions, and by substantially equal rotational angles. The provision of such a rotational operation unit 25 enables the puncture apparatus 1 to be operated easily and enables a time taken for a procedure to be shortened, as compared, for example, with a case of operating the puncture needles 71 and 81 manually as in the aforementioned fifteenth embodiment. In addition, movements of the puncture needles 71 and 81 can be controlled in a fixed manner, so that the puncture apparatus 1 can be used properly and safely.

As depicted in FIGS. 51A and 51B, the rotational operation unit 25 can include a handle 251 which is provided on the rear side of the grasping portion 27 and rotatably borne on the grasping portion 27, and a conversion unit 252 which converts rotation of the handle 251 into rotary movement of the puncture needle 71 and rotary movement of the puncture needle 81. In accordance with an exemplary embodiment, the conversion unit 252 can include a bevel gear 255a fixed to a shaft of the handle 251, a bevel gear 255b meshing with the bevel gear 255a, and a pair of rotary belts 255c and 255d connected to a shaft of the bevel gear 255b.

The rotary belts 255c and 255d each have a pair of pulleys and a belt arranged between and around the pulleys, with one of the pulleys fixed to the shaft of the bevel gear 255b. The rotary belts 255c and 255d are rotated in the same direction and at the same speed, as the bevel gear 255b rotates. The rotary belt 255c is provided in contact with the puncture needle 71, while the rotary belt 255d is provided in contact with the puncture needle 81. When the rotary belts 255c and 255d are rotated by an operation of the handle 251, therefore, the puncture needles 71 and 81 are rotationally moved attendantly on the rotation of the rotary belts 255c and 255d, simultaneously, in opposite directions, and by equal rotational angles. The use of the gears in this manner makes the rotational operation unit 25 simple in configuration.

According to this sixteenth embodiment, also, equivalent or similar effects to those of the aforementioned first embodiment can be produced.

Note that while the frame 2 has the grasping portion 27 in this embodiment, the grasping portion 27 may be omitted. In this case, for example, the handle 251 may be disposed on the back side or upper side of the support portion 26.

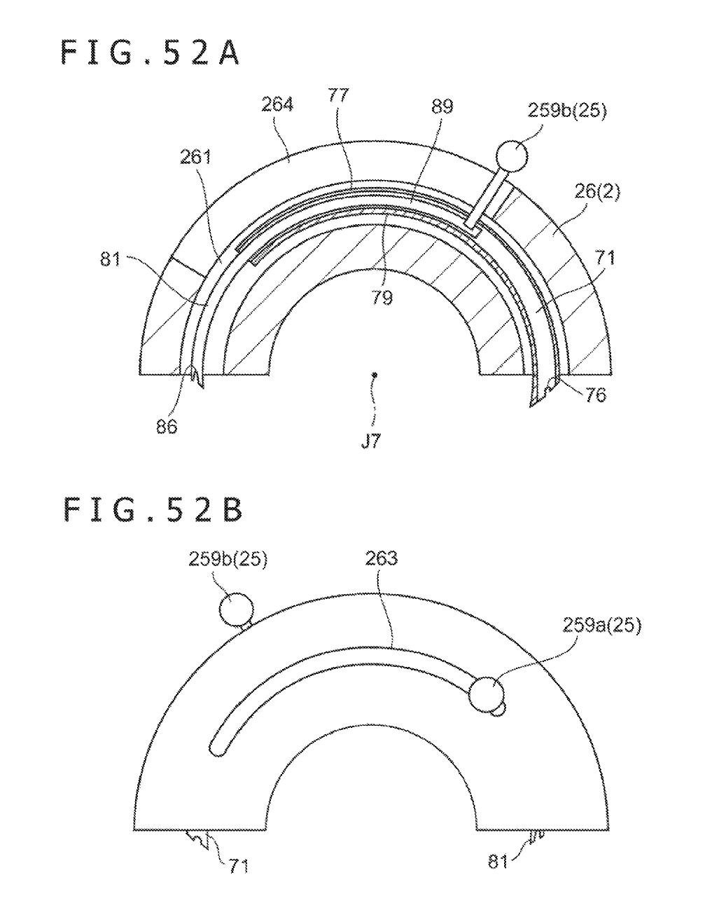

FIGS. 52A and 52B depict views illustrating an initial state of a puncture apparatus according to a seventeenth embodiment of the present disclosure, wherein FIG. 52A is a sectional view as viewed from the front side, and FIG. 52B is a back view.

Referring to these drawings, the seventeenth embodiment of the puncture apparatus will be described below. The following description will focus on differences of the seventeenth embodiment from the aforementioned embodiments, and the description of the same items as above will be omitted.

This seventeenth embodiment is the same as the aforementioned thirteenth embodiment except for a difference in the configuration of puncture needles.

As illustrated in FIGS. 52A and 52B, a puncture apparatus 1 in this embodiment can include a support portion 26, puncture needles 71 and 81, and knobs 259a and 259b. With the knobs 259a and 259b operated manually, the puncture needles 71 and 81 can be operated.

The puncture needles 71 and 81 are supported by and accommodated in the support portion 26, with their distal ends oriented to opposite sides and in the state of both being rotationally movable about a rotational axis J7. The puncture needles 71 and 81 have substantially equal center angles. The puncture needles 71 and 81 are provided in a plane to which the rotational axis J7 is normal, and are rotationally moved in the plane.

The puncture needles 71 and 81 are both tube shaped. A proximal portion 89 of the puncture needle 81 is inserted in a proximal portion 79 of the puncture needle 71. The puncture needle 71 is formed therein with a slit 77 in which to slide a knob 259b connected to the puncture needle 81.

The support portion 26 is substantially U-shaped in form along the shape of the puncture needles 71 and 81. The support portion 26 is provided therein with a guiding hole 261 for guiding the puncture needles 71 and 81 for rotary movement. The support portion 26 is formed in its outer circumferential surface with a slot 264 communicating with the guiding hole 261, and the knob 259b is protruding from the slot 264. The slot 264 is a hole with an end, and abutment of the knob 259b on a distal end portion of the slot 264 can prevent further rotary movement of the puncture needle 81. The support portion 26 is formed in its back surface with a slot 263 communicating with the guiding hole 261, and a knob 259a is protruding from the slot 263. The slot 263 is a hole with an end, and abutment of the knob 259a on a distal end portion of the slot 263 can prevent further rotary movement of the puncture needle 71.

According to this seventeenth embodiment, also, equivalent or similar effects to those of the aforementioned first embodiment can be produced.

While the puncture apparatus and puncturing method according to the present disclosure have been described above with reference to the embodiments illustrated in the attached drawings, the present disclosure is not limited to the details of the above embodiments. The configurations of the components may be replaced by other arbitrary configurations having the same or equivalent functions. In addition, arbitrary structures may be added to the configurations according to the present disclosure. Further, the above embodiments may be used in appropriate combinations thereof.

In the aforementioned first to twelfth embodiments, the puncture needles 71 and 81 are rotationally moved to opposite sides by rotating the handle of the rotational operation unit. However, the rotational operation unit may be omitted, and the puncture needles 71 and 81 may be moved manually. In this case, a handle fixed to the shaft portion 73 of the puncture needle 71 and a handle fixed to the shaft portion 83 of the puncture needle 81 may be provided, and these handles may be operated independently, whereby the puncture needles 71 and 81 can be operated. In addition, the puncture needles 71 and 81 can be operated by grasping the interlock portions 72 and 83. In the case of manually operating the puncture needles 71 and 81 in this manner, puncturing by the puncture needle 71 may be followed by, or may be preceded by, puncturing by the puncture needle 81. Further, puncturing by the puncture needle 71 and puncturing by the puncture needle 81 may be performed simultaneously. Among these puncturing modes, the mode in which puncturing by the puncture needle 71 is followed by puncturing by the puncture needle 81 is preferred. Where puncturing by the puncture needle 71 which is thicker (larger in diametric size) is conducted first, the distal portions of the puncture needles 71 and 81 can be made to engage each other in the living body with enhanced reliability.

In the aforementioned embodiments, description has been made of the case where the puncture apparatus is applied to an apparatus for use in embedding in a living body an implant embeddable for treatment of female urinary incontinence. The use of the puncture apparatus, however, is not limited to the described one.

For example, the target of the application of the present disclosure can include excretory disorders attendant on the weakening of the pelvic floor muscle group (urinary urgency, frequent urination, urinary incontinence, fecal incontinence, urinary retention, strangury or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues include bladder, vagina, uterus, bowel and the like. Less active tissues include bones, muscles, fascias, ligaments and the like. In particular, in the case of pelvic floor disorders, the less active tissues include an obturator fascia, a coccygeus fascia, a cardinal ligament, an uterosacral ligament, a sacrospinous ligament and the like.

For the procedure for interlocking an overactive tissue in the pelvic floor disorder with the less active tissue, there are included retropubic sling surgery, transobturator sling surgery (Transobturator Sling surgery, Transobturator Tape: TOT), tension-free vaginal mesh (Tension-free Vaginal Mesh: TVM) surgery, uterosacral ligament suspension (Uterosacral Ligament Suspension: USLS) surgery, sacrospinous ligament fixation (Sacrospinous Ligament Fixation: SSLF) surgery, iliococcygeus fascia fixation surgery, coccygeus fascia fixation surgery, and the like.

The puncture apparatus disclosed herein can include a first puncture needle, which is curved, the first puncture needle being provided in a rotationally movable manner; and a second puncture needle, which is curved, the second puncture needle being provided in a rotationally movable manner. In the puncture apparatus, when the first puncture needle and the second puncture needle are both rotationally moved toward their distal sides, a distal end portion of the first puncture needle and a distal end portion of the second puncture needle engage each other. Since a through-hole can be formed in a living body by use of the first puncture needle and the second puncture needle, therefore, the first puncture needle and the second puncture needle can be shortened in length, and deformation (flexure, torsion or the like) of the first puncture needle and the second puncture needle at the time of puncturing can be restrained. Accordingly, accurate puncturing by the puncture needles can be performed. Further, the burden on the patient is slight, and safety for the patient is high. In addition, a living body tissue-supporting indwelling article can be easily embedded into a living body.

The puncturing method disclosed herein can include rotationally moving a first puncture needle toward its distal side to cause the first puncture needle to puncture a living body from a first part of the living body, rotationally moving a second puncture needle toward its distal side to cause the second puncture needle to puncture the living body from a second part different from the first part of the living body, and causing a distal portion of the first puncture needle and a distal portion of the second puncture needle to engage each other in the living body. Therefore, the first puncture needle and the second puncture needle can be made shorter in length, and deformation (flexure, torsion or the like) of the first puncture needle and the second puncture needle at the time of puncturing can be restrained, which can help ensure accurate puncturing by use of the puncturing needles. Further, the burden on the patient is lessened, the safety for the patient is enhanced, and a living body tissue-supporting indwelling article can be easily embedded into a living body.

Therefore, the puncture apparatus and puncturing method according to the present disclosure have industrial applicability.

The detailed description above describes a puncture apparatus and a puncturing method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A puncture apparatus comprising:
a first puncture needle which is curved, the first puncture needle provided in a rotationally movable manner; and
a second puncture needle which is curved, the second puncture needle provided in a rotationally movable manner,
wherein when the first puncture needle and the second puncture needle are both rotationally moved toward their distal sides, a distal portion of the first puncture needle and a distal portion of the second puncture needle engage each other.

2. The puncture apparatus according to claim 1, comprising:
a urethra-insertion portion having an elongated shape, the urethra-insertion portion configured to be inserted into a urethra; and
a restriction unit adapted to restrict positional relationships between the first puncture needle and the second puncture needle and the urethra-insertion portion so that when the first puncture needle and the second puncture needle are rotationally moved toward their distal sides, a distal end of at least one of the first puncture needle and the second puncture needle passes on a farther side from a rotational center of the first puncture needle and the second puncture needle than the urethral-insertion portion.

3. The puncture apparatus according to claim 2, comprising:
a vaginal-insertion portion having an elongated shape, the vaginal-insertion portion configured to be inserted into a vagina,
wherein the restriction unit restricts positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion.

4. The puncture apparatus according to claim 2, comprising:
a first shaft portion connected to the first puncture needle, the first shaft portion constituting a rotational shaft for rotary movement of the first puncture needle; and
a second shaft portion connected to the second puncture needle, the second shaft portion constituting a rotational shaft for rotary movement of the second puncture needle,
wherein the restriction unit has a support portion supporting the first shaft portion and the second shaft portion in a rotationally movable manner, the support portion supporting the urethral-insertion portion.

5. The puncture apparatus according to claim 2, comprising:
a vaginal-insertion portion having an elongated shape, the vaginal-insertion portion configured to be inserted into a vagina;
a first shaft portion connected to the first puncture needle, the first shaft portion constituting a rotational shaft for rotary movement of the first puncture needle; and
a second shaft portion connected to the second puncture needle, the second shaft portion constituting a rotational shaft for rotary movement of the second puncture needle,
wherein the restriction unit restricts positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion, and the restriction unit has a support portion supporting the first shaft portion and the second shaft portion in a rotationally movable manner, the support portion supporting the urethral-insertion portion and the vaginal-insertion portion.

6. The puncture apparatus according to claim 2,
wherein a rotational axis of each of the first puncture needle and the second puncture needle is inclined against an axis of the urethral-insertion portion so that a separated distance between the rotational axis and the axis of the urethral-insertion portion increases or decreases along a distal direction.

7. A method of puncturing a living body, the method comprising:
rotationally moving a first puncture needle toward its distal side to cause the first puncture needle to puncture the living body from a first part of the living body;
rotationally moving a second puncture needle toward its distal side to cause the second puncture needle to puncture the living body from a second part different from the first part of the living body; and
engaging a distal portion of the first puncture needle and a distal portion of the second puncture needle to each other in the living body.

8. The method according to claim 7, comprising:
inserting a urethra-insertion portion having an elongated shape into a urethra; and
restricting positional relationships between the first puncture needle and the second puncture needle and the urethra-insertion portion so that when the first puncture needle and the second puncture needle are rotationally moved toward their distal sides, a distal end of at least one of the first puncture needle and the second puncture needle passes on a farther side from a rotational center of the first puncture needle and the second puncture needle than the urethral-insertion portion.

9. The method according to claim 8, comprising:
inserting a vaginal-insertion portion having an elongated shape into a vagina; and
restricting positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion.

10. The method according to claim 8, comprising:
a first shaft portion connected to the first puncture needle, the first shaft portion constituting a rotational shaft for rotary movement of the first puncture needle; and
a second shaft portion connected to the second puncture needle, the second shaft portion constituting a rotational shaft for rotary movement of the second puncture needle.

11. The method according to claim 10, comprising:
supporting the first shaft portion and the second shaft portion in a rotationally movable manner with a support portion, the support portion supporting the urethral-insertion portion.

12. The method according to claim 8, comprising:
a first shaft portion connected to the first puncture needle, the first shaft portion constituting a rotational shaft for rotary movement of the first puncture needle; and
a second shaft portion connected to the second puncture needle, the second shaft portion constituting a rotational shaft for rotary movement of the second puncture needle.

13. The method according to claim 12, comprising:
inserting a vaginal-insertion portion having an elongated shape into a vagina; and
restricting positional relationships between the first puncture needle and the second puncture needle and the vaginal-insertion portion so that when the first puncture needle and the second puncture needle are rotationally moved toward their distal sides, a distal end of the first puncture needle and a distal end of the second puncture needle do not collide against the vaginal-insertion portion, and a support portion supporting the first shaft portion and the second shaft portion in a rotationally movable manner, the support portion supporting the urethral-insertion portion and the vaginal-insertion portion.

14. The method according to claim 8, comprising:
inclining a rotational axis of each of the first puncture needle and the second puncture needle against an axis of the urethral-insertion portion so that a separated distance between the rotational axis and the axis of the urethral-insertion portion increases or decreases along a distal direction.
